(19) United States
(12) Patent Application Publication
(10) Pub. No.: US 2005/0013803 A1
(43) Pub. Date: Jan. 20, 2005

(54) Diagnosing and treating cancer cells using mutant viruses

(76) Inventor: Thomas L. Benjamin, Cambridge, MA (US)

Correspondence Address: CLARK & ELBING LLP, 101 FEDERAL STREET, BOSTON, MA, 02110, US

(21) Appl. No.: 10/828,815

(22) Filed: Apr. 21, 2004

Related U.S. Application Data
(63) Continuation-in-part of application No. 10/765,520, filed on Jan. 27, 2004.
Application No. 10/765,520 is a continuation of application No. 09/812,471, filed on Mar. 19, 2001.
Application No. 10/765,520 is a continuation-in-part of application No. 09/988,117, filed on Nov. 16, 2001, now abandoned.
Application No. 09/988,117 is a continuation-in-part of application No. 09/812,633, filed on Mar. 19, 2001, now abandoned.
Application No. 10/765,520 is a continuation-in-part of application No. 10/316,532, filed on Dec. 10, 2002.
(60) Provisional application No. 60/216,723, filed on Jul. 7, 2000.
(60) Provisional application No. 60/216,723, filed on Jul. 7, 2000.
(60) Provisional application No. 60/339,140, filed on Dec. 10, 2001.

(51) Int. Cl. A61K 48/00; C12N 15/86

(52) U.S. Cl. 424/93.2; 435/456

(57) ABSTRACT

The invention provides methods for the identification of genes and their encoded proteins involved in the susceptibility to proliferative disorders, including cancer, using a tumor host range mutant virus (T-HR mutant). In addition, the invention provides methods for the diagnosis of abnormally proliferating cells in a subject, using a T-HR mutant. The invention also features, T-HR mutants that can be used to kill cancer cells such as ones carrying a Sal2 alteration. Furthermore, the invention features the analysis of Sal2 nucleic acids and proteins for diagnosing and treating patients having proliferative disorders, including cancer, involving mutations in a Sal2 gene and encoded protein. Also encompassed by the invention are transgenic and knockout mice including Sal2 nucleic acids or proteins and mutants thereof.

### PRIORITY INFORMATION

This application is a Continuation-In-Part of U.S. application Ser. No. 10/765,520, filed Jan. 27, 2004, which is a Continuation of U.S. application Ser. No. 09/812,471, filed Mar. 19, 2001 and a Continuation-In-Part of U.S. application Ser. No. 09/988,117, filed Nov. 16, 2001, which in turn is a Continuation-In-Part of U.S. application Ser. No. 09/812,633, filed Mar. 19, 2001. All of these applications claim the benefit of U.S. Provisional Application No. 60/216,723, filed Jul. 7, 2000. U.S. application Ser. No. 10/765,520 is further a Continuation-In-Part of U.S. application Ser. No. 10/316,532, filed Dec. 10, 2002, which claims the benefit of U.S. Provisional Application No. 60/339,140, filed Dec. 10, 2001. These disclosures are hereby incorporated by reference.

### STATEMENT AS TO FEDERALLY SPONSORED RESEARCH

The present research was supported by a grant from the National Cancer Institute (Number R35 CA44343). The U.S. government has certain rights to this invention.

### FIELD OF THE INVENTION

The field of the invention is regulation of cellular proliferation.

### BACKGROUND OF THE INVENTION

Transforming genes of DNA tumor viruses perform essential functions in virus growth, acting largely as proto-oncogene activators or tumor suppressor gene inactivators. The isolation and characterization of mutant viruses that are able to propagate in cells containing a mutation in known proto-oncogene or tumor suppressor genes has been useful in identifying and studying the viral equivalents or interactors of these genes. The transforming gene of the highly oncogenic murine polyoma virus was identified through studies of host range mutants isolated using polyoma transformed 3T3 cells as the permissive host and normal 3T3 cells as the non-permissive host. This approach requires expression a known viral protein by the permissive host, since it is based on the idea of complementation between cell-associated wild-type viral genes and an infecting virus mutant. In addition to its use with polyoma virus, the complementation approach has also been successfully used with other oncogenic DNA viruses, e.g., with 293 cells expressing adenovirus E1A/E1B genes and COS cells expressing the SV40 large T antigen. Complementing cell lines have also been used in other systems to propagate specifically defective virus mutants for vaccine development and other purposes. However, by design, these types of systems rely on permissive hosts constructed with known gain-of-function mutations and are only applicable to mutants in known viral genes, as well as to viruses with known mutations, since the host cell must express a functional version of the mutant viral protein.

The use of mutant adenoviruses unable to inactivate p53 or the retinoblastoma protein (pRb) to kill cancer cells lacking one of these proteins has been previously described (patent Nos. U.S. Pat. No. 5,677,178 and WO 94/18992). It was well known prior to these observations that these two genes are mutated in a variety of cancers.

While a number of genes are known to be involved in the progression towards cancer, there is a significant need for the development of a general, unbiased method for identifying new genes involved in the pre-disposition for, or progression of cancer or other proliferative disorders. Furthermore, methods for diagnosing and treating patients with mutations in known as well as newly identified genes would greatly aid in the management of cancer.

### SUMMARY OF THE INVENTION

The invention features novel tumor host range viruses, for identifying mammalian cancer susceptibility genes, such as tumor suppressor genes and proto-oncogenes, and methods for diagnosing and treating patients having proliferative disorders, such as cancers, involving mutations in such genes.

The tumor host range mutant viruses (T-HR mutants) used in the methods of the invention contain mutations that prevent the virus from propagating in normal cells. These viruses are, however, able to propagate in abnormally proliferating cells because of genetic changes that are present in these cells, such as the inactivation of tumor suppressor genes or the activation of proto-oncogenes. A T-HR mutant that infects a normal cell is unable to propagate in such a cell because it is unable to inactivate a tumor suppressor gene or to activate a proto-oncogene due to a mutation in the viral genome. In contrast, if this T-HR mutant infects an abnormally proliferating cell that already has a tumor suppressor gene inactivated, this virus is able to propagate. Likewise, if such a T-HR mutant infects an abnormally proliferating cell that contains an activated proto-oncogene, the virus is also able to propagate.

An exemplary tumor suppressor gene identified according to the methods of the invention is Sal2. The invention therefore features the use of Sal2 nucleic acids and proteins in methods of identifying a mammal having, or at risk of acquiring, a proliferative disease such as a cancer involving mutations in a Sal2 gene and encoded protein.

The invention features novel T-HR mutant viruses, new primary cellular targets for DNA tumor viruses, such as Taz, as well as cellular factors, such as Death Inducer with SAP Domain (DIS) polypeptides, that interact with these primary targets. In addition, the invention encompasses DIS nucleic acid and amino acid sequences. The compounds described herein may be used in methods for diagnosing and treating patients having proliferative disorders, such as cancers.

The Tumor Host Range Mutant System

According to the present invention, T-HR mutant viruses are identified using the method outlined in table 1.

TABLE 1
Tumor Host Range Mutants - Selection Procedure
and Target Identification

| | |
|---|---|
| I. Mutant | 1. Random mutagenesis of wild-type viral DNA |
| Selection | 2. Amplification of the mutant virus |
| | by growth in tumor cells |
| | 3. Cloning by plaque isolation on tumor cells |
| | 4. Screening of plaque lysates for |
| | the absence of growth in normal cells |
| | 5. Molecular cloning and sequencing of |
| | the mutant viral DNA |
| II. Target | 6. Screening of a mouse embryo cDNA library |
| Identification | in yeast with wild-type bait |
| and | 7. Counterscreening positive clones for lack |
| Validation | of interaction with mutant bait |
| | 8. Construction of complete cDNA expressing |
| | the target protein |
| | 9. Verification of viral protein-cellular target |
| | interactions in vitro and in vivo (e.g., |
| | T antigen-cellular protein |
| | interactions). |
| III. Identification | 10. Sequencing DNA derived from |
| of Risk | a tumor |
| Factors | 11. Sequencing DNA derived from normal |
| | tissue of the same patient |
| | 12. Using the sequence information to |
| | establish whether the |
| | mutation is somatic or germline |
| | 13. Using this information in an |
| | epidemiological study to |
| | assess risk factors in a population |

Accordingly, T-HR mutants may be identified using the following steps: (a) providing a viral DNA (e.g., wild-type); (b) introducing random mutations in the viral DNA, thereby obtaining a collection of mutant viruses; (c) infecting abnormally proliferating cells with the obtained collection of mutant viruses to amplify mutant viruses; (d) selecting mutant viruses from the collection that have the ability to proliferate in abnormally proliferating cells by plaque isolation; (e) infecting normally proliferating cells with the selected mutant viruses; (f) identifying mutant viruses from step (e) that do not proliferate in the normally proliferating cells, thereby identifying THR mutant viruses. Optionally, the collection of mutant viruses obtained in step (b) is uncharacterized.

Since a T-HR mutant is unable to propagate in normal cells, but is able to propagate in abnormally proliferating cells, one aspect of the invention features a method of using T-HR mutants to identify a cellular protein that is involved in the susceptibility to cancer and other proliferative disorders. This method involves: (a) infecting a normal cell and an abnormally proliferating cell with a collection of uncharacterized mutant viruses; (b) identifying a mutant virus from the collection that can grow in an abnormally proliferating cell and can not grow in a normal cell (i.e., a T-HR mutant); (c) identifying the mutated viral gene or mutated protein in the virus, where this mutation allows the virus to grow on the abnormally proliferating cell; and (d) screening to identify the cellular proteins which interact with the wild-type viral protein, but not with the mutated protein.

In a preferred embodiment of the above aspect of the invention, the abnormally proliferating cell infected with the collection of uncharacterized mutant viruses is also uncharacterized. In an additional preferred embodiment, the cellular and viral proteins can be identified by, for example, using an assay that detects protein-protein interactions (e.g., a GST-pull-down assay). These proteins may be, for example, tumor suppressor proteins or proto-oncogene products; however the retinoblastoma tumor suppressor protein and the gene encoding this protein are specifically excluded from this and all other aspects of the invention. In another preferred embodiment, the method of this aspect is used to isolate a mutant virus (i.e., a T-HR mutant).

Preferred viruses with a mammalian, preferably human, host range used in this and other aspects of the invention include, for example, simian virus 40, human polyoma virus, parnovirus, papilloma virus, herpes virus, and primate adenoviruses.

A further aspect of the invention features a method of determining the presence or absence of an alteration in the genetic material of a cell that involves determining whether such a cell can act as a permissive host for the growth of a characterized T-HR mutant (e.g., (e.g., BMD-13 T-HR mutant virus), where the T-HR mutant is capable of propagating in an abnormally proliferating cell and not capable of propagating in a normal cell. The retinoblastoma and p53 genes are specifically excluded from this aspect of the invention. In a desirable embodiment of this method, the presence of the alteration in the genetic material is indicative of an organism carrying this genetic alteration having, or being at an increased risk of developing, a proliferative disease.

In other embodiments of this aspect, the cell is determined to have an alteration in a Sal2, Taz, a GAP SH3 binding protein, a nucleolin, a Vesicle Associated Protein 1, or a Death Inducer with SAP Domain nucleic acid sequence or polypeptide.

In addition, the BMD-13 T-HR mutant virus may contain an alteration (e.g., an Aspartic Acid to Asparagine substitution) at the second position of the amino acid sequence of any of the polyoma T antigens, or the BMD-13 T-HR mutant virus may contain a deletion of amino acids 2 to 4 of any of the polyoma T antigens.

In a preferred embodiment of the above aspect of the invention, the alteration of the genetic material to be tested for in the cell indicates that the organism carrying this alteration is at an increased risk of developing a proliferative disease. Preferably, this genetic alteration is in a tumor suppressor gene or in a proto-oncogene. In another preferred embodiment, the T-HR mutant has been characterized as being complemented by a mutation in a specific tumor suppressor or proto-oncogene. In an additional preferred embodiment of the above aspects of the invention, the cells used in the methods of the invention are from a mammal, for example, a human.

A further aspect of the invention features a method of identifying a mammal having, or at increased risk of acquiring, a proliferative disease. This method includes determining whether there is a proliferative disease-associated alteration in a Sal2 nucleic acid or protein of the mammal. An example of a proliferative disease-associated Sal2 alteration is the substitution of a Cys for the Ser at position 73 of SEQ ID NO:1. In one embodiment, the method is used to identify a mammal, preferably a human, having a proliferative disease, while in another embodiment, the method is used to identify a mammal at increased risk of acquiring a proliferative disease. In preferred embodiments of this method an antibody specific for either the human, or for a proliferative disease-associated mutant Sal2 protein is used.

In another embodiment of this aspect of the invention, determining whether the mammal has or is at increased risk of acquiring a proliferative disease is done by, for example, polymerase chain reaction (PCR) amplification, single nucleotide polymorphism (SNP) determination, restriction fragment length polymorphism (RFLP) analysis, hybridization analysis, or mismatch detection analysis.

In addition, identifying the alteration may also involve: (i) contacting a first nucleic acid probe which is specific for binding to the human Sal2 nucleic acid containing the alteration with a nucleic acid from a cell from the mammal under conditions which allow the first nucleic acid probe to anneal to complementary sequences in the cell; and (ii) detecting duplex formation between the first nucleic acid probe and the complementary sequences. The nucleic acid probe of step (i), which is, for example, at least 12 contiguous nucleotides in length, may be derived from the human Sal2 nucleic acid containing a proliferative disease-associated alteration. The cell may be from a physiological sample, which may contain, for example, mRNA or the nucleic acid probe of step (i) annealed to the mRNA. Furthermore, another embodiment of this aspect includes a second nucleic acid probe, where the first and second nucleic acid probes are PCR primers, and where the human Sal2 nucleic acid or a fragment is amplified using PCR between steps (i) and (ii).

In another embodiment of this aspect, the cell may be selected from a physiological sample, for example, containing abnormally proliferating or normal tissue, and may be from human tissue, blood, ovarian tissue, bladder tissue, colon tissue, and cells grown in culture.

The present invention also features a method of decreasing proliferation of an abnormally proliferating cell, e.g., an ovarian cell. This method includes the step of contacting the abnormally proliferating cell with a Sal2 nucleic acid sequence, where this contacting results in the expression of a Sal2 polypeptide having tumor suppressive activity in the abnormally proliferating cell. In one embodiment, the Sal2 polypeptide includes the amino acid sequence of SEQ ID NO:1 or 3. In another preferred embodiment, the abnormally proliferating cell has a proliferative disease-associated alteration in a Sal2 nucleic acid sequence, for example, one that results in a polypeptide that contains a substitution of a Cys for the Ser at position 73 of SEQ ID NO:1. The invention also provides an isolated Sal2 nucleic acid sequence, for example, a human Sal2 nucleic acid sequence, encoding a polypeptide that contains a substitution of a Cys for the Ser at position 73 of SEQ ID NO:1.

The invention also features a method of decreasing DNA tumor virus replication and dissemination. This method includes the step of contacting a cell infected with a DNA tumor virus, for example, a simian virus 40, human polyoma virus, herpes virus, primate adenoviruses, parvovirus, or a papilloma virus, with a Sal2 nucleic acid sequence, where this contacting results in the expression of a Sal2 polypeptide in the cell infected with the DNA tumor virus and prevents the DNA tumor virus from replicating and disseminating. In a preferred embodiment of this aspect, the Sal2 polypeptide includes the amino acid sequence provided in SEQ ID NO:1 or 3.

In another aspect, the invention features a method of killing a cell with a proliferative disease that involves: (i) contacting a cell with a proliferative disease, for example, a mammalian cell, with a T-HR mutant; and (ii) allowing the T-HR mutant to lyse this cell. In a preferred embodiment, the TH-R mutant may be a T-HR mutant specific for a cell carrying a Sal2 mutation, for example, the TMD-25 T-HR mutant virus. In an additional preferred embodiment of this aspect, the mammalian cell is from a human. The mammalian cell may also be in a mammal, for example a human, with a proliferative disorder. In a further embodiment, the T-HR mutant may be administered, for example, in a pharmaceutically acceptable carrier. In addition, the T-HR mutant may be administered, for example, by parenteral, intravenous, intraperitoneal, intramuscular, subcutaneous, or subdermal injection. The T-HR mutant, however, may also be administered orally, nasally, topically, or as an aerosol.

Thus, the invention features a method of killing an abnormally proliferating cell, for example, a mammalian cell, such as a human cell. This method involves contacting an abnormally proliferating cell with a T-HR mutant specific for a cell carrying a Taz, a GAP SH3 binding protein, a nucleolin, a Vesicle Associated Protein 1, or a Death Inducer with SAP Domain alteration, and allowing this T-HR mutant to lyse said cell.

In desirable embodiments of this aspect of the invention the T-HR mutant virus is a BMD-13 T-HR mutant virus, for example, one that contains an alteration at the second position of the amino acid sequence of any of the polyoma T antigens. Such an alteration may be, for example, an Aspartic Acid to Asparagine substitution or the deletion of amino acids 2 to 4 of any of the polyoma T antigens. In addition, the T-HR mutant may be administered in a pharmaceutically acceptable carrier.

In another desirable embodiment the T-HR mutant virus is a mutant of a simian virus 40, human polyoma virus, herpes virus, primate adenoviruses, parvovirus, or a papilloma virus.

In a further aspect, the invention features a BMD-13 T-HR mutant virus, for example, one that contains an alteration at the second position of the amino acid sequence of any of the polyoma T antigens. This alteration may be an Aspartic Acid to Asparagine substitution or a deletion of amino acids 2 to 4 of any of the polyoma T antigens.

In another aspect, the invention features an isolated nucleic acid encoding a Death Inducer with SAP Domain amino acid sequence, where this Death Inducer with SAP Domain amino acid sequence is at least 30% identical to the amino acid sequence of SEQ ID NO:2 or SEQ ID NO:4 and induces DNA condensation and apoptosis in a mammalian cell. However, this Death Inducer with SAP Domain amino acid sequence may also include the amino acid sequence of SEQ ID NO:2 or SEQ ID NO:4. In addition, the nucleic acid encoding the Death Inducer with SAP Domain amino acid may include the nucleic acid sequence of SEQ ID NO:1 or SEQ ID NO:3.

In yet another aspect, the invention features a method of killing an abnormally proliferating cell. This method involves contacting the abnormally proliferating cell with a Death Inducer with SAP Domain nucleic acid sequence, where this contacting results in the expression of a DIS polypeptide in the abnormally proliferating cell. The Death Inducer with SAP Domain nucleic acid sequence may include, for example, the nucleic acid sequence of SEQ ID NO:1 or SEQ ID NO:3. In addition, the abnormally proliferating cell may be an endometrial, prostate, or ovarian cell.

In a further aspect, the invention features a method of identifying a mammal, for example, a human, having or at increased risk of acquiring a proliferative disease. This method includes the step of determining whether there is a loss of heterozygosity in a Death Inducer with SAP Domain nucleic acid of the mammal, where a loss of heterozygosity in a Death Inducer with SAP Domain nucleic acid is indicative of the mammal having, or being at risk of acquiring a proliferative disease. In a desirable embodiment, this method is used to identify a mammal having a proliferative disease and in another desirable embodiment, this method is used to identify a mammal at increased risk of acquiring a proliferative disease. In further desirable embodiments of this aspect, the method involves the use of polymerase chain reaction (PCR) amplification, single nucleotide polymorphism (SNP) determination, restriction fragment length polymorphism (RFLP) analysis, hybridization analysis, or mismatch detection analysis.

An additional aspect of the invention features a method of decreasing proliferation of an abnormally proliferating cell. This method includes the step of contacting the abnormally proliferating cell with a Taz nucleic acid sequence, where this contacting results in the expression of a Taz polypeptide having wild-type activity in the abnormally proliferating cell.

In yet another aspect, the invention features a method of decreasing virus, for example, tumor virus, replication and dissemination. This method includes the step of contacting a cell infected with a virus with a T-HR mutant target gene nucleic acid sequence, where this contacting results in the expression of a T-HR mutant target gene encoded polypeptide in the cell infected with the virus and prevents the virus from replicating and disseminating, or, for instance, from replicating or disseminating. For example, the virus may be a DNA tumor virus. In addition, in desirable embodiments, the T-HR mutant target gene nucleic acid sequence may be a Taz, a GAP SH3 binding protein, a nucleolin, a Vesicle Associated Protein 1, or a Death Inducer with SAP Domain nucleic acid sequence, such as the Death Inducer with SAP Domain nucleic acid sequence of SEQ ID NO:1 or SEQ ID NO:3.

In a further aspect, the invention features a method of identifying a compound which modulates cell proliferation. This method involves: a) exposing a cell or a cell extract to a test compound, and b) measuring whether the test compound modulates Taz, Nucleolin, Vesicle Associated Protein 1, or Death Inducer with SAP Domain levels, relative to Taz, Nucleolin, Vesicle Associated Protein 1, or Death Inducer with SAP Domain levels in a cell or cell extract not exposed to the test compound. In desirable embodiments of this aspect of the invention Taz, Nucleolin, Vesicle Associated Protein 1, or Death Inducer with SAP Domain is a Taz, Nucleolin, Vesicle Associated Protein 1, or Death Inducer with SAP Domain polypeptide or a Taz, Nucleolin, Vesicle Associated Protein 1, or Death Inducer with SAP Domain nucleic acid. In additional desirable embodiments, Taz, Nucleolin, Vesicle Associated Protein 1, or Death Inducer with SAP Domain polypeptide or nucleic acid levels may be measured. In further desirable embodiments, the compound identified using the method of the twelfth aspect of the invention may be used to treat a proliferative disease, for example, one that is due to a proliferative disease-associated alteration in a Taz, Nucleolin, Vesicle Associated Protein 1, or Death Inducer with SAP Domain nucleic acid sequence. Desirable examples of proliferative diseases include cancers such as leukemias or ovarian cancer.

An additional aspect of the invention features a knockout mouse including a knockout mutation in a genomic Death Inducer with SAP Domain nucleic acid sequence and the tenth aspect features a transgenic mouse whose genome includes a nucleic acid construct containing a Death Inducer with SAP Domain nucleic acid sequence which is operably linked to transcriptional regulatory elements, for example, a tissue specific promoter, and encodes a Death Inducer with SAP Domain polypeptide. Optionally, the Death Inducer with SAP Domain polypeptide is mutant. Another feature of the invention is a cell line derived from cells isolated from the transgenic mouse described above.

A further aspect of the invention encompasses a knockout mouse featuring a knockout mutation in a genomic mSal2 gene. This knockout mouse may also contain, for example, a nucleic acid construct including a mutant Sal2 gene and this mutant Sal2 gene may be conditionally expressed. In a preferred embodiment, the mutant Sal2 gene, for example a human Sal2 gene, encodes a protein that contains a substitution of a Cys for the Ser at position 73 of SEQ ID NO:1. However, the Sal2 protein may also be wild-type.

An additional aspect of the invention features a transgenic mouse whose genome includes a nucleic acid construct that contains a Sal2 nucleic acid, which is operably linked to transcriptional regulatory elements and encodes a Sal2 protein, e.g., a mutant Sal2 protein. The mutant Sal2 protein may also be a human Sal2 protein, for example, one that has a modification of function. Furthermore, the transgenic mouse may contain a mouse Sal2 protein, e.g., the protein of SEQ ID NO:3. This mouse Sal2 protein may be mutant, such as a mouse Sal2 protein containing the substitution of a Cys for the Ser at position 73 of SEQ ID NO:3.

In preferred embodiments of this aspect, the transcriptional regulatory elements include a promoter that is a tissue-specific promoter, such that the nucleic acid is expressed, and the protein is produced at detectable levels, in cells selected from the group consisting of ovarian, bladder, and colon cells. However, the transcriptional regulatory element may also include the wild-type Sal2 promoter.

In a further embodiment of this aspect, the transgenic mouse develops ovarian tumors, and these tumors may metastasize. The invention also includes a cell line, such as an ovarian cell line, derived from cells isolated from the transgenic mouse.

An additional aspect, the invention encompasses a method of identifying a compound which alters cell proliferation, the method involving: a) contacting a first cell with a test compound, and b) measuring whether the test compound alters proliferation in the first cell, relative to a second cell not contacted with the test compound, wherein the first and second cells have a proliferative disease-associated alteration in a Sal2 nucleic acid. In a preferred embodiment of this aspect, the ability of the test compound to alter proliferation is determined by measuring the ability of a virus, for example, a T-HR mutant virus, to propagate in the first cell contacted with the test compound, relative to the second cell not contacted with the test compound. In addition, the first and second cells may be mammalian cells, for example, human cells. Furthermore, these cells may be ovarian cells. The cells may also be in the same mammal or in different mammals and the mammal may be a transgenic mouse or a knockout mouse containing a knockout mutation in a genomic mSal2 gene.

A final aspect of the invention features a method of identifying a compound which alters cell proliferation, the method involving: a) exposing a cell or a cell extract to a test compound, and b) measuring whether the test compound alters Sal2 levels, for example, Sal2 protein or nucleic acid levels, relative to Sal2 levels in a cell or cell extract not exposed to the test compound. In a preferred embodiment of this aspect, the cell has a proliferative disease-associated alteration in a Sal2 nucleic acid or the extract is from a cell having a proliferative disease-associated alteration in a Sal2 nucleic acid. This cell or cell extract may be mammalian, e.g., human. Furthermore, the cell may be in a mammal, for example, a transgenic mouse whose genome includes a nucleic acid construct containing a Sal2 nucleic acid, which is operably linked to transcriptional regulatory elements and encodes a Sal2 protein, or a knockout mouse comprising a knockout mutation in a genomic mSal2 gene. In another embodiment, the exposing in step a) of this aspect is done with a cell and this cell is an ovarian cell.

Definitions

By “DNA tumor virus,” as used herein is meant a virus that has a mammalian (e.g., rodent, primate, or human) host range that can, as a consequence of infecting the cell, transform a normal cell into an abnormally proliferating cell. Examples of “DNA tumor viruses” include simian virus 40, human polyoma virus, parvovirus, papilloma virus, herpes virus, and primate adenovirus.

“Tumor host range mutant virus (T-HR mutant),” as used herein, refers to a virus that has a reduced ability to replicate and disseminate in a normal cell, relative to the replication of a wild-type virus in the same type of cell, but is able to replicate and disseminate in a cell having abnormal proliferation. The abnormally proliferating cell may, for example, have one or more mutations in a gene or genes involved in the regulation of cell growth, of the cell cycle, or of programmed cell death (e.g., apoptosis). These genes include, for example, tumor suppressor genes and proto-oncogenes, but any cellular gene that a virus must inactive or activate in order to grow is also included. Adenoviruses having mutations in the p53 and retinoblastoma genes are specifically excluded.

Reference herein to a “collection of uncharacterized mutant viruses” refers to a sample of viruses where at least one of the viruses, in a collection of at least 1000 viruses, (e.g., 0.1%) carries at least one mutation in at least one of the genes of the viral genome. Preferably, at least 10%, 25%, 30%, or 50% of the viruses in this collection carry at least one mutation in at least one of the genes in the viral genome. In addition, such mutations preferably inactivate viral proteins that are necessary for transforming a host cell into a cancer cell. The types of mutations that may be present in the viral genes include, for example, point mutations, deletions, insertions, duplications, and inversions. Furthermore, the mutations may result in modification of function, such as a partial or a complete loss-of-function of the viral gene. Preferably the virus has a mammalian host range (e.g., rodent or primate), most preferably a human host range. Viruses that may be used in such a collection include, for example, simian virus 40, human polyoma virus, parnovirus, papilloma virus, herpes virus, and primate adenoviruses. However, any virus that needs to overcome a cell cycle checkpoint or affect a signal transduction pathway in order to propagate may be used in this collection.

“T-HR mutant specific for a Sal2 mutation,” as used herein, refers to a TH-R mutant virus that is able to propagate in a cell containing a genetic alteration in a Sal2 gene. For example, the “T-HR mutant specific for a Sal2 mutation” may be the TMD-25 T-HR mutant virus described herein.

“Sal2 mutation”, as used herein, refers to a genetic change in the nucleic acid sequence of a Sal2 gene, for example, SEQ ID NO:2 and SEQ ID NO:4, which results in the abnormal proliferation, or predisposition to abnormal proliferation, of a cell carrying such a change. Preferably, the genetic change is a missense mutation. Most preferably, the mutation is a substitution of a Cys for the Ser at position 73 of SEQ ID NO:1.

By a “Sal2 nucleic acid sequence” as used herein is meant a nucleic acid sequence that is at least 40%, 50%, 60%, 70%, 75%, 80%, 85%, 90%, 95%, or 99% identical to a nucleic acid sequence provided in SEQ ID NO:2 or 4 over a region comprising at least 200, 300, 500, 750, 1000, 1500, 2000, 2500, 3000, or 3500 contiguous nucleotides. In addition a “Sal2 nucleic acid sequence” may be identical to a nucleic acid sequence provided in SEQ ID NO:2 or 4. Desirably, a “Sal2 nucleic acid sequence” is a human or a mouse Sal2 nucleic acid sequence.

By a “Sal2 protein” or a “Sal2 polypeptide,” as used herein is meant an amino acid sequence that is at least 30%, 40%, 50%, 60%, 70%, 75%, 80%, 85%, 90%, 95%, or 99% identical to the amino acid sequence of SEQ ID NO:1 or 3 over a region comprising at least 50, 75, 100, 200, 300, 500, 700, 900, or 1000 contiguous amino acids. In addition a “Sal2 polypeptide” or “Sal2 protein” may be identical to the amino acid sequence provided in SEQ ID NO:1 or 3. Desirably, a “Sal2 polypeptide” or “Sal2 protein” is a human or a mouse Sal2 polypeptide or amino acid sequence.

By a “Sal2 polypeptide having tumor suppressive activity” is meant a polypeptide that reduces the ability of a TMD-25 T-HR mutant to replicate and disseminate in a cell relative to a Sal2 polypeptide having a substitution of a Cys for the Ser at position 73 of SEQ ID NO:1. In one embodiment, the reduction in the ability of a TMD-25 T-HR to replicate and disseminate is at least 25%. In more desirable embodiments, the reduction in the ability of a TMD-25 T-HR mutant to replicate and disseminate is at least 50%, 75%, 80%, 90%, or 95%. However, a “Sal2 polypeptide having tumor suppressive activity” may also completely block the ability of a TMD-25 T-HR mutant to replicate and disseminate. The reduction in the ability of a TMD-25 T-HR mutant to replicate and disseminate may be measured by determining the number of cells infected with the TMD-25 T-HR mutant that survive in presence of a “Sal2 polypeptide having tumor suppressive activity” in comparison to the number of cells infected with the TMD-25 T-HR mutant that survive in the absence of a “Sal2 polypeptide having tumor suppressive activity,” where an increase in the number of cells surviving is indicative of a reduction in ability of the TMD-25 T-HR mutant to replicate and disseminate.

“Uncharacterized abnormally proliferating cell,” as used herein, refers to a cell where the cause of the abnormal proliferation is unknown. For example, the genetic alteration that results in abnormal proliferation has not been identified. However, other features of the cell may be characterized.

“Cancer susceptibility gene,” as used herein, refers to any gene that, when altered, increases the likelihood that the organism carrying the gene will develop a proliferative disorder during its lifetime. Examples of such genes include proto-oncogenes, tumor suppressor genes, and genes involved in the regulation of cell growth, the cell cycle, and apoptosis.

“Proliferative disease,” as used herein, refers to any genetic change within a differentiated cell that results in the abnormal proliferation of a cell. Such changes include mutations in genes involved in the regulation of the cell cycle, of growth control, or of apoptosis and can further include tumor suppressor genes and proto-oncogenes. Specific examples of proliferative diseases are the various types of cancer, such as ovarian cancer. However, proliferative diseases may also be the result of the cell becoming infected with a transforming virus.

“Proliferative disease-associated alteration,” as used herein, refers to any genetic change within a differentiated cell that results in the abnormal proliferation of a cell. Preferably, such a genetic change correlates with a statistically significant (e.g., the p-value is less than or equal to 0.05) increase in the risk of acquiring a proliferative disease. Examples of such genetic changes include mutations in genes involved in the regulation of the cell cycle, of growth control, or of apoptosis and can further include mutations in tumor suppressor genes and proto-oncogenes. A further example of a proliferative disease-associated alteration is the substitution of a Cys for the Ser at position 73 of SEQ ID NO:1.

“Abnormal proliferation,” as used herein, refers to a cell undergoing cell division that normally does not undergo cell division or that undergoes cell division at an increased frequency when compared to a wild-type cell.

The term “alteration,” when used herein, in reference to a gene, refers to a change in the nucleic acid sequence. Such a change may include, for example, insertions, deletions, and substitutions of one or more nucleic acids, as well as inversions and duplications.

“Genetic lesion,” as used herein, refers to a nucleic acid change. Examples of such a change include single nucleic acid changes as well as deletions and insertions of one or more nucleic acid. However, genetic lesions can also include duplications and inversions. In addition, a genetic lesion may be a naturally-occurring polymorphism, for example, one that predisposes an organism carrying the polymorphism to acquiring a proliferative disease.

“Polymorphism,” as used herein, refers to an alteration in a nucleic acid sequence, for example, a gene. Such an alteration may result in a codon change, which in turn may result in, for example, the substitution of a Cys for the Ser at position 73 of SEQ ID NO:1.

“Modification of function,” as used herein, refers to a change in the function of the protein. Such a change can, for example, result in the partial or complete loss of function, but it can also result in a gain of function.

As used herein, the term “promoter” is intended to encompass transcriptional regulatory elements, that is, all of the elements that promote or regulate transcription, including core elements required for basic interactions between RNA polymerase, transcription factors, upstream elements, enhancers, and response elements.

“Operably linked,” as referred to herein, describes the functional relationship between nucleic acid sequences, for example, a promoter sequence, and a gene to be expressed. Operably linked nucleic acids may be part of a contiguous sequence. However a physical link is not necessary for two nucleic acid sequences to be operably linked. For example, enhancers can exert their effect over long distances and therefore do not require a physical link in sequence to the gene whose transcription they affect.

Reference herein to the “transcriptional regulatory elements” of a gene or a class of genes includes both the entire gene as well as an intact region of naturally-occurring transcriptional regulatory elements. Also included are transcription regulatory elements modified by, for example, rearrangement of the elements, deletion of some elements or of extraneous sequences, and insertion of heterologous elements.

“Conditionally expressed,” as used herein, refers to any method that may be used to control expression of a gene, such as a transgene. These methods may, for example, include the use of promoters that are regulated by a substance, such as tetracycline, that can be administered to the organism, or of promoters that are only active at certain stages of development or in certain tissues. In addition, conditional expression may involve inactivating a gene, for example, by FLP/FRT- or Cre-lox-mediated recombination.

The term “restriction fragment length polymorphism (RFLP) analysis,” as used herein, refers to a method of determining whether an organism carries a specific nucleic acid sequence, for example, a specific alteration in a gene. This method may involve, for example, amplification of a nucleic acid from the organism, followed by cleavage of the nucleic acid with an enzyme, such as a restriction enzyme, and visualizing the products of the cleavage reaction. Furthermore, the cleavage products may be compared to control reactions.

As used herein, “alters proliferation” refers to any change in the proliferation of a cell. For example, this term can be used to describe an increase or a decrease in the rate of cell division. In addition, an alteration of proliferation may refer to a normally quiescent cell entering into the cell cycle or a normally dividing cell ceasing to enter into the cell cycle.

“Measuring protein levels,” as used herein, includes any standard assay used in the art to either directly or indirectly determine protein levels. Such assays, for example may include the use of an antibody, Western analysis, Bradford assays, and spectrophotometric assays.

“Measuring nucleic acid levels,” as used herein, includes any standard assay used in the art to either directly or indirectly determine nucleic acid levels. Such assays include, for example, hybridization analysis, gel electrophoresis, Northern blots, Southern blots, and spectrophotometric assays.

“Decreasing proliferation,” as used herein, refers to a reduction in proliferation of at least 25%, 50%, 70%, 80%, or 90% when compared with control cells. In a desirable embodiment the reduction in proliferation is at least 95%. In a more desirable embodiment the reduction in proliferation is 100%.

By “reduced ability to replicate and disseminate,” as used herein is meant a reduction in the ability of a virus to replicate and disseminate, relative to a wild-type virus in the same type of cell, of at least 50%, 60%, 70%, 80%, 90%, 95%, 99% or the complete inability to replicate or disseminate. Desirably, the ability to replicate and disseminate is reduced by at least 90%, 95%, or 99%.

By “decreasing replication and dissemination,” as used herein is meant a reduction in virus replication and dissemination in the presence of a compound, for example, a Sal2 nucleic acid having tumor suppressive activity, that is at least 50%, 60%, 70%, 80%, 90%, 95%, 99% less than the amount of virus replication and dissemination seen under control conditions in the absence of the compound. Desirably, replication and dissemination is reduced by at least 90%, 95%, or 99%.

By “tumor virus,” as used herein is meant a virus that has a mammalian (e.g., rodent, primate, or human) host range that can, as a consequence of infecting a cell, transform a normal cell into an abnormally proliferating cell. A “tumor virus” as used herein may, for example, be a DNA tumor virus or an RNA tumor virus. Examples of “tumor viruses” include simian virus 40, murine or human polyoma virus, parvovirus, papilloma virus, herpes virus, and primate adenovirus.

“BMD-13 T-HR mutant virus,” as used herein, refers to a T-HR mutant virus, containing an alteration in the common N-terminus of any of the polyoma T antigens. For example, this alteration may be a single amino acid substitution in the second position of any of the polyoma T antigens, e.g., an Aspartic Acid to Asparagine (D to N) substitution, or the deletion of amino acids 2-4 of any of the polyoma T antigens. In addition, the alteration may be present in all polyoma T antigens. A “BMD-13 T-HR mutant virus” is able to replicate and disseminate in abnormally proliferating cells, for example, BNL cells, a carcinogen-induced mouse liver cell line, but has a reduced ability to replicate and disseminate in primary, normal cells, for example, baby mouse kidney (BMK) cells. In addition, the abnormally proliferating cells may have a proliferative disease-associated alteration in a Taz nucleic acid sequence, in a nucleic acid sequence affecting expression of a protein that a Taz gene product interacts with, or in a nucleic acid sequence encoding a component of a signaling pathway involving Taz. Examples of proteins that interact with Taz gene products include ras-GTPase-activating protein SH3-domain binding protein (G3BP), Nucleolin, Vesicle Associated Protein 1, and Death Inducer with SAP domain (DIS).

By “a T-HR mutant target gene,” as used herein is meant any cellular gene that a virus must inactivate or activate, either directly or indirectly, to replicate and disseminate. Examples of “T-HR mutant target genes” include Taz, ras-GTPase-activating protein SH3-domain binding protein (G3BP), Nucleolin, Vesicle Associated Protein 1, or Death Inducer with SAP domain (DIS) nucleic acid sequence. In one embodiment, these nucleic acid sequences are mammalian. In more desirable embodiments the nucleic acid sequences are murine or human nucleic acid sequences.

By a “Taz polypeptide having wild-type activity” is meant a Taz polypeptide that reduces the ability of a BMD-13. T-HR mutant to replicate and disseminate in a cell. The reduction in the ability of a BMD-13 T-HR mutant to replicate and disseminate may be measured by determining the number of cells infected with the BMD-13 T-HR mutant that survive in presence of a “Taz polypeptide having wild-type activity” in comparison to the number of cells infected with the BMD-13 T-HR mutant that survive in the absence of a “Taz polypeptide having wild-type activity,” where an increase in the number of cells surviving is indicative of a reduction in ability of the BMD-13 T-HR mutant to replicate and disseminate. In one embodiment, the reduction in the ability of a BMD-13 T-HR to replicate and disseminate is at least 25%. In more desirable embodiments, the reduction in the ability of a BMD-13 T-HR mutant to replicate and disseminate is at least 50%, 75%, 80%, 90%, or 95%. However, a “Taz polypeptide having wild-type activity” may also completely block the ability of a BMD-13 T-HR mutant to replicate and disseminate. In addition, a “Taz polypeptide having wild-type activity” may have further functions in a cell besides reducing the ability of a BMD-13 T-HR mutant virus to replicate and disseminate.

By a “DIS nucleic acid sequence” or “Death Inducer with SAP domain nucleic acid sequence,” as used herein is meant a nucleic acid sequence that is at least 40%, 50%, 60%, 70%, 75%, 80%, 85%, 90%, 95%, or 99% identical to a nucleic acid sequence provided of SEQ ID NO:1 or SEQ ID NO:3 over a region comprising at least 200, 300, 500, 750, 1000, 1500, 2000, 2500, 3000, or 3500 contiguous nucleotides. In addition, a “DIS nucleic acid sequence” may be identical to the nucleic acid sequence of SEQ ID NO:1 or SEQ ID NO:3. In desirable embodiments, a “DIS nucleic acid sequence” is a human or a mouse DIS nucleic acid sequence that is at least 75%, 80%, 85%, 90%, or 95% identical to the human DIS nucleic acid sequence of SEQ ID NO:3, or to the murine DIS nucleic acid sequence of SEQ ID NO:1, over a region encompassing at least 1000, 2000, 3000, or 3500 contiguous nucleotides, and encodes a protein which induces DNA condensation and apoptosis in mammalian cells.

By a “DIS polypeptide,” a “Death Inducer with SAP domain polypeptide,” a “DIS amino acid sequence,” or a “Death Inducer with SAP domain amino acid sequence,” as used herein is meant an amino acid sequence that is at least 30%, 40%, 50%, 60%, 70%, 75%, 80%, 85%, 90%, 95%, or 99% identical to the amino acid sequence of SEQ ID NO:2 or SEQ ID NO:4 over a region comprising at least 50, 75, 100, 200, 300, 500, 700, 900, or 1200 contiguous amino acids. In addition, a “DIS polypeptide” may be identical to the amino acid sequence of SEQ ID NO:2 or SEQ ID NO:4. In desirable embodiments, a “Death Inducer with SAP domain (DIS) polypeptide” or a “Death Inducer with SAP domain (DIS) amino acid sequence” is a human or a mouse DIS polypeptide or amino acid sequence that is at least 30%, 50%, 60%, 70%, 80%, 90%, or 95% identical to the human DIS amino acid sequence of SEQ ID NO:4, or the mouse DIS amino acid sequence of SEQ ID NO:2, over a region encompassing 5.00, 700, 900, or 1200 contiguous amino acids, and induces DNA condensation and apoptosis in mammalian cells.

By a “Taz nucleic acid sequence,” as used herein is meant a nucleic acid sequence that is at least 40%, 50%, 60%, 70%, 75%, 80%, 85%, 90%, 95%, or 99% identical to the nucleic acid sequence of GenBank Accession No. A1317016 over a region comprising at least 100, 200, 300, 400, or 500 contiguous nucleotides. In addition, a “Taz nucleic acid sequence” may be identical to the nucleic acid sequence of GenBank Accession No. A1317016. In desirable embodiments, a “Taz nucleic acid sequence” is a human or a mouse Taz nucleic acid sequence. Furthermore, a “Taz nucleic acid sequence” may also include upstream regulatory sequences.

By a “Taz polypeptide” or a “Taz amino acid sequence,” as used herein is meant an amino acid sequence that is at least 30%, 40%, 50%, 60%, 70%, 75%, 80%, 85%, 90%, 95%, or 99% identical to the amino acid sequence encoded by GenBank Accession No. A1317016 over a region comprising at least 50, 75, 100, or 150 contiguous amino acids. In addition, a “Taz polypeptide” or a “Taz amino acid sequence” may be identical to the amino acid sequence encoded by GenBank Accession No. A1317016. In desirable embodiments, a “Taz polypeptide” or a “Taz amino acid sequence” is a human or a mouse Taz polypeptide or amino acid sequence.

By a “GAP SH3 binding protein nucleic acid sequence” or “G3BP nucleic acid sequence,” as used herein is meant a nucleic acid sequence that is at least 40%, 50%, 60%, 70%, 75%, 80%, 85%, 90%, 95%, or 99% identical to a nucleic acid sequence encoding the amino acid sequence of GenBank Accession Nos. NP_{—}038744 or 7305075 over a region comprising at least 300, 500, 750, 1000, or 1200 contiguous nucleotides. In addition, a “G3BP nucleic acid sequence” may be identical to a nucleic acid sequence encoding the amino acid sequence of GenBank Accession Nos. NP_{—}038744 or 7305075. In desirable embodiments, a “GAP SH3 binding protein nucleic acid sequence” or “G3BP nucleic acid sequence” is a human or a mouse GAP SH3 binding protein nucleic acid sequence.

By a “GAP SH3 binding protein polypeptide,” a “G3BP polypeptide,” a “GAP SH3 binding protein amino acid sequence,” or a “G3BP amino acid sequence,” as used herein is meant an amino acid sequence that is at least 30%, 40%, 50%, 60%, 70%, 75%, 80%, 85%, 90%, 95%, or 99% identical to the amino acid sequence provided in GenBank Accession Nos. NP_{—}038744 or 7305075 over a region comprising at least 50, 75, 100, 200, 300, or 400 contiguous amino acids. In addition, a “G3BP polypeptide” or a “G3BP amino acid sequence” may be identical to the amino acid sequence provided in GenBank Accession Nos. NP_{—}038744 or 73.05075. In desirable embodiments, a “G3BP polypeptide” or a “G3BP amino acid sequence” is a human or a mouse G3BP polypeptide or amino acid sequence.

By a “Nucleolin nucleic acid sequence,” as used herein is meant a nucleic acid sequence that is at least 40%, 50%, 60%, 70%, 75%, 80%, 85%, 90%, 95%, or 99% identical to a nucleic acid sequence encoding the amino acid sequence of GenBank Accession Nos. AAH05460 or 13529464 over a region comprising at least 300, 500, 750, 1000, 1500, or 2000 contiguous nucleotides. In addition, a “Nucleolin nucleic acid sequence” may be identical to a nucleic acid sequence encoding the amino acid sequence of GenBank Accession Nos. AAH05460 or 13529464. In desirable embodiments, a “Nucleolin nucleic acid sequence” is a human or a mouse Nucleolin nucleic acid sequence.

By a “Nucleolin polypeptide” or a “Nucleolin amino acid sequence,” as used herein is meant an amino acid sequence that is at least 30%, 40%, 50%, 60%, 70%, 75%, 80%, 85%, 90%, 95%, or 99% identical to the amino acid sequence provided in GenBank Accession Nos. AAH05460 or 13529464 over a region comprising at least 50, 75, 100, 200, 300, 400, 500, 600, or 700 contiguous amino acids. In addition, a “Nucleolin polypeptide” or a “Nucleolin amino acid sequence” may be identical to the amino acid sequence provided in GenBank Accession Nos. AAH05460 or 13529464. In desirable embodiments, a “Nucleolin polypeptide” or a “Nucleolin amino acid sequence” is a human or a mouse Nucleolin polypeptide or amino acid sequence.

By a “Vesicle Associated Protein 1 nucleic acid sequence,” as used herein is meant a nucleic acid sequence that is at least 40%, 50%, 60%, 70%, 75%, 80%, 85%, 90%, 95%, or 99% identical to a nucleic acid sequence encoding the amino acid sequence of GenBank Accession Nos. T14150 or 7514116 over a region comprising at least 300, 500, 750, 1000, 1500, 2000, 2500, 3000, or 3500 contiguous nucleotides. In addition, a “Vesicle Associated Protein 1 nucleic acid sequence” may be identical to a nucleic acid sequence encoding the amino acid sequence of GenBank Accession Nos. T14150 or 7514116. In desirable embodiments, a “Vesicle Associated Protein 1 nucleic acid sequence” is a human or a mouse Vesicle Associated Protein 1 nucleic acid sequence.

By a “Vesicle Associated Protein 1 polypeptide” or a “Vesicle Associated Protein 1 amino acid sequence,” as used herein is meant an amino acid sequence that is at least 30%, 40%, 50%, 60%, 70%, 75%, 80%, 85%, 90%, 95%, or 99% identical to the amino acid sequence provided in GenBank Accession Nos. T14150 or 7514116 over a region comprising at least 50, 75, 100, 200, 400, 600, 800, 1000, or 1200 contiguous amino acids. In addition, a “Vesicle Associated Protein 1 polypeptide” or a “Vesicle Associated Protein 1 amino acid sequence” may be identical to the amino acid sequence provided in GenBank Accession Nos. T14150 or 7514116. In desirable embodiments, a “Vesicle Associated Protein 1 polypeptide” or a “Vesicle Associated Protein 1 amino acid sequence” is a human or a mouse Vesicle Associated Protein 1 polypeptide or amino acid sequence.

By “reduced ability to replicate and disseminate,” as used herein is meant a reduction in the ability of a mutant virus, for example, a mutant DNA tumor virus, to replicate and disseminate, relative to a wild-type virus in the same type of cell, of at least 50%, 60%, 70%, 80%, 90%, 95%, 99%, or the complete inability to replicate or disseminate. In one desirable embodiment, the ability to replicate and disseminate is reduced by at least 90%. In more desirable embodiments, the ability to replicate and disseminate is reduced by at least 95% or 99%.

By a compound that “modulates the protein level” or “modulates the nucleic acid level” is meant a compound that increases or decreases protein or nucleic acid level of a specific protein or nucleic acid in a cell or a cell extract. For example, such a compound may increase or decrease RNA stability, transcription, translation, or protein degradation. It will be appreciated that the degree of modulation provided by a modulatory compound in a given assay will vary, but that one skilled in the art can determine the statistically significant change (e.g., a p-value ≦0.05) in the level of the specific protein or nucleic acid affected by a modulatory compound. In desirable embodiments, the protein or nucleic acid is a Taz, ras-GTPase-activating protein SH3-domain binding protein (G3BP), Nucleolin, Vesicle Associated Protein 1, or Death Inducer with SAP domain (DIS) amino acid or nucleic acid sequence.

“Alteration,” when used herein in reference to a gene, refers to a change in the coding or regulatory nucleic acid sequence or a modification of the nucleic acid sequence, for example, DNA methylation of the promoter region. The change in the coding or regulatory nucleic acid sequence may include, for example, an insertion, a deletion, or a substitution of one or more nucleic acids, as well as an inversion or a duplication. “Alteration,” when used herein in reference to a polypeptide refers to a change in the amino acid sequence. Such a change may be, for example, a substitution, a deletion, or an insertion.

“Genetic lesion,” as used herein, refers to a nucleic acid change. Examples of such a change include a single nucleic acid change as well as a deletion or an insertion of one or more nucleic acid. However, a genetic lesion can also include a duplication or an inversion. In addition, a genetic lesion may be a naturally-occurring polymorphism, for example, one that predisposes an organism carrying the polymorphism to acquiring a proliferative disease.

“Loss of heterozygosity,” as used herein, refers to a nucleic acid sequence that is homozygous for the same locus on a chromosome. For example, the normal copy of a gene is lost and both copies of the gene in a cell are mutant. A loss of heterozygosity may occur due to a structural deletion in the normal gene in the chromosome carrying this gene. Alternatively, a loss of heterozygosity may be due to recombination between the mutant and the normal gene, followed by formation of a daughter cell homozygous for the deleted or inactivated (i.e., mutant) gene. A loss of heterozygosity may also result from a loss of the chromosome with the normal gene and a duplication of the chromosome with the mutant gene. A loss of heterozygosity may be determined by standard methods in the art, for example, by using Southern blots, by sequencing, or by PCR analysis (See, for example, Debelenko et al., Hum. Mol. Genet. 6:2285-2290, 1997; and Emmert-Buck et al., Cancer Res. 55:2959-2962, 1995).

“Polymorphism,” as used herein, refers to an alteration in a nucleic acid sequence, for example, a gene, that may result in a codon change.

“Modification of function,” as used herein, refers to a change in the function of the protein. Such a change can, for example, result in the partial or complete loss of function, but it can also result in a gain of function or a new function.

“Knockout,” as used herein, refers to an alteration in the sequence of a specific gene that results in a decrease of function of that gene. In desirable embodiments, the alteration results in undetectable or insignificant expression of the gene and in a complete or partial loss of function. Furthermore, the disruption may be conditional, e.g., dependent on the presence of tetracycline. Knockout animals may be homozygous or heterozygous for the gene of interest. In addition, the term knockout includes conditional knockouts, where the alteration of the target gene can occur, for example, as a result of exposure of the animal to a substance that promotes target gene alteration, introduction of an enzyme that promotes recombination at the target gene site (e.g., Cre in the Cre-lox system, or FLP in the FLP/FRT system), or any other method for directing target gene alteration.

As used herein, “modulates proliferation” refers to any change in the proliferation of a cell, when compared to a control cell of the same type. For example, this term can be used to describe an increase or a decrease in the rate of cell division. In addition, a modulation of proliferation may refer to a normally quiescent cell entering into the cell cycle or a normally dividing cell ceasing to enter into the cell cycle.

By a “substantially pure polypeptide” is meant a polypeptide (for example, a Sal2 polypeptide) that has been separated from components that naturally accompany it. Typically, the polypeptide is substantially pure when it is at least 60%, by weight, free from the proteins and naturally-occurring organic molecules with which it is naturally associated. Preferably, the preparation is at least 75%, more preferably at least 90%, and most preferably at least 99%, by weight, a Sal2 polypeptide. A substantially pure Sal2 polypeptide may be obtained, for example, by extraction from a natural source (for example, a mammalian cell); by expression of a recombinant nucleic acid encoding a Sal2 polypeptide; or by chemically synthesizing the protein. Purity can be measured by any appropriate method, for example, column chromatography, polyacrylamide gel electrophoresis, or by HPLC analysis.

By “isolated DNA” is meant DNA that is free of the genes which, in the naturally-occurring genome of the organism from which the DNA of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. It also includes a recombinant DNA that is part of a hybrid gene encoding additional polypeptide sequence.

By a “candidate compound” or “test compound” is meant a chemical, be it naturally-occurring or artificially-derived, that is surveyed for its ability to modulate cell proliferation, by employing one of the assay methods described herein. Candidate compounds may include, for example, peptides, polypeptides, synthetic organic molecules, naturally-occurring organic molecules, nucleic acid molecules, and components thereof.

By “high stringency hybridization conditions” is meant, for example, hybridization at approximately 42° C. in about 50% formamide, 0.1 mg/ml sheared salmon sperm DNA, 1% SDS, 2×SSC, 10% Dextran sulfate, a first wash at approximately 65° C. in about 2×SSC, 1% SDS, followed by a second wash at approximately 65° C. in about 0.1×SSC. Alternatively, “high stringency hybridization conditions” may include hybridization at approximately 42° C. in about 50% formamide, 0.1 mg/ml sheared salmon sperm DNA, 0.5% SDS, 5×SSPE, 1× Denhardt's, followed by two washes at room temperature in 2×SSC, 0.1% SDS, and two washes at between 55-60° C. in 0.2×SSC, 0.1% SDS.

The tumor host range selection procedure described herein has significant advantages over genetic screens and biochemical approaches used in the past to identify viral functions and to elucidate aspects of the interaction between virus and host. For example, previous studies using conditional lethal mutants of the polyoma viruses failed to uncover the large T antigen function involving interaction with mSal2 despite the fact that this interaction is essential for virus growth both in vitro (e.g., in tissue culture) and in vivo (e.g., in the mouse). In contrast to the directed search for host range mutants based on complementation with integrated viral genes (Benjamin, Proc. Natl. Acad. Sci. U.S. 67:394-399 (1970)), the ‘tumor host range’ selection procedure of the invention is an undirected search utilizing non-polyoma transformed or tumor derived cells. Selection of virus mutants is therefore unbiased except for the possibility of being conditional on the transformed state of the particular permissive host being used. Thus, the inventive strategy can lead to the identification of viral functions and cellular targets not revealed by conventional genetic screens or co-immunoprecipitation.

Furthermore, the methods of the invention also have a particular advantage over standard chemotherapy treatments, and the like, in that they are specific for cells with a proliferative disease. Therefore, one would expect this type of therapy to have fewer toxic side effects than the chemotherapeutic agents used today.

### BRIEF DESCRIPTION OF THE DRAWINGS

FIG. 1 is a series of photographs depicting the growth of wild-type polyoma virus and the TMD25 virus on host cells.

FIG. 2A is sequence comparison between a region of the wild-type and TMD25 polyoma virus large T antigen nucleic acid and amino acid sequences and shows the 20 bp sequence duplication responsible for the TMD25 mutation.

FIG. 2B is a schematic diagram of various mSal2 clones obtained in a yeast two-hybrid assay, as well as a series of images showing the interaction of a mSal2 clone with wild-type polyoma virus large T antigen protein and the lack of interaction with TMD25 virus large T antigen protein in a yeast two-hybrid assay.

FIG. 2C is a series of large T antigen amino acid sequences and shows the deletion analysis of the TMD25 mutant.

FIG. 3A is a schematic diagram showing the regions of the mSal2 gene used to develop antibodies.

FIG. 3B is a series of Western blots using protein from mouse and human cells that show the mSal2 gene product to be p150^{sal2}.

FIG. 3C is a series of Western blots of extracts from human 293 and U2OS cells that was first probed with an antiserum against the mSal2 carboxyl-terminus. The filter was then stripped and re-probed with an antibody against the mSal2 amino-terminus.

FIG. 4A is a picture of a protein gel showing that mSal2 binds to wild-type polyoma virus, but not to TMD25 large T protein in vitro.

FIG. 4B is a series of protein gels showing the interaction of mSal2 and wild-type, but not of mSal2 and TMD-25 mutant, large T protein in transfected 3T3 cells. These results are confirmed in BMK cells infected with wild-type polyoma virus and with TMD25 mutant virus.

FIG. 5A is a series of images showing the failure of TMD25 to replicate in newborn mice.

FIG. 5B is a series of Southern blots showing that TMD25 fails to replicate in BMK cells and that p150^{sal2} represses viral origin replication.

FIG. 5C is a Southern blot showing that inhibition of replication by mSal2 requires the large T interaction domain and can be overcome by wild-type, but not mutant large T antigen.

FIG. 6 is a Western blot showing mSal2 expression in various mouse tissues.

FIG. 7 is a Western blot showing hSal2 expression in human ovarian tumors.

FIG. 8 is a Western blot showing expression of p150^{sal2} in human 293 cells.

FIG. 9 is a series of images showing immunostaining of p150^{sal2} in human ovary tissue (A) and in ovarian tumors (B).

FIG. 10A is a series of images and a bar graph showing that p150^{sal2} suppresses growth of human ovarian tumor cells, which is indicated by a reduction in BrdU incorporation in p150^{sal2} transfected cells.

FIG. 10B is a series of images showing a colony reduction assay that indicates that cells transfected with p150^{sal2} are less viable than control transfected cells.

FIG. 11 is an agarose gel showing that the 73S allele is lost in some ovarian tumors.

FIG. 12 is a schematic diagram of two embodiments of the BMD-13 T-HR mutant virus.

FIG. 13 is a schematic diagram of the murine Taz protein.

FIG. 14 is a picture of a Western blot showing that mTaz binds to the T antigens.

FIG. 15 is a picture of a Western blot showing that human Taz binds to the SV40 large T antigen in vivo.

FIG. 16 is a series of protein gels showing that amino acids 2-4 of murine Taz are essential for its interaction with middle (panel A) and small (panel B) T antigens.

FIG. 17 is a picture of a series of Western blots showing that Taz binds to PP2A and Src.

FIG. 18 is a picture of a protein gel showing that the phosphorylation state of murine Taz changes as a result of polyoma virus infection.

FIG. 19 is a series of scanned images showing the intracellular localization of murine Taz and how it is altered in response to wild-type or mutant polyoma virus infection.

FIG. 20 is a picture of a series of Western blots showing that the small T antigen increases binding of Taz to the large T antigen.

FIG. 21 is a non-limiting model of Taz function during polyoma virus infection.

FIG. 22 is the sense (SEQ ID NO:1) and the antisense strand of the murine DIS nucleic acid sequence as well as the corresponding amino acid sequence (SEQ ID NO:2).

FIG. 23 is the murine DIS nucleic acid sequence (SEQ ID NO:1).

FIG. 24 is the murine DIS nucleic acid sequence (SEQ ID NO:1) and the amino acid sequence encoded by the open reading frame of murine DIS (SEQ ID NO:2).

FIG. 25 is the sense (SEQ ID NO:3) and the antisense strand human DIS nucleic acid sequence as well as the corresponding amino acid sequence (SEQ ID NO:4).

FIG. 26 is the human DIS nucleic acid sequence (SEQ ID NO:3).

FIG. 27 is the human DIS nucleic acid sequence (SEQ ID NO:3) and the amino acid sequence encoded by the open reading frame of human DIS (SEQ ID NO:4).

FIG. 28 is a picture of a Southern blot showing that polyoma virus replication is inhibited by mTaz.

FIG. 29 is scanned image showing Tet induced TAZ expression (A) and a graph showing that TAZ inhibits origin replication (B).

FIG. 30 is a Southern blot (A) showing a loss of heterozygosity in DIS in ovarian tumors, and a Western blot (B) showing a lack of DIS expression in ovarian tumors.

FIG. 31 is a series of images showing that murine DIS induces apoptosis.

FIG. 32 is a series of Western blots showing that DIS, PARP, and LaminB are cleaved in BMK and HeLa cells upon induction of apoptosis by staurosporine (A), and that caspase-3 and caspase-8 inhibitors can inhibit DIS cleavage (B).

FIG. 33 is a series of schematic diagrams showing the location of several caspase-3 and caspase-8 cleavage sites in human (top) and murine (bottom) DIS.

FIG. 34 is a series of Western blots showing that DIS is sensitive to caspase-3 cleavage and that the first caspase-3 site, at amino acid 691 of human DIS and at amino acid 689 of murine DIS, is used for cleavage.

FIG. 35 is a schematic diagram showing the genomic organization of exon 2 of TAZ, and structure of the targeting vector used to generate TAZ knock out mice.

FIG. 36A shows a series of immunoblots and a colony reduction assay demonstrating the effects of p150^{Sal2} expression on the growth of SKOV-3 cells. The top left panel represents p150 expression by Western blot analysis of human ovarian surface epithelial (HOSE) cells and three other ovarian tumor cell lines. The lower left panel shows p150 expression by Western blot analysis in various cells with transfected empty vector (Vector) or a p150^{Sal2} expression construct (p150). Tubulin is used as a loading control. The right panel demonstrates that neomycin resistant colonies were reduced in SKOV-3 and RumGS cells transfected with a p150^{Sal2} expression construct (p150) compared to empty vector (Vector) but not in HOSE or IGR-OV-1 cells.

FIG. 36B are three photographs and a graph representative of a BrdU incorporation assay. SKOV-3 cells were transfected with either empty vector (Vector), constructs express p150 (p150), or p53 (p53) and incubated with BrdU containing medium. EGFP construct was cotransfected with each experiment to identify the transfected cells. The cells are then stained for BrdU and DAPI. Results are presented as percent of BrdU negative (BrdU⁻) cells among total transfected cells (positive for co-transfected EGFP).

FIG. 36C shows a series of photographs and a bar graph. In the top, left panel, DAPI staining pattern of apoptotic SKOV-3 cells induced by actinomycin D were confirmed by TUNEL assay. In the bottom, left panel, SKOV-3 cells were transiently transfected with p150 or empty vector together with GFP and stained with DAPI. Arrows indicate cells that are p150 transfected and also apoptotic. Quantification is shown on the right as percent of apoptotic cells among all transfected cells.

FIG. 37A is an immunoblot showing p150^{Sal2} expression in normal HOSE cells, pooled neomycin resistant SKOV-3 cells transfected with empty vector (SK-Vector), and three independently derived SKOV-3 clones with various p150^{Sal2} expression levels (SK-P150-1, 2, 3).

FIG. 37B is a series of photographs showing the reduction of anchorage independent growth in SK-P150 cells with restored p150^{Sal2} expression compared to SK-Vector cells.

FIG. 37C is a series of photographs showing a SCID mouse inoculated with SKOV-3 cells with stably integrated empty vector (SK-Vector) and SKOV-3 cells with stably integrated p150 expression construct (SK-P150-1).

FIG. 37D is a series of graphs showing the weight of tumors from SK-Vector cells and from three independent SKOV-3 clones with human p150 (SK-P150, SK-P150-2 and SK-P150-3).

FIG. 37E shows a series of photographs and a bar graph showing the level of apoptosis in SK-Vector and SK-P150 induced tumors. The tow photographs represent TUNEL staining of sections of SK-vector and SK-P150-1 tumors. Arrows indicate TUNEL positive cells. On the right, apoptotic cells in SK-Vector and SK-P150-1 tumors are quantified as shown in the bar graph. The results represent the average of two independent tumors of each type, two sections of different areas and three random fields of each section.

FIG. 37F shows a series of photographs and a bar graph showing the level of mitosis in SK-Vector and SK-P150 tumors. H&E staining of sections of SK-Vector and SK-P150 tumors are shown on the left. Arrows indicate mitotic cells. The bar graph shows quantification of mitotic cells in SK-Vector and SK-P150 tumors. The sampling is the same as in FIG. 37E.

FIG. 38A is a series of immunoblots and a bar graph. Left: Western blot for p21 in empty vector transfected SKOV-3 cells (SK-Vector) and three independent SKOV3-p150^{Sal2} clones (SK-P150, SK-P150-2, SK-P150-3). α-tubulin was used as a loading control. Right: Quantification of p21 levels in SK-Vector and SK-P150 clones.

FIG. 38B is a series of photographs representing semi-quantitative RT-PCR comparison of p21 RNA level using either SK-Vector or SK-P150 total RNA. G3PDH (glyceraldehyde-3-phosphate dehydrogenase) transcript was used as an internal control. Relative levels of p21 transcripts of SK-Vector and SK-P150 were assessed by comparing the 0.6 kb p21 RTPCR products of same cycle numbers within the linear range of amplification (34 to 40 cycles). The quantification is normalized by the levels of transcripts of G3PDH within its own linear range of amplification (28-34 cycles) as shown in the bar graph.

FIG. 38C show a series of bar graphs The top graph shows that p150^{Sal2} stimulates p21 promoter activity. Increasing amounts of a human p150 cDNA expression construct (p150) were co-transfected into SKOV-3 cells along with a luciferase reporter driven by a 2.7 kb human p21 promoter (P21-Luc). An empty vector (Vector) and an unrelated (thymidine kinase) promoter-luciferase construct (PG-TK-Luc) were used as controls. Stimulation of p21 promoter activity by p150^{Sal2} is presented as fold increase in luciferase activity over that of the vector control normalized by the activity of co-transfected β-galactosidase. The bottom graph shows the effect of p150 and p53 on P21-luc induction. An additive effect was seen when p150^{Sal2} and p53 was co-transfected (p50+p53), compared to each of p150 (p150) and p53 (p53) alone.

FIG. 38D shows a schematic diagram, a photograph of a colony reduction assay, a bar graph, and a series of immunoblots. The schematic diagram shows p150^{Sal2} protein and the location of deleted zinc finger motifs in p150Δ3 (deletion of aa631-711) and p150Δ2 (deletion of aa911-956). The top right panel shows a photograph of a colony reduction assay using empty vector (Vector), p150^{Sal2} (p150), and Zinc finger domain deletions of the DNA binding triple zinc finger (p150Δ3) or the T antigen interacting last zinc finger pair (p150Δ2) in SKOV-3 cells. The bar graph demonstrates the induction of p21-luc activities by vector, p150, p150Δ3, and p150Δ2 in SKOV-3 cells. The series of immunoblots show the expression level of p150^{Sal2} in SKOV-3 cells transfected with Vector or p150, p150Δ3, and p150Δ2.

FIG. 39A shows a series of photographs of mock transfected (Mock) or p150 siRNA transfected (p150siRNA) HOSE cells cultured in BrdU containing medium for 20 hours. Nuclei with BrdU incorporation were stained with anti-BrdU antibody (red). Nuclei were stained with DAPI (Blue). The obtained results were quantified by counting BrdU positive cells in 4 random fields as percentage of total cells.

FIG. 39B shows a series of immunobltos representing a Western blot analysis of knockdown of p150^{Sal2} by siRNA (P150-siRNA) reduced p21 level in HOSE cells compared to mock transfected cells (Mock). The same blot was stripped and reprobed for α-tubulin as the loading control.

FIG. 40A is a schematic diagram showing the 2.7 kb human p21 promoter region and its digestion fragments used in the protein DNA immunoprecipitation (Mckay) assay (sizes in base pairs are given under each fragment).

FIG. 40B is a series of photographs representing immuno-precipitation of Ava I, Sty I or Sty I/Msp I double digested fragments of p21 promoter by p150 antibody (AntiP150) with preimmune serum (Pre-1 mm) as a control. Wild type (Wt) p150 or p150 with deleted putative DNA binding domain (Δ3) is from either P19 cell extract (Cell) or the in vitro translation (IVT) product of cloned human cDNA. Empty cloning vector was used as a negative control for in vitro translated p150 in the immunoprecipitation of Sty I/MspI fragments.

FIG. 40C shows a schematic diagram and graph representing showing that a p150 responsive cis-acting element is located 1.4 kb upstream of human p21 promoter corresponding to the distal p150^{Sal2} binding region. The luciferase assay is conducted in SKOV-3 cells to assess responsiveness of various p21 promoter deletion constructs. The fold induction represents the corresponding luciferase activity with co-transfected p150^{Sal2} over that co-transfected with empty vector. The p53 binding sites are shown as landmarks.

FIG. 40D is a series of photographs showing that p150 binds p21 promoter region in vivo. Chromatin Immuno-precipitation was performed using serum against p150 (Anti-p150) to precipitate chromatin cross-linked with p150 in p19 cells and amplified using p21 promoter specific primers (p21) and primers for glyceraldehyde-3-phosphate dehydrogenase (GAPD). The same sets of primers were also used for PCR amplification from Pre-immune serum precipitated chromatin (PreImm) and total input chromatin extract (Total) as controls.

FIG. 41 is a photograph showing the suppression of HPV-16 origin of replication by hSal2 in C33A cells.

FIG. 42 is a series of immunoblots showing p150^{Sal2} expression in various cancers.

### DETAILED DESCRIPTION OF THE INVENTION

The present invention provides a method for identifying genes that play a role in cancer as well as methods for diagnosing and treating patients who have cancers involving these genes.

For example, the invention features the use of Sal2 nucleic acids and proteins in methods for treating patients having proliferative disorders, such as cancers, involving mutations in a Sal2 nucleic acid sequence and in the protein that it encodes. In addition, these treatment methods may also be used for patients having a mutation in a nucleic acid sequence encoding a protein that interacts with Sal2 or that function in a signaling pathway involving Sal2. Furthermore, Sal2 may be used as an anti-viral agent that interferes with the ability of a DNA tumor virus to replicate and disseminate in a cell.

Host Range Selection of Viruses

The present invention describes the use of tumor host range mutant viruses (T-HR mutants) that are capable of replicating in abnormally proliferating cells but not in normal cells. Therefore, these viruses are useful for identifying genes altered in abnormally proliferating cells. T-HR mutants generally have a mutation that causes a modification of function of the protein encoded by that gene. These mutations typically lie in the transforming genes of the DNA tumor viruses and are usually activators of cellular proto-oncogenes or inactivators of tumor suppressor genes. T-HR mutants may be isolated based on their ability to propagate (i.e. to replicate and disseminate) only in tumor cells that have a mutation in a cellular protein that is normally targeted by a viral transforming protein. However, while permissive hosts for T-HR mutants could fail to express, or carry a mutation in, the cellular target itself, these hosts, or other cancer cells, may alternatively be defective in some interacting partner or effector of the target gene. Examples of the latter possibility are, for example, permissive hosts for polyoma or adenovirus mutants that are defective in binding pRb or p53, despite expressing these tumor suppressors themselves, as a result of defects in INK4A gene products which impinge on pRb and p53 (Freund et al., J. Virol. 68:7227-7234, 1994; Harvey and Levine, Genes & Dev. 5:2375-2385, 1991; Yang et al., Cancer Res. 61:5959-5963, 2001; McCormick, Oncogene 19:6670-6672, 2000; Linardopoulos et al., Cancer Res. 55:5168-5172, 1995).

The methods of the invention have been applied to a ‘tumor host range’ selection procedure using the polyoma virus as a tool to search for new interactions of viral proteins, e.g., T antigens, with cellular proteins. The rationale behind this approach is based on the idea that genetic changes in tumor cells resulting in a modification of function of the cellular protein can provide the basis for a search to uncover new viral functions and interactions with cellular targets. In principle, ‘Tumor host range’ selection could reveal mutations in other functions, e.g., VP1, 2 or 3 involving interactions with receptors or the cellular machinery involved in virus uptake, uncoating or transport to the nucleus, or even in some aspect of virus assembly, or enhancer mutations that lead to alterations in enhancer function.

For example, alterations in yet unknown targets of viral genes might occur in spontaneous tumors or non-virally transformed cells. This suggests a rationale for isolating T-HR mutants based on modification of function in cancer cells. Mutants selected to grow in tumor cells, but not in normal cells, are useful for identifying new viral gene functions and their cellular targets. Targets identified in this way may include products of tumor suppressor genes or proto-oncogenes or any factor expressed in normal cells, which the virus must inactivate in order to propagate, but that is no longer expressed in tumor cells.

T-HR mutants may be identified using the methods outlined in Table 1. Briefly, T-HR mutants may be identified using the following steps: (a) providing a wild-type viral DNA; (b) introducing random mutations in the wild-type viral DNA, thereby obtaining a collection of uncharacterized mutant viruses; (c) infecting abnormally proliferating cells with the obtained collection of mutant viruses to amplify mutant viruses; (d) selecting mutant viruses from the collection that have the ability to proliferate in abnormally proliferating cells by plaque isolation; (e) infecting normally proliferating cells with the selected mutant viruses; (f) identifying mutant viruses from step (e) that do not proliferate in the normally proliferating cells, thereby identifying THR mutant viruses.

Identification of Tumor Suppressor and Oncogenes Using T-HR Mutants

The utility of the T-HR mutant based approach for identifying new genes involved in the susceptibility to proliferative diseases is shown by the identification of mSal2. The use of a T-HR mutant coupled with the power of the yeast two-hybrid screen resulted in the identification of a cellular target protein. Using T-HR mutants to identify cell cycle regulatory proteins is advantageous on two levels; first, in choosing an appropriate wild-type ‘bait’ corresponding to the region altered in the mutant, and second, in enabling a counterscreen where lack of interaction with the mutant is helpful in identifying cellular target(s) relevant to the mutant phenotype and possibly also to the transformed state of the permissive host. One embodiment of the general protocol included as an aspect of the invention is outlined in Table 1 shown above.

We describe further below the use of the methods of the invention to identify a new target of large T antigen, referred to as mSal2, using T-HR mutants of the polyomavirus. First, tumor host range selection identified a host range mutant of the polyomavirus that is able to grow in certain tumor or transformed cells but not in normal cells. The mutant virus encodes an altered large T antigen protein and is defective in replication and tumor induction in newborn mice. Next, mSal2 was identified as a binding target of the polyoma virus large T antigen through a yeast two-hybrid screen. mSal2 shows no interaction with the mutant large T antigen. Specifically, the mutant virus fails to bind mSal2 and is unable to propagate or to induce most of the tumor types in the mouse that the wild-type virus typically induces.

The gene product p150^{sal2} is expressed in a number of mouse and human tissues. It is found in nuclei of germinal epithelial cells from normal human ovary but is missing or altered in ovarian carcinomas derived from these cells (Table 3). Using an antibody to mSal2 that cross-reacts with the human protein, Sal2 was shown to be expressed as a protein of approximately 150 kDa in several normal murine and human tissues. Normal human ovarian epithelial cells show strong nuclear staining with the antibody. A majority of ovarian carcinomas derived from these cells show no detectible p150^{sal2} by Western analysis and are negative by in situ immunochemistry. Some tumors display diffuse cytoplasmic, rather than nuclear, staining. (See Examples below.)

mSal2 is a zinc finger protein and a putative transcription factor that may have a role as a tumor suppressor. mSal2 is homologous to the Drosophila homeotic gene spalt and to sal homologues identified in several vertebrate species (see below). The human homologue of the Drosophila spalt gene, hSal2, has been mapped adjacent to, or overlapping with, a chromosomal region associated with a loss of homozygosity in ovarian and other cancers.

The spalt or sal gene family of transcription factors is conserved in evolution from flies to man. First identified in Drosophila, spalt is a region-specific homeotic gene which functions in specifying anterior and posterior structures in the early embryo (Kuhnlein et al., EMBO J. 13:168-179 (1994); Jurgens et al., EMBO J 7:189-196 (1988)) and also in later stages of organogenesis (Kuhnlein et al., Mech. Dev. 66:107-118 (1997); Barrio et al., Dev. Biol. 215:33-47 (1999)). spalt-related sal genes have been identified and studied in worms (Basson et al., Genes Dev. 10:1953-1965 (1996)), fish (Koster et al., Development 124:3147-3156 (1997)), frogs (Hollemann et al., Mech. Dev. 55:19-32 (1996); Onuma, Biochem. Biophys. Res. Commun. 264:151-156 (1999)), mice (Ott et al., Mech. Dev. 56:117-128 (1996); Kohlhase et al., Nat. Genet. 18:81-83 (2000)) and man (Kohlhase et al., Genomics 38:291-298 (1996); Kohlhase et al., Genomics 1:216-222 (1999); Kohlhase et al., Cytogenet. Cell Genet. 84:31-34 (1999)). In humans, a defect in the hSal1 gene underlies the multiple developmental defects seen in Townes-Brocke syndrome (Kohlhase et al., Nat. Genet. 18:81-83 (1998)). Sal proteins contain multiple Zinc fingers, which frequently occur as C₂H₂ pairs with a conserved motif (Kuhnlein et al., EMBO J. 13:168-179 (1994)). mSal2 has a structural arrangement typically seen in vertebrates with a single finger (C3H) near the amino-terminus and a cluster of three fingers (C₂H₂) considered essential for DNA binding in the middle portion of the protein (Pabo et al., Annu. Rev. Biochem. 61:1053-1095 (1992)). Like other Sal proteins, mSal2 has both glutamine-rich and proline- and alanine-rich sequences consistent with its transcriptional activator and repressor functions.

Although it has been shown in several species that Sal family transcription factors play important roles in embryonic development, downstream target genes have yet to be identified. Nevertheless, two important signaling pathways lying upstream of sal have been recognized. Regulation of spalt occurs in part through dpp, a member of the TGF-β family, which functions as a ‘gradient morphogen’ in the early Drosophila embryo (de Celis et al., Nature 381:421-424 (1996); Lecuit et al., Nature 381:387-393 (1996); Nellen et al., Cell 85:357-368 (1996)). In Medaka, Sal1 expression occurs in response to hh (hedgehog) and is downregulated through PK-A (Koster et al., Development 124:3147-3156 (1997)). The TGF-β family of polypeptides has well known inhibitory effects on epithelial cell growth and survival. Disruptions in signaling pathways initiated by TGF-β are known to occur in some cancers (Kretzschmar et al., Current Opinion in Genetics & Development 8:103-111 (1998); Serra et al., Nature Med. 2:390-391 (1996)). In particular, mutations in SMAD genes, essential mediators of signaling via TGF-β receptors, have been linked to pancreatic, colorectal, and other cancers (Eppert et al., Cell 86:543-552 (1996); Hahn et al., Science 271:350-353 (1996); Schutte et al., Cancer Res. 56:2527-2530 (1996)). Similarly, disruptions in signaling via ‘hedgehog’ ligands and their ‘patched’ receptors are important in development of basal cell carcinoma (Hahn et al., Cell 85:841-851 (1996); Johnson et al., Science 272:1668-1671 (1996); Oro et al., Science 276:817-821 (1997); Stone et al., Nature 384:129-134 (1996)).

Identification of T-HR Mutants (e.g., BMD-13 T-HR Mutant)

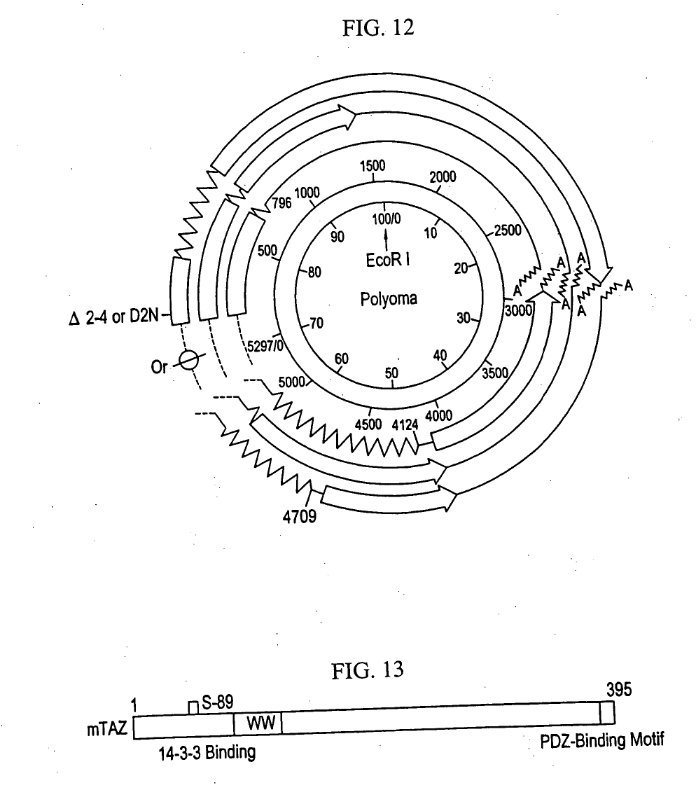

Here, we describe the isolation of TH-R mutants (e.g., BMD-13 T-HR mutant, as shown in FIG. 12) that are capable of replicating in abnormally proliferating cells, but not in normal cells. In general, permissive hosts for T-HR mutants could fail to express, or carry a mutation in, the cellular target itself, or, alternatively, these hosts, or other cancer cells, may be defective in some interacting partner or effector of the target gene or in a gene which functions in the same pathway(s) as the target gene itself (Li et al., Proc. Natl. Acad. Sci. USA 98:14,619-14,624, 2001). Examples of the latter possibility are permissive hosts for polyoma or adenovirus mutants that are defective in binding pRb or p53, despite expressing these tumor suppressors themselves, as a result of defects in INK4A gene products which impinge on pRb and p53 (Freund et al., J. Virol. 68:7227-7234, 1994; Harvey and Levine, Genes & Dev. 5:2375-2385, 1991; Yang et al., Cancer Res. 61:5959-5963, 2001; McCormick, Oncogene 19:6670-6672, 2000; Linardopoulos et al., Cancer Res. 55:5168-5172, 1995).

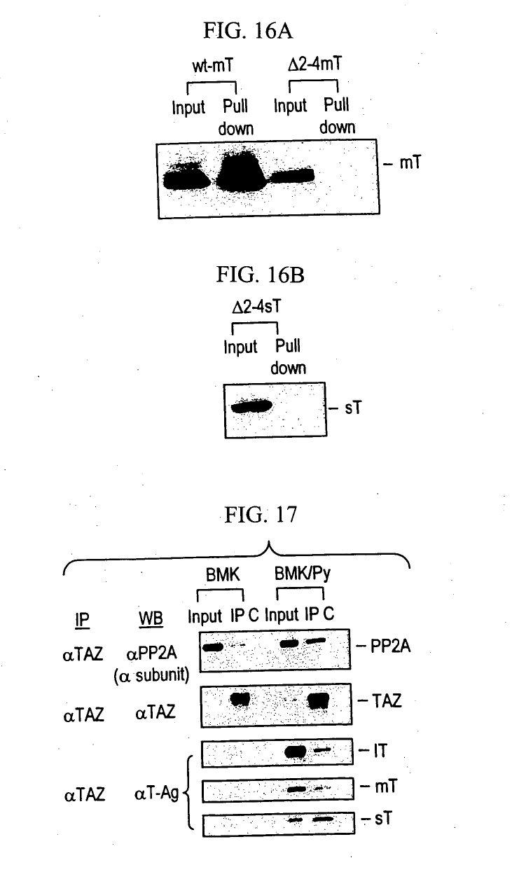

The BMD-13 T-HR mutant virus has altered T antigens that are unable to interact with a cellular Taz protein. Since T-HR mutants generally have a mutation that causes a modification of function of the protein encoded by that gene and since these mutations typically lie in the transforming genes of the DNA tumor viruses and are usually activators of cellular proto-oncogenes or inactivators of tumor suppressor genes, the Taz protein is likely to be normally targeted by the viral transforming proteins. Accordingly, a normal Taz nucleic acid or protein, like other cellular targets of DNA tumor viruses, may be used as an anti-cancer or anti-viral agent. Furthermore, the BMD-13 T-HR mutant virus may be used to identify abnormally proliferating cells, or cells that have the potential to become abnormally proliferating cells, as well as to selectively kill such cells. FIG. 16 shows that amino acids 2-4 of murine Taz are essential for its interaction with middle (panel A) and small (panel B) T antigens.

The BMD-13 T-HR mutant was isolated using the general protocol outlined above. We used the tumor host range selection procedure to identify a T-HR mutant polyoma virus (BMD-13) that is able to replicate and disseminate in BNL cells (a carcinogen-induced mouse liver tumor derived cell line), but replicates and disseminates poorly in primary baby mouse kidney (BMK) cells. The inability of this virus to propagate on normal, primary cells is due to a single amino acid substitution in all polyoma T antigens (sT, mT, and lT). We determined that the BMD-13 T-HR mutant that we isolated encodes altered T antigen proteins that have an Aspartic Acid to Asparagine (D to N) substitution at the second position of the T antigen amino acid sequences.

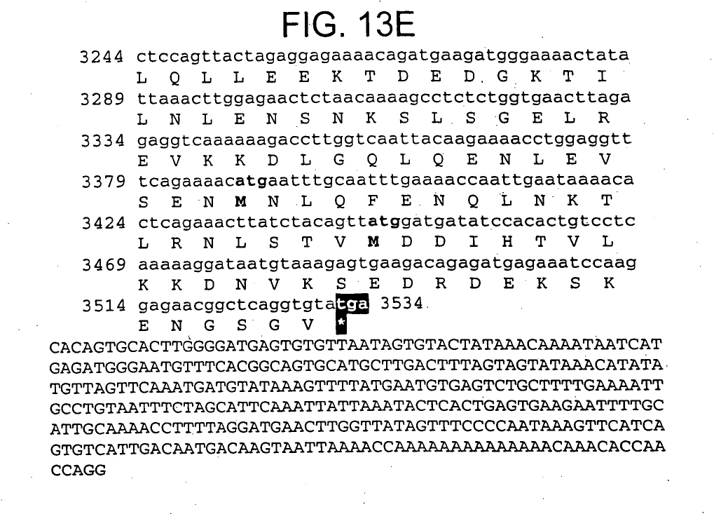

To identify the binding target for these altered T antigens, we used a “flipped” bait yeast two-hybrid system. This method involved screening a mouse cDNA library made from 17 day-old embryos with a wild-type small T (sT) protein fused to the Gal4 DNA binding domain (sT-Gal4BD). Our screen yielded a single positive clone, mTaz (transcriptional co-activator with PDZ binding motif; Kanai et al., EMBO J. 19:6778-6791, 2000; GenBank Accession No. A1317016). As shown in FIG. 13, mTaz contains a 14-3-3 binding sequence, a PDZ binding motif, and a WW domain which recognizes proline-rich regions present in known transcription factors including PEBP2α and other members of the Runx family, alterations in which have been linked to human cancer.

PDZ domains were first identified in the post-synaptic density protein PSD95, in the Drosophila tumor suppressor protein Dlg1, and in the tight junction protein ZO-1, but now have been found in at least 600 proteins. Most PDZ domain containing proteins are membrane associated, but several have been shown to reside in, or transit to, the nucleus. 14-3-3 proteins, on the other hand, generally reside in the cytoplasm. These proteins form a large, ubiquitously expressed family found in virtually all organisms, including mammals, plants, yeast, and fungi. In general, 14-3-3 proteins bind to proteins that have been phosphorylated on serine or threonine residues.

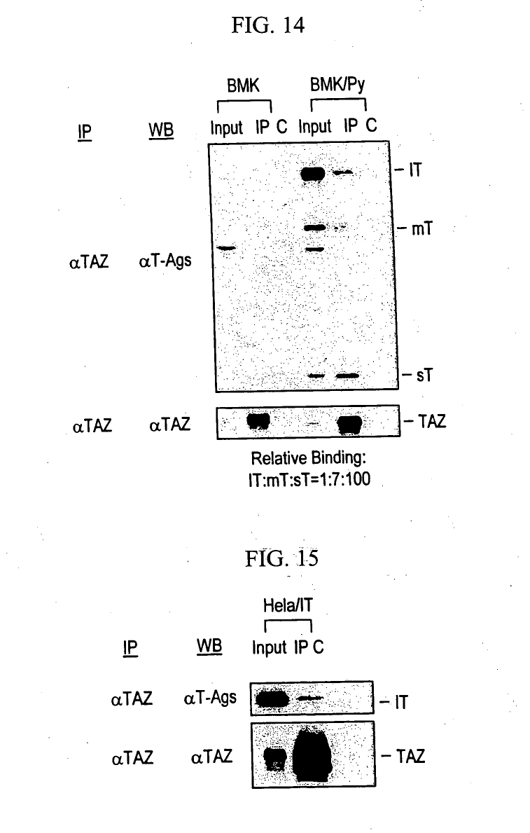

To verify the specificity of the interaction between T antigens and Taz, we immunoprecipitated Taz using a rabbit anti-Taz antibody from both uninfected and from wild-type polyoma virus infected BMK cells. We performed a Western analysis on the immunoprecipitated proteins and probed the Western blot with an anti-T antigen antibody. As is shown in FIG. 14, all three T antigens immunoprecipitated with Taz, but these proteins were present in different abundances. The relative binding efficiencies of murine Taz to the T antigens is large T: middle T: small T=1:7:100.

In addition, we confirmed the in vitro binding results by showing that human Taz interacts with the SV40 large T antigen in vivo. We transfected HeLa cells with both human Taz and SV40 large T antigen and used a rabbit anti-Taz antibody to immunoprecipitate Taz from extracts made from these cells. A Western blot of a protein gel on which these extracts were run shows that an anti-SV40 large T antigen antibody recognizes a protein of the appropriate size in the immunoprecipitated lane (FIG. 15).

Furthermore, we wanted to show that the region mutated in the BMD-13 T-HR virus interacts with Taz. We tested several N-terminal deletions and determined that a three amino acid deletion (Δ2-4) abolishes the interaction between Taz and small and middle T antigens. Not only do T antigens having the Δ2-4 fail to interact with Taz, but polyoma viruses carrying this deletion also fail to transform mammalian cells. We counted foci obtained by transfecting 1×10⁶ F111 rat fibroblast cells with 1 μg of virus genome and obtained more than 100 foci using a wild-type virus, but failed to obtain any foci with a Δ2-4 mutant polyoma virus. Accordingly, amino acids 2-4 of the T antigens are essential for transformation as well as for binding to Taz.

Proteins that Interact with TAZ

To further characterize the function of Taz, we looked at whether Taz binds to other proteins besides T antigens, in particular ones that are known to bind T antigens. In this regard, we performed another immunoprecipitation experiment and showed that Taz interacts with PP2A, a serine-threonine phosphatase known to associate with the large T antigen, as well as with c-src. Moreover, our results indicate that these interactions are increased in response to wild-type polyoma virus infection (FIG. 17).

To determine if Taz interacts with proteins other than T antigens, we used a purified anti-Taz polyclonal antibody cross-linked to Protein A in immunoprecipitation experiments. We used this antibody, as well as a normal IgG control antibody, to immunoprecipitate proteins from extracts of BMK, BNL, and P19 (embryonic carcinoma) cells. The immunoprecipitates were washed and analyzed by polyacrylamide gel electrophoresis followed by Coomassie Blue staining. The bands that differed between the anti-Taz and control lanes were cut out and subjected to standard mass spectrometry techniques. From the mass spectrometry results, we identified four additional proteins that interact with TAZ, ras-GTPase-activating protein SH3-domain binding protein (GAP SH3 binding protein or G3BP) (GenBank Accession Number 7305075), Nucleolin (GenBank Accession Number 13529464), Vesicle Associated Protein 1 (GenBank Accession Number 7514116), and Death Inducer with SAP domain (DIS).

G3BP is also known as human DNA helicase VIII. Like other proteins that are part of the Ras signal transduction pathway, G3BP is overexpressed in human tumors. In addition, this protein regulates S phase entry (Guitard et al., Cancer Lett. 162:213-221,2001; Costa et al., Nucleic Acids Res. 27:817-821, 1999; and Tocque et al., Cell Signal 9:153-158, 1997).

Nucleolin, which is also known as human DNA helicase IV, is a multifunctional major nucleolar phosphoprotein. Nucleolin acts as an RNA binding protein, an autoantigen, a transcriptional repressor, and a switch-region targeting factor. In addition, nucleolin exhibits autodegradation, DNA and RNA helicase activities, and DNA-dependent ATPase activity (Srivastava et al., FASEB J. 13:1911-1922, 1999; Tuteja and Tuteja, Crit. Rev. Biochem. Mol. Biol. 33:407-436, 1998; and Ginisty et al., J. Cell Sci. 112:761-772, 1999).

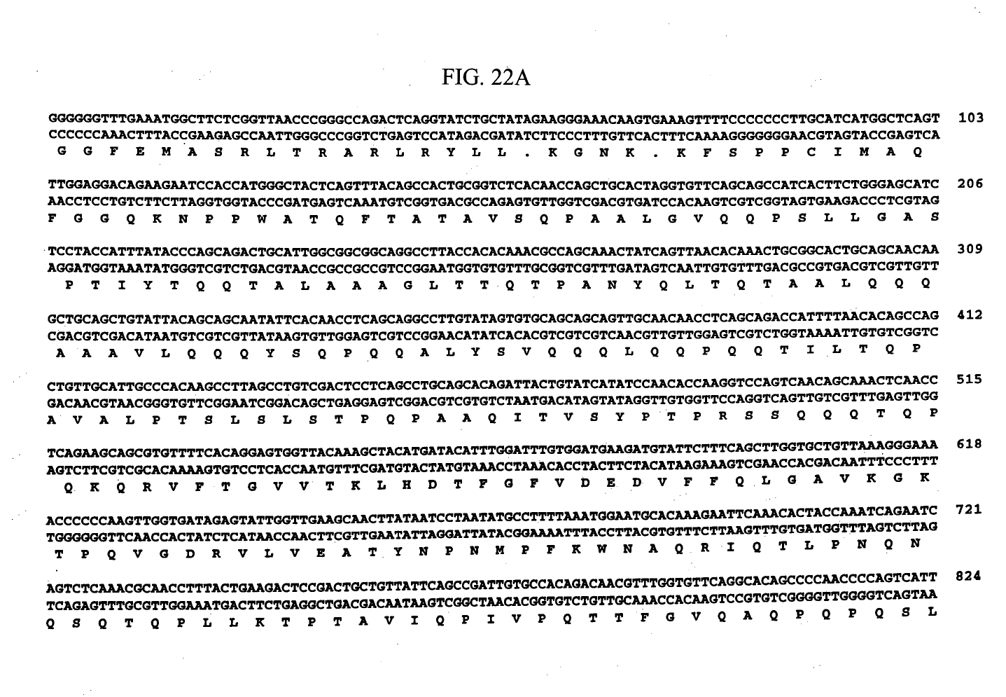

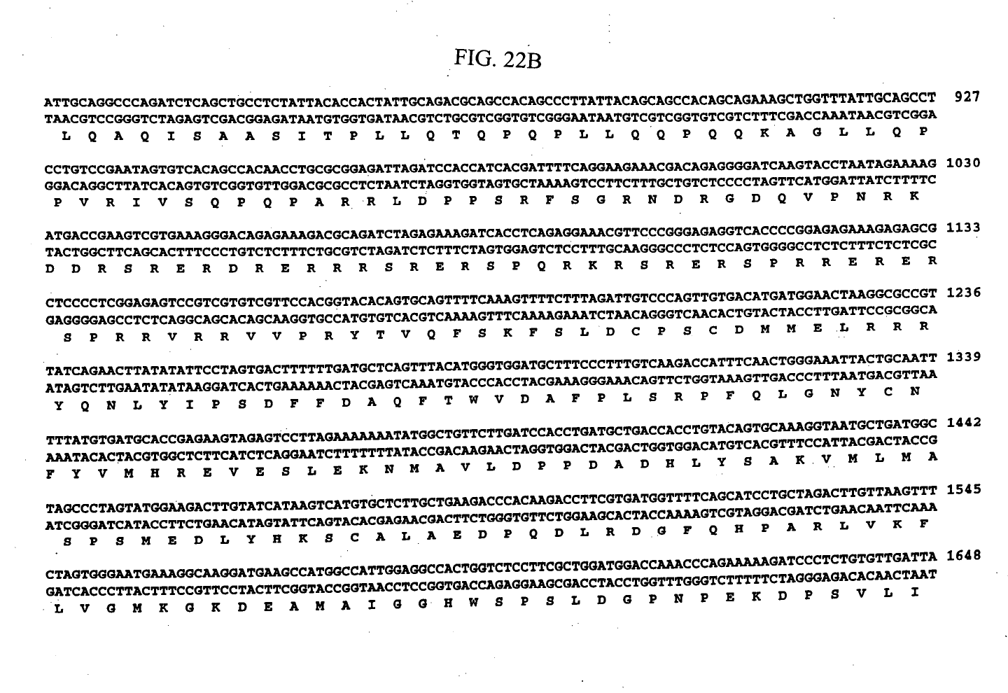

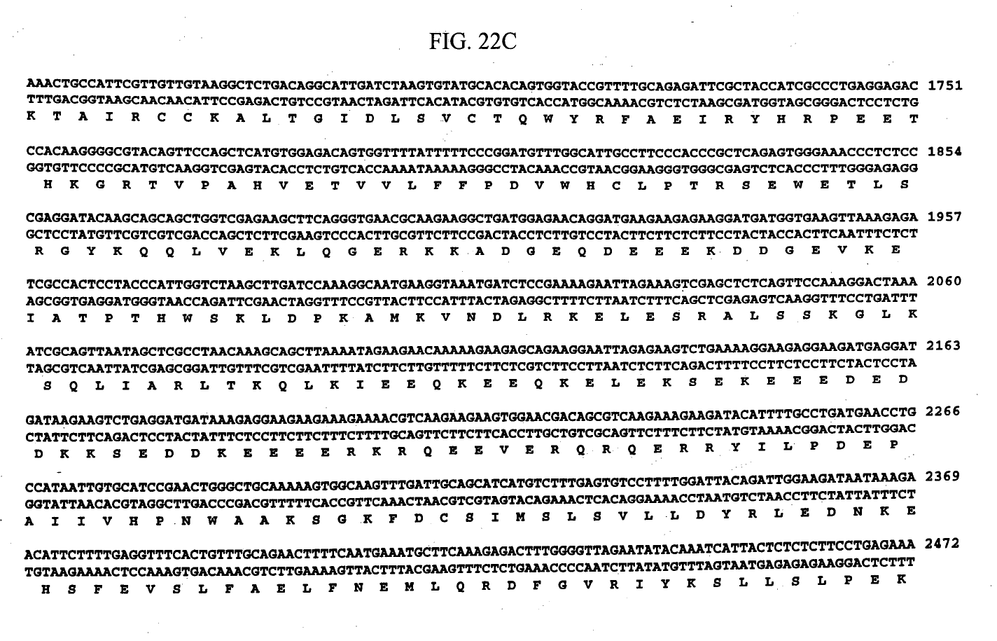

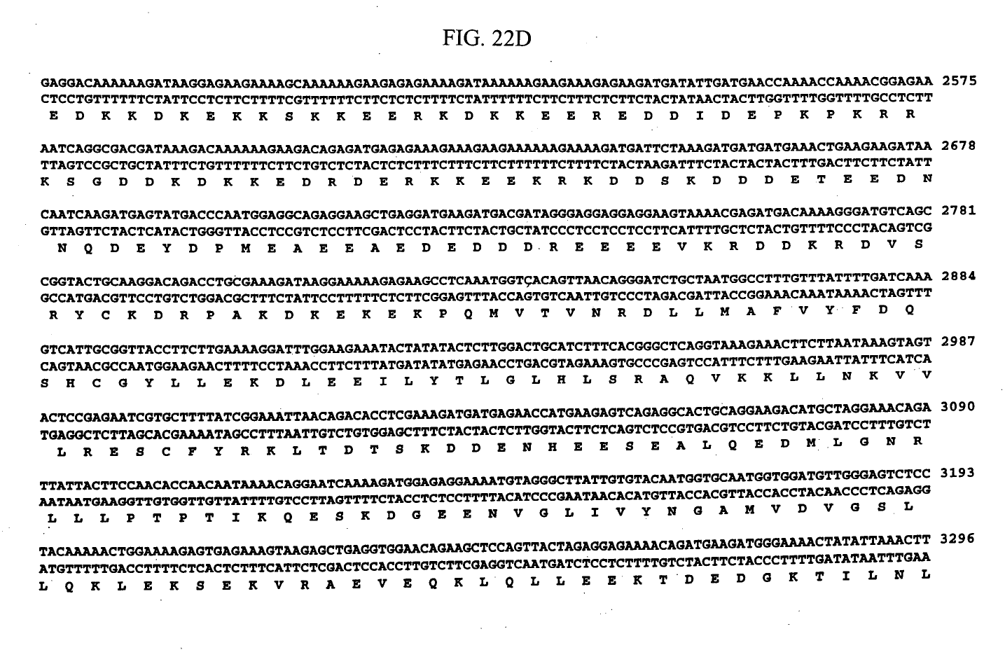

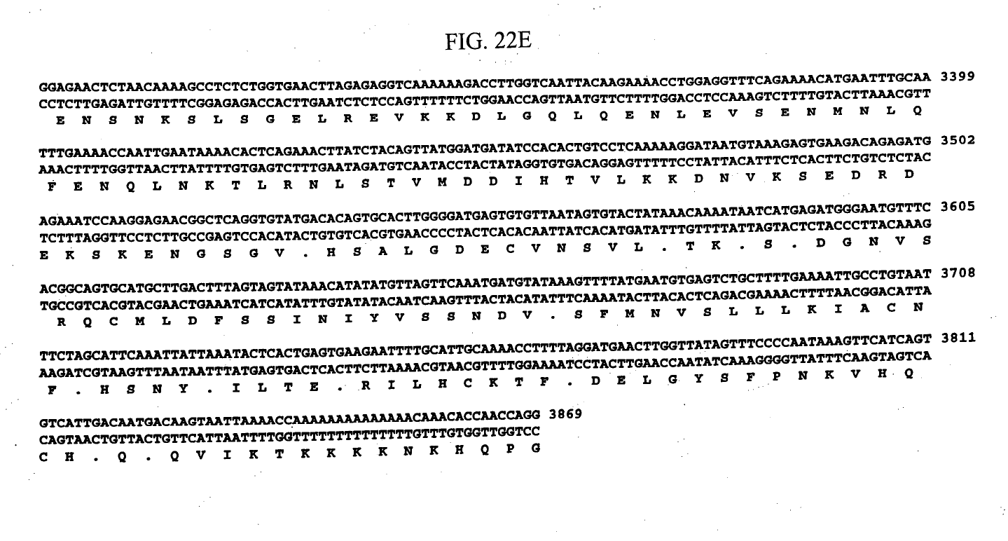

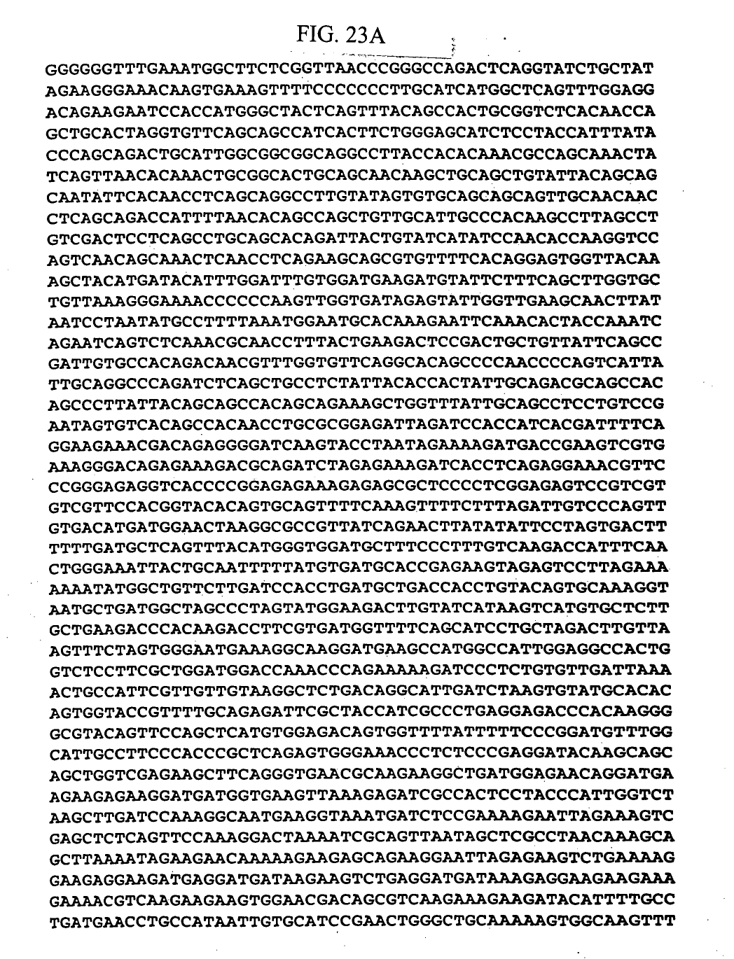

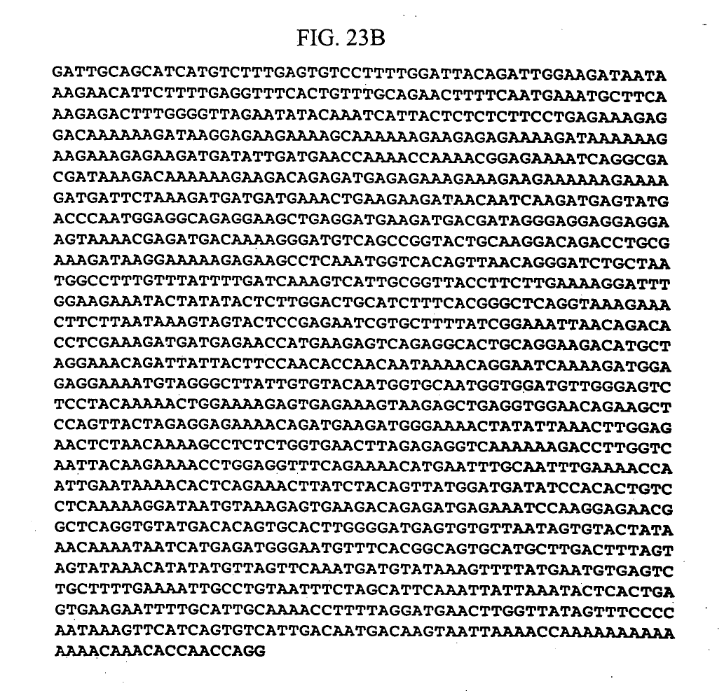

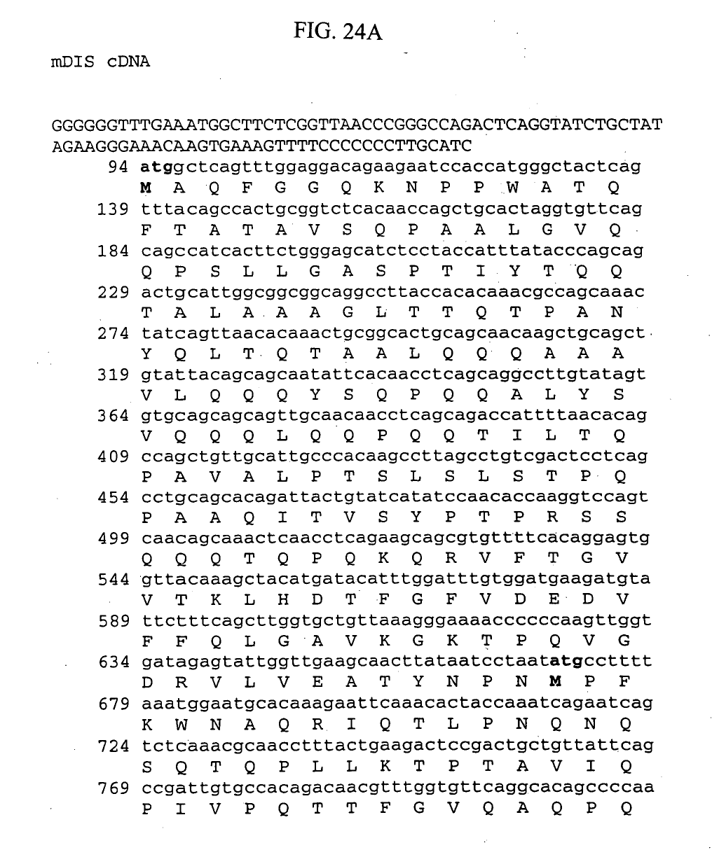

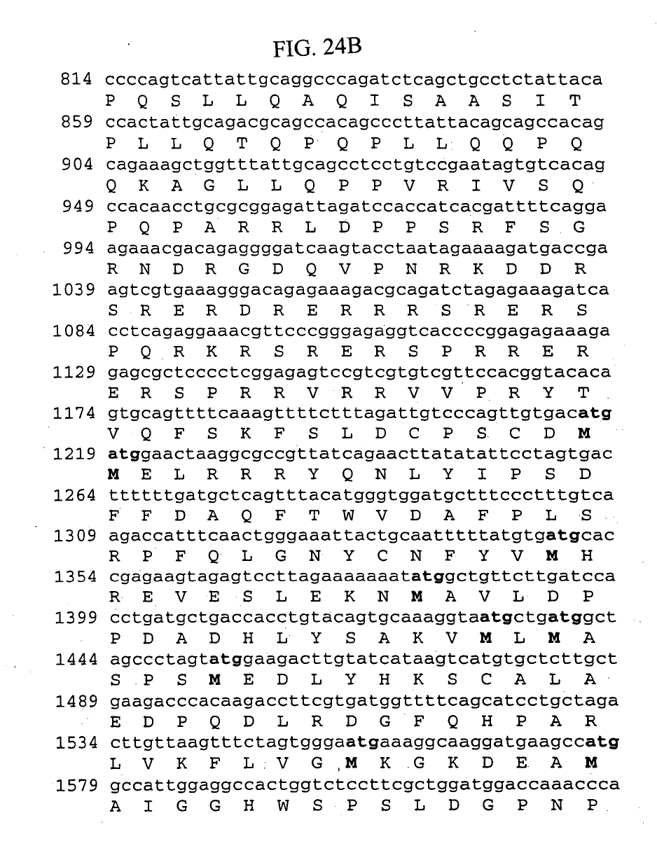

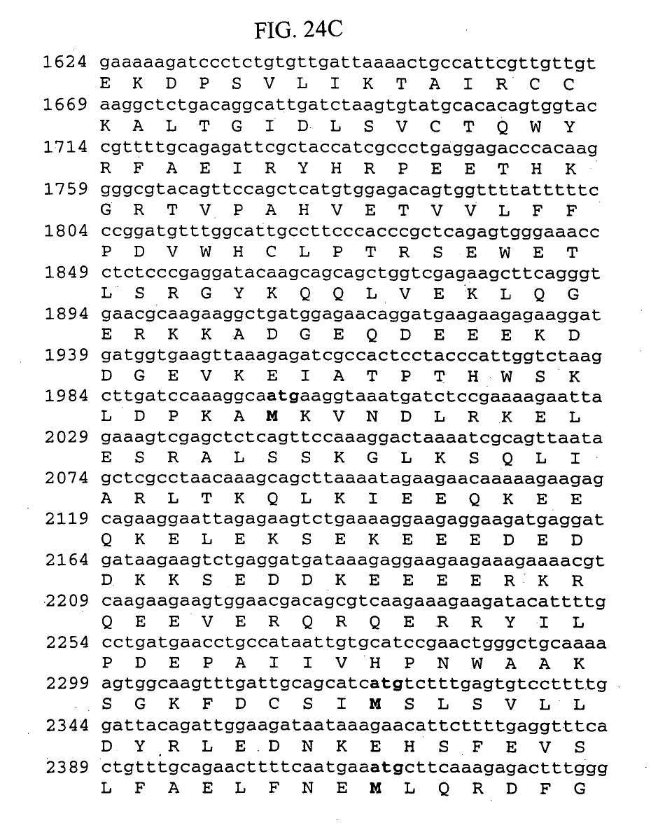

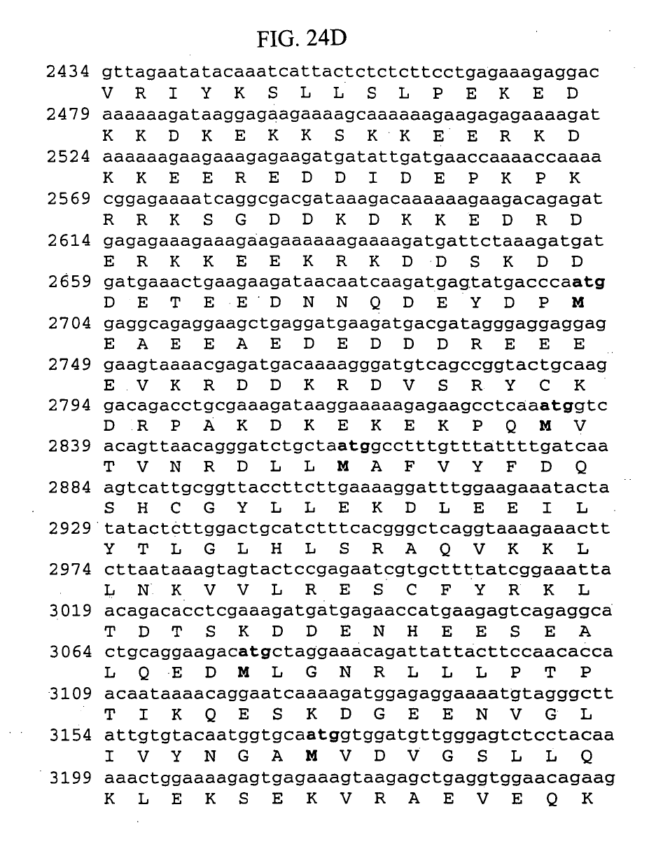

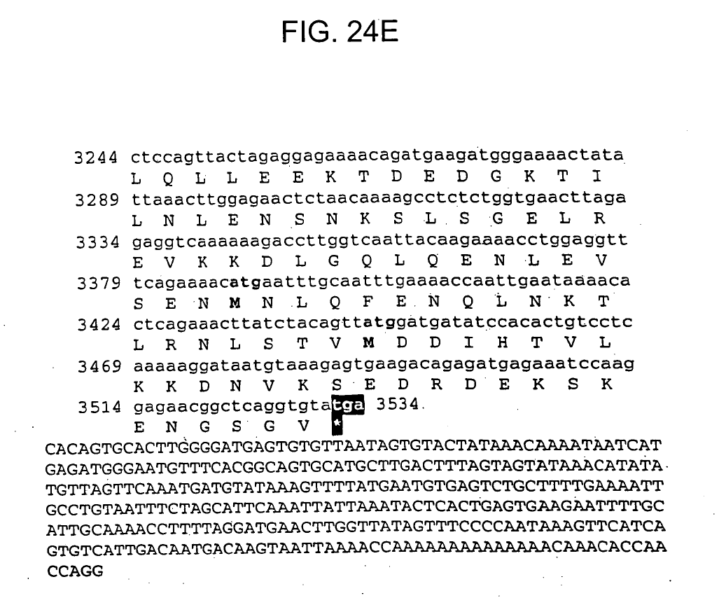

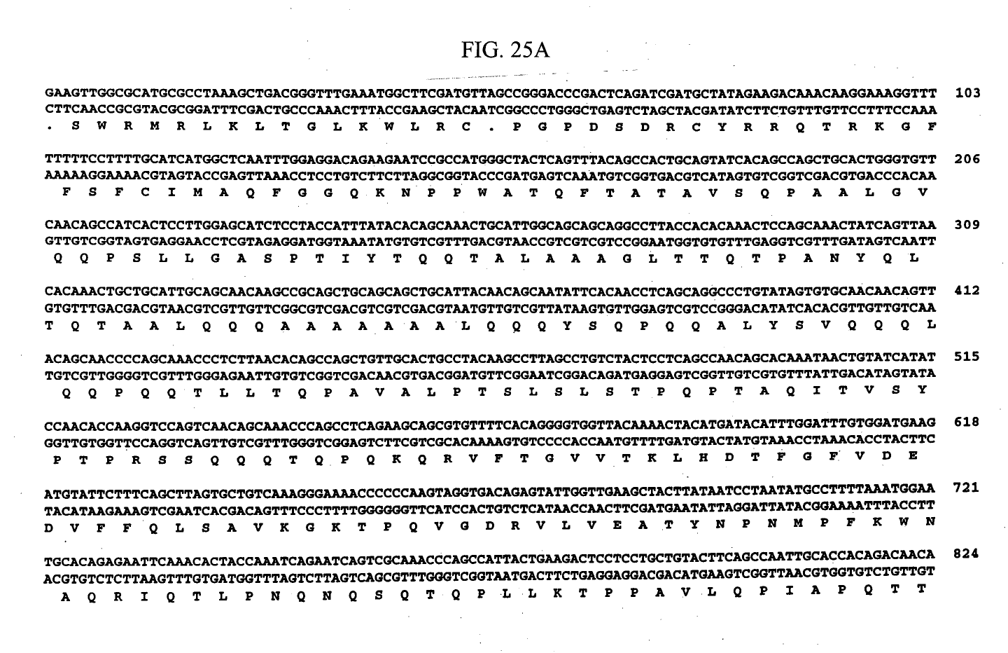

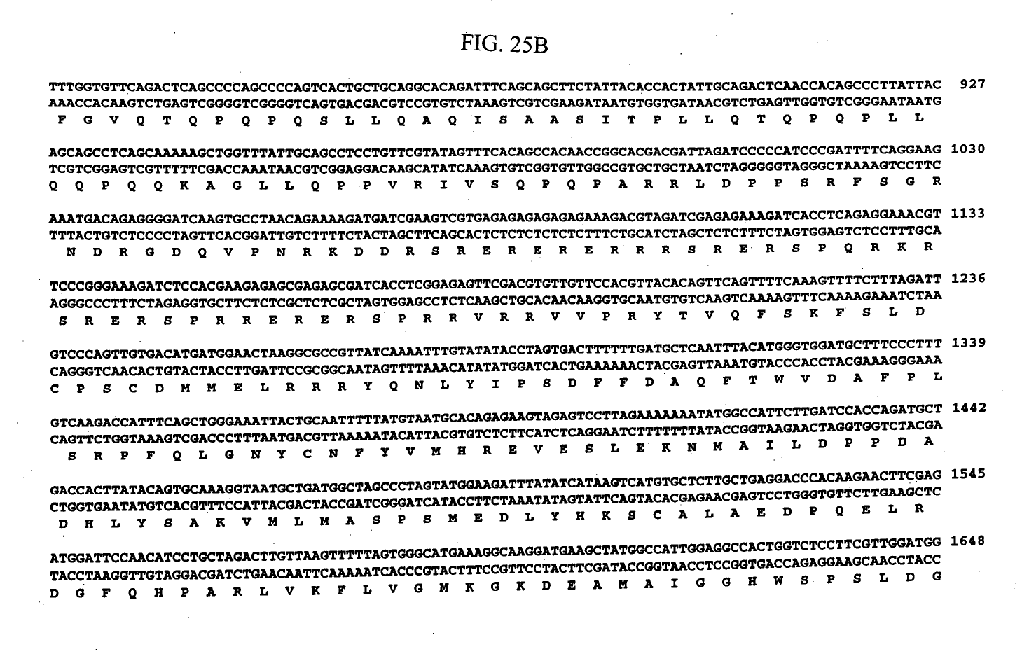

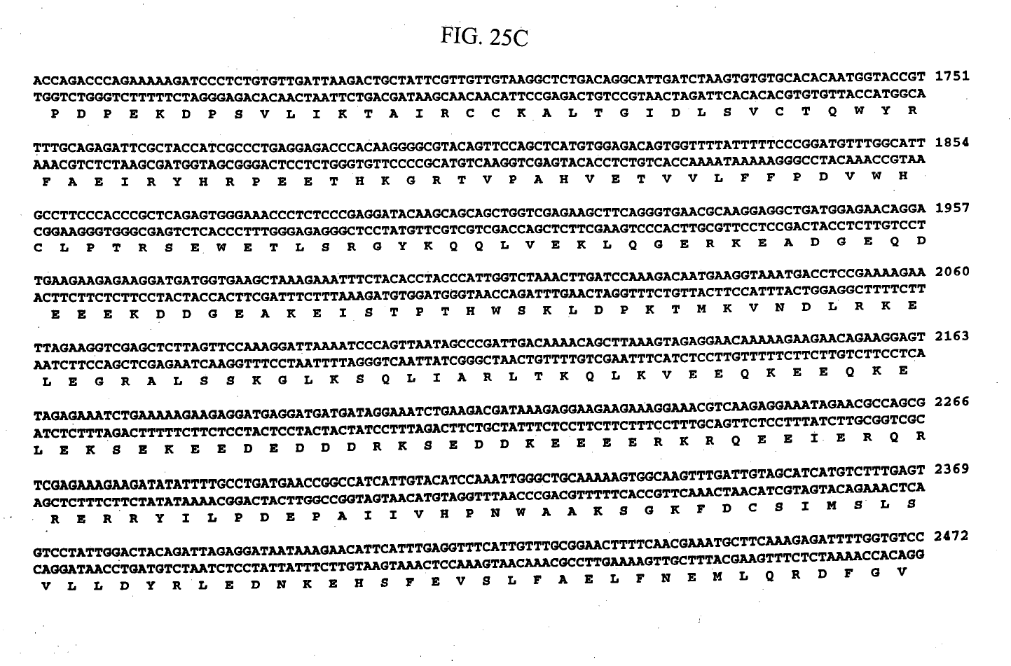

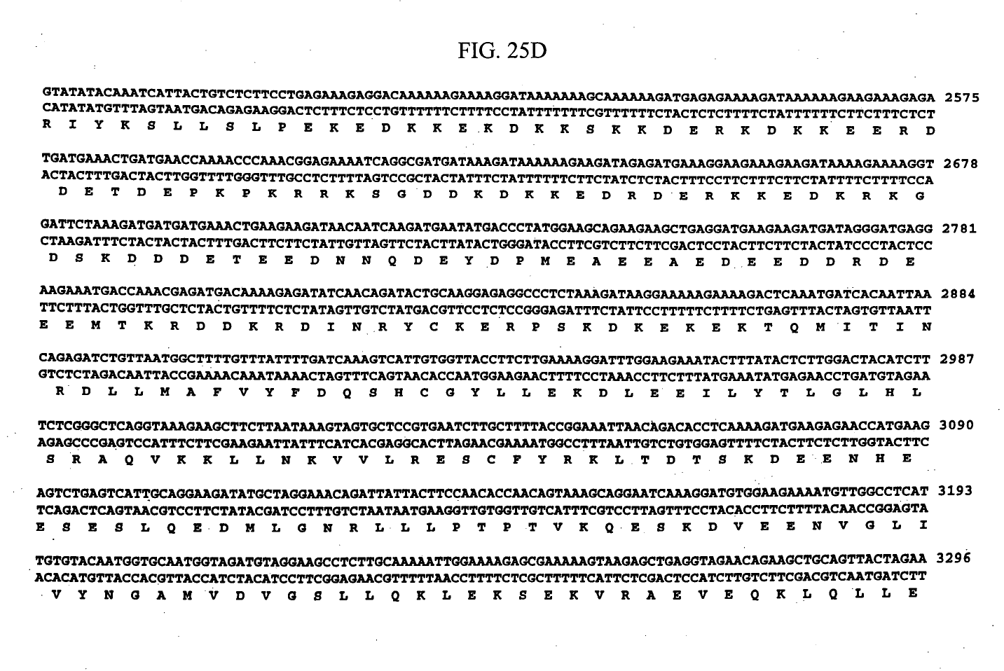

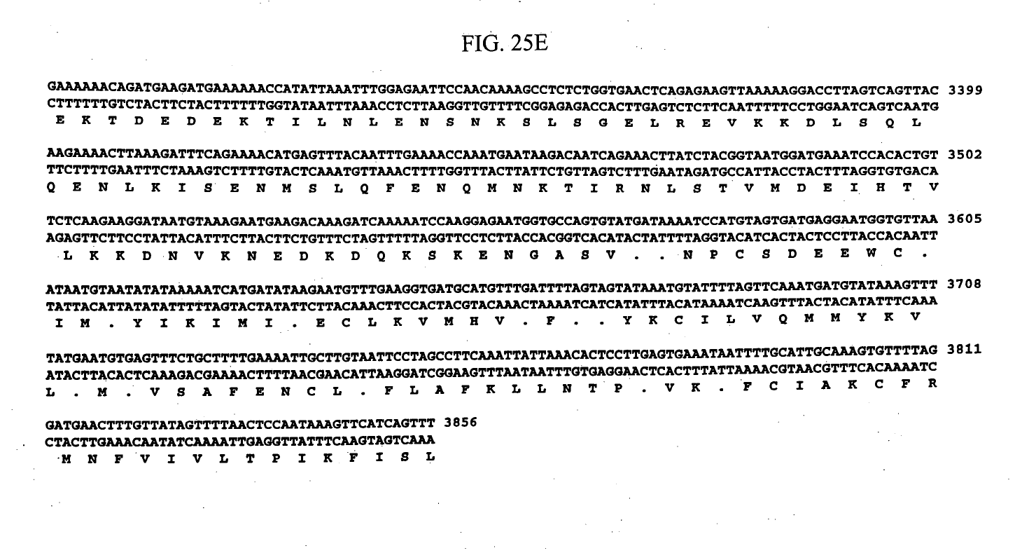

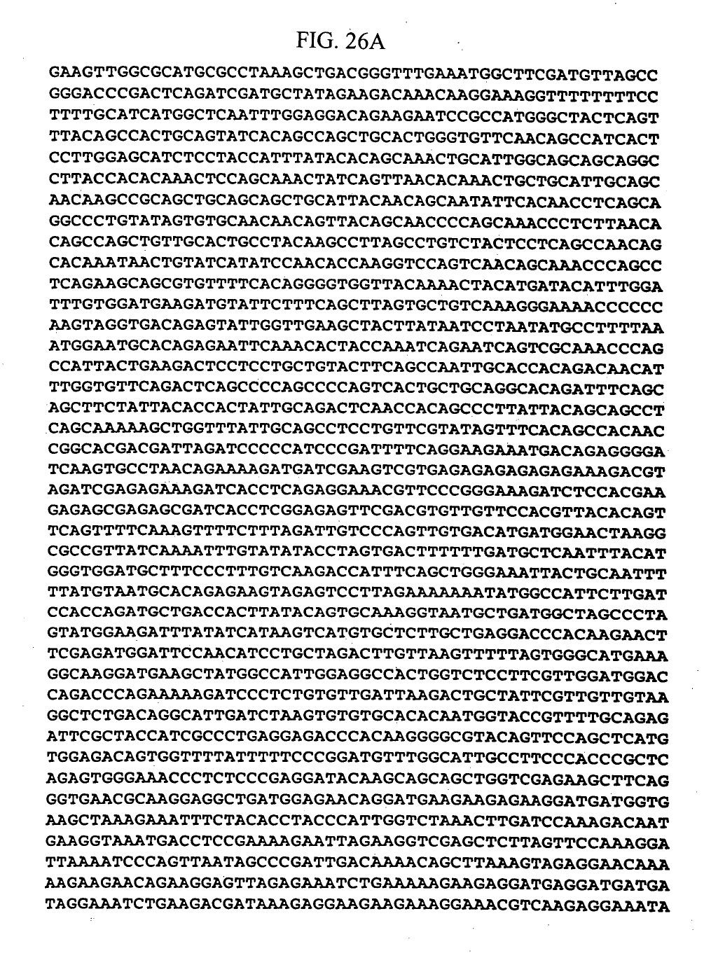

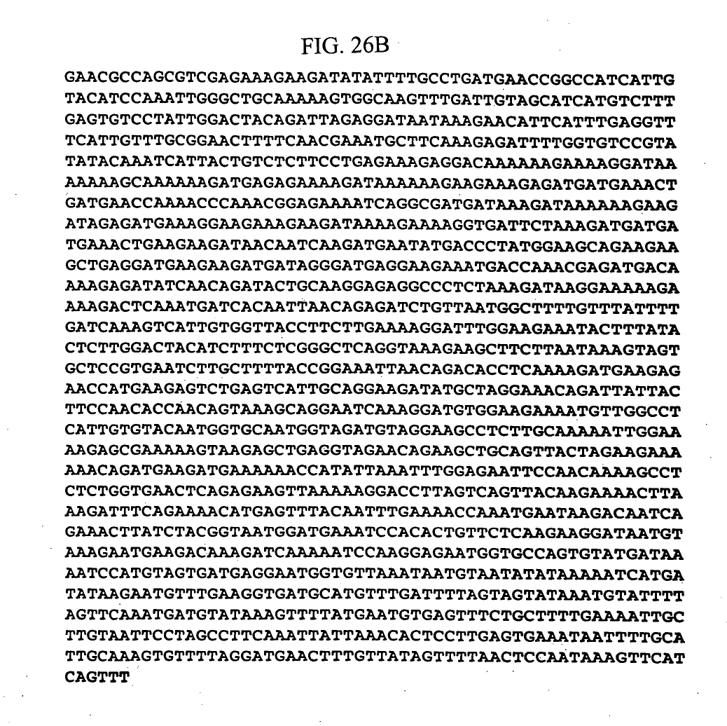

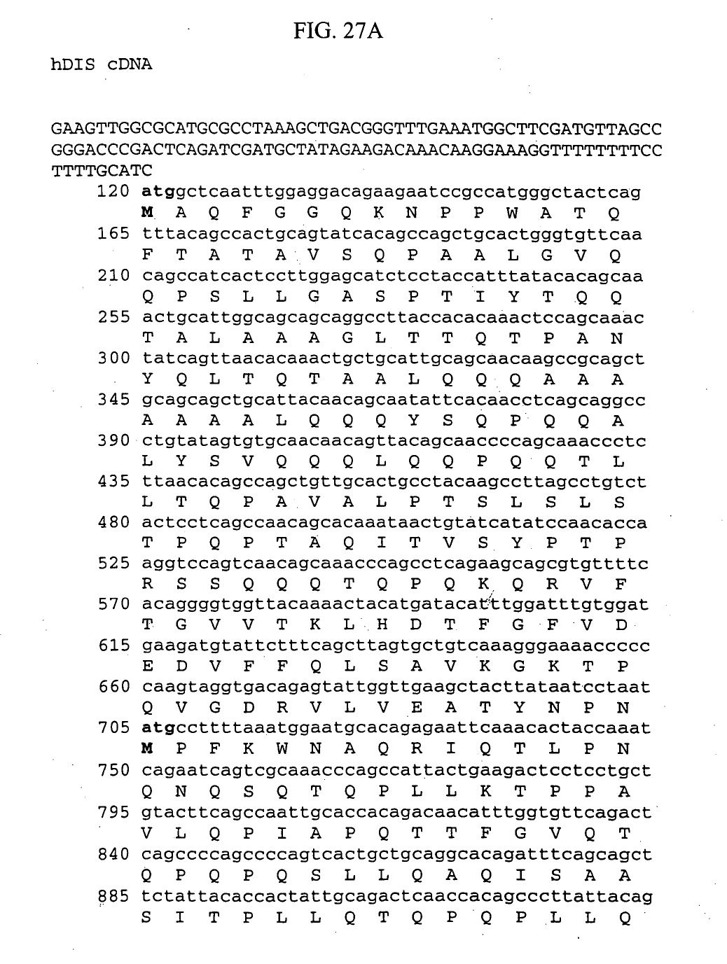

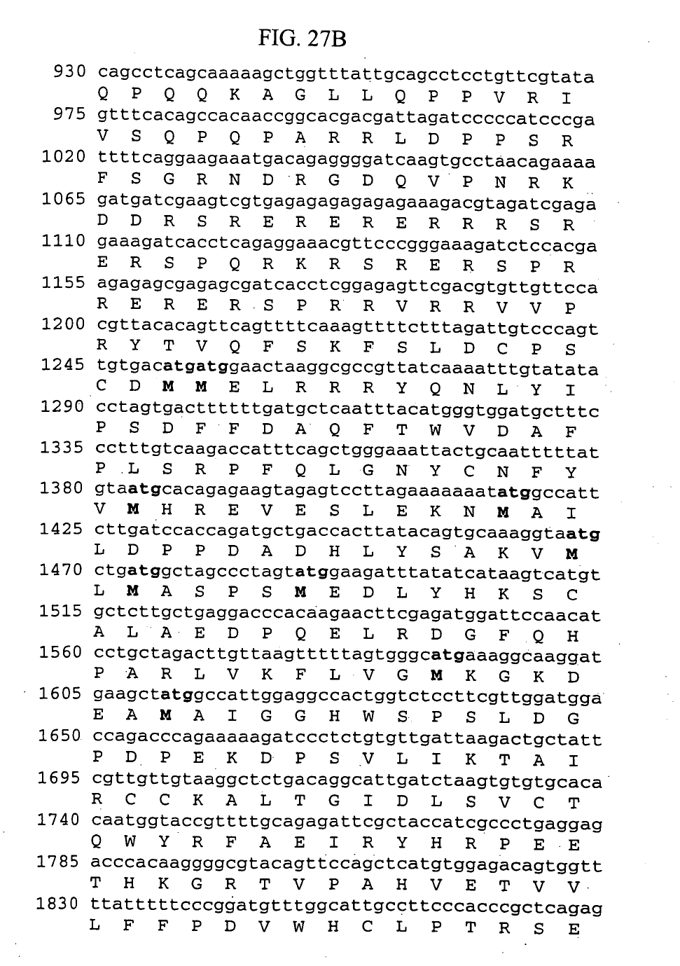

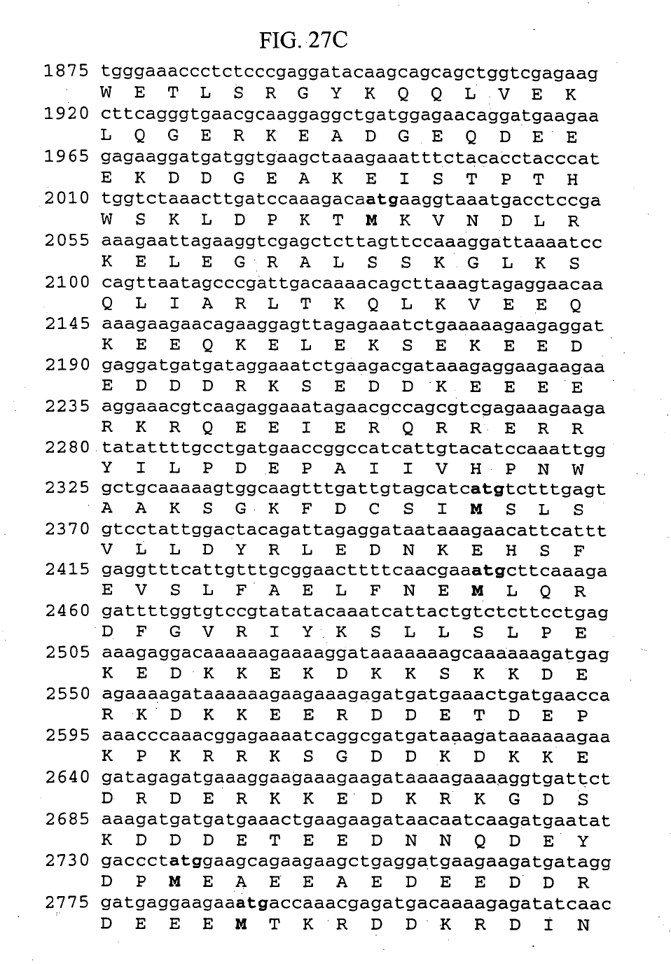

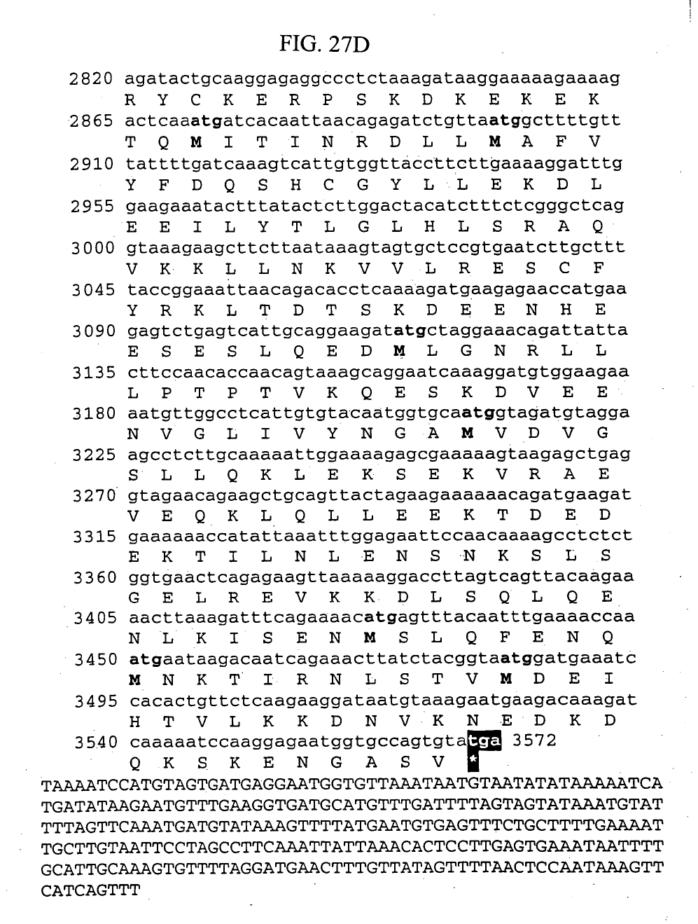

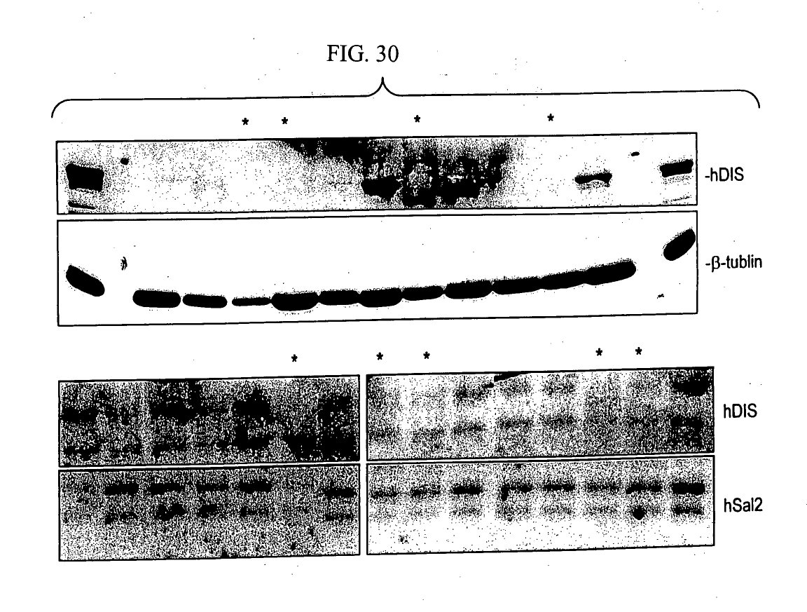

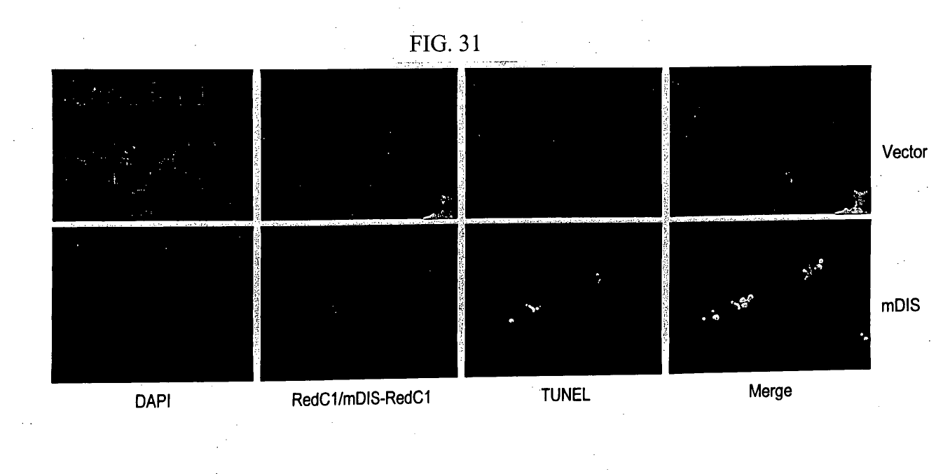

DIS, on the other hand, is a novel protein. We cloned both the mouse and the human cDNAs encoding this protein, the sequences of which are provided in FIG. 22-27. DIS contains an SAP domain, which is a putative DNA-binding motif involved in chromosomal organization (Aravind et al., Trends Biochem. Sci. 25:112-114, 2000). The human DIS gene is located at chromosomal position 10Q22.1 where a loss of heterozygosity has been reported in endometrial and prostate adenocarcinomas. We show that a loss of heterozygosity for DIS exists in human ovarian tumors (FIG. 30, panel A) and that DIS is not expressed in ovarian tumors (FIG. 30, panel B). A loss of heterozygosity may be determined by standard methods in the art, for example, by using Southern blots, by sequencing, or by PCR analysis (See, for example, Debelenko et al., Hum. Mol. Genet. 6:2285-2290, 1997; and Emmert-Buck et al., Cancer Res. 55:2959-2962, 1995). In addition, we showed that DIS efficiently induces apoptosis in cultured cells. We transfected NIH 3T3 cells with DIS fused with Red (pREDC1, Clontech), and observed that the expression of DIS results in DNA condensation and in TUNEL positive cells (FIG. 31). These two markers indicate that cells are undergoing apoptosis.

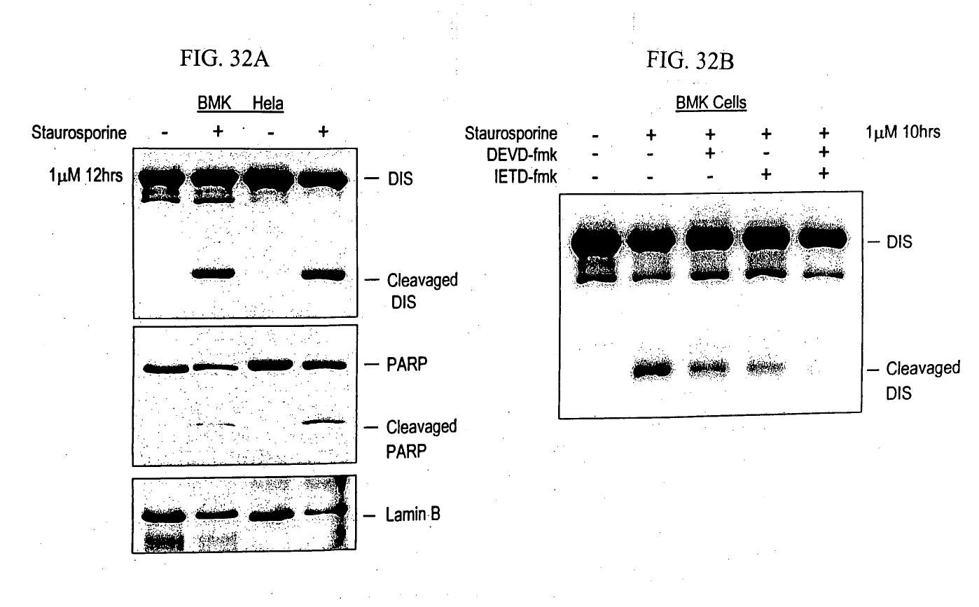

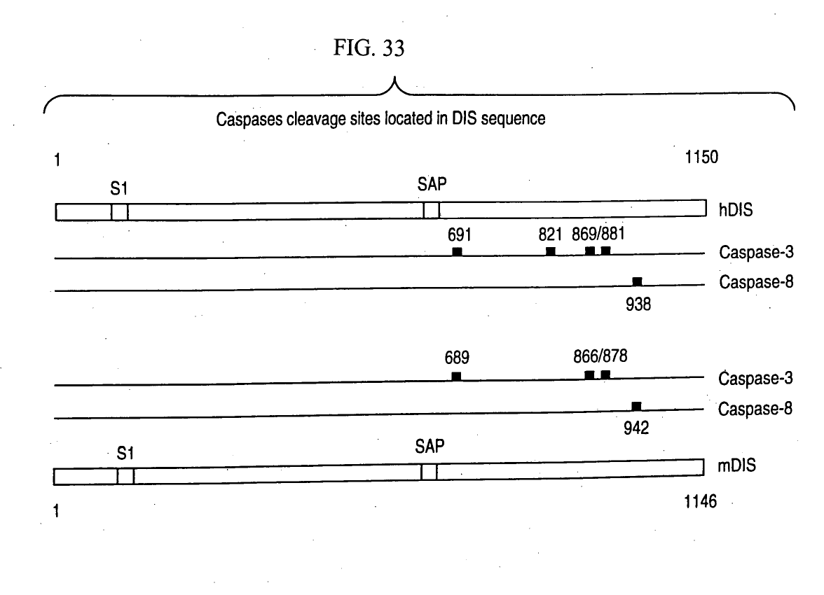

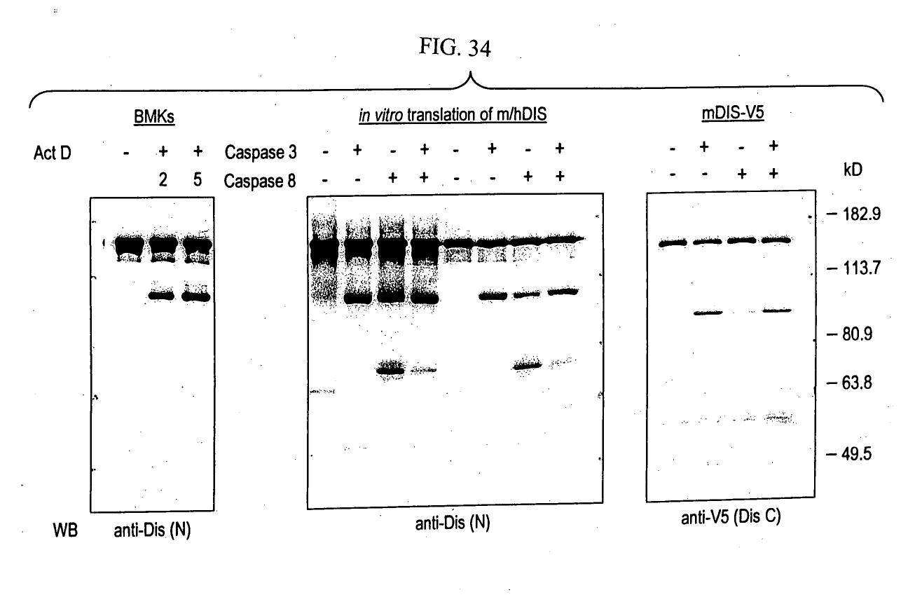

Furthermore, when we induced apoptosis in BMK and HeLa cells with staurosporine, we observed that DIS was degraded and that PARP and LaminB were cleaved (FIG. 32, panel A). Both PARP and LaminB are cleaved by caspases during apoptosis. We also observed that caspase-3 and caspase-8 inhibitors inhibited cleavage of DIS (FIG. 32, panel B). In view of these results, we analyzed the structure of human and mouse DIS and identified a number of caspase-3 and caspase-8 cleavage sites (FIG. 33). In vitro caspase cleavage experiments showed that DIS is sensitive to caspase-3 and that the first caspase-3 site (at amino acid 691 in human DIS (SEQ ID NO:4) and amino acid 689 in murine DIS (SEQ ID NO:2)) is used for cleavage (FIG. 34). Consequently, DIS is likely to function in regulating apoptosis and may be used in methods to diagnose and treat proliferative disorders.

Characterization of TAZ

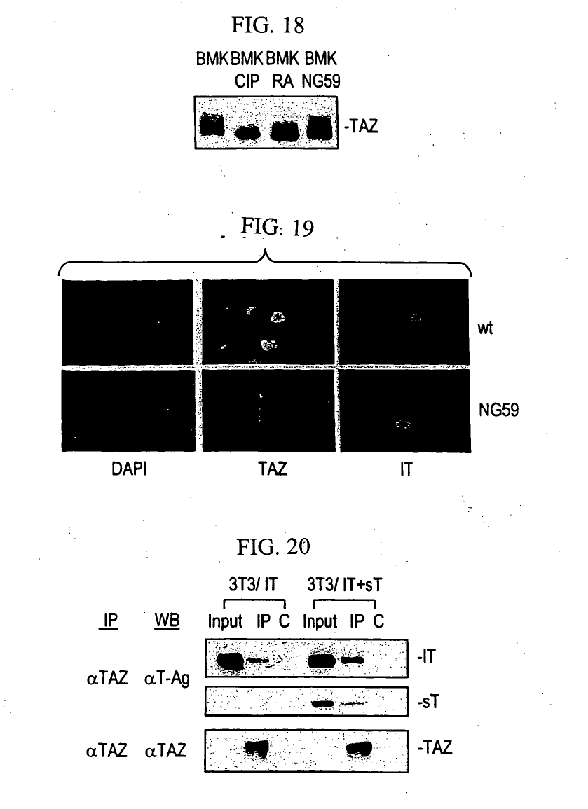

As is noted above, Taz also interacts with PP2A, a phosphatase. Accordingly, we looked at the phosphorylation state of murine Taz and discovered that murine Taz exists in a multiply phosphorylated state in uninfected BMK cells. Taz undergoes dephosphorylation when BMK cells are infected with wild-type polyoma virus, but not when BMK cells are infected with a mutant (NG 59) polyoma virus defective for the middle T antigen. As a control for these experiments, we determined the unphosphorylated state of Taz by adding calf intestinal phosphatase (CIP) to a BMK extract prior to Western blotting (FIG. 18).

Furthermore, we analyzed the intracellular localization of Taz in BMK cells and in these cells infected with either wild-type or NG 59 mutant polyoma virus. The nuclei of the cells were visualized by staining the DNA with DAPI and we used antibodies against Taz and the large T antigen to visualize these proteins. As is seen in FIG. 19, nuclear staining specific for Taz is enhanced in response to BMK cells being infected with wild-type polyoma virus, but not in response to an infection with NG 59 mutant polyoma virus. Additional immunoprecipitation experiments showed that the small T antigen increases binding of Taz to the large T antigen (FIG. 20). These results, in combination with those discussed above, indicate that nuclear import of Taz occurs after binding to the small T antigen and after undergoing dephosphorylation.

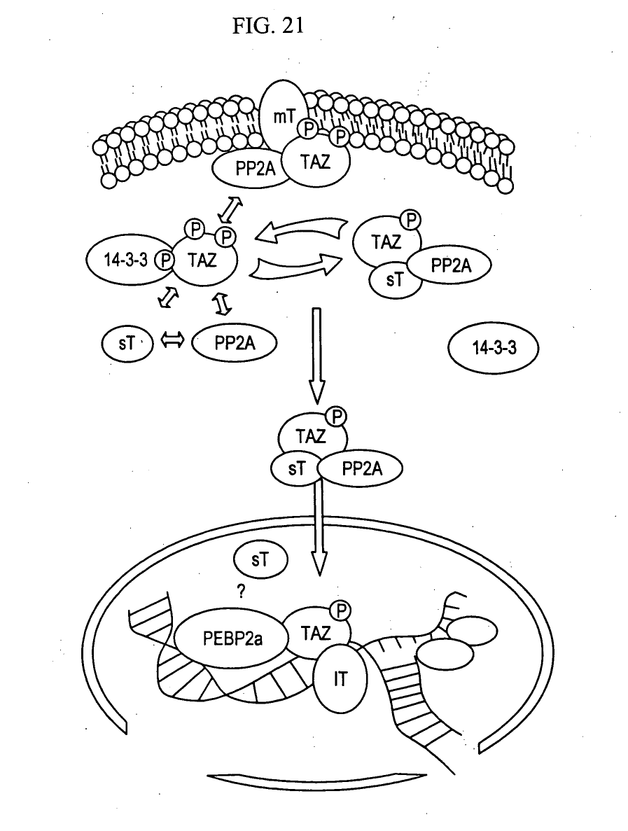

One model of how Taz may function during a polyoma virus infection is shown in FIG. 21. In this non-limiting example Taz may exist in both the nucleus and the cytoplasm. In the cytoplasm, phosphorylated Taz may bind to 14-3-3. After polyoma virus infection, Taz may interact with the small and middle T antigens. These T antigens, in turn, may recruit PP2A to the complex and Taz may undergo dephosphorylation and may dissociate from 14-3-3. Once dephosphorylated, Taz may enter the nucleus where it may bind to the large T antigen (and possibly PEBP2α), and may regulate replication and transcription.

Further support for a role for Taz in regulating transcription comes from data showing that Taz binds to the proline-rich regions of Runx1 (PEBP2α) transcription activator (Kanai et al., EMBO J. 19:6778-6791, 2000). The proline-rich domain that is involved in the interaction between Taz and Runx1 is also found in a number of other transcription factors, including c-Jun, AP-2, C/EBPα, NF-E2, KROX-20, KROX-24, Oct-4, MEF2B, and in the p53 homologue p73. In addition, alterations in members of the Runx family of transcription factors have been found in multiple leukemias and other human cancers (Lo Coco et al., Haematologica 82:364-370, 1997; and Glassman, Clin. Lab. Med. 20:39-48, 2000).

In addition, Taz is closely related to YAP (c-yes-associated protein), a cellular protein identified by its interaction with the SH3 domain of the c-yes proto-oncogene, a member of the Src family of protein tyrosine kinases (Sudol et al., J. Biol. Chem. 270:14733-14741, 1995). Since we show that Taz binds to c-src itself (FIG. 17), and since the interaction between the middle T antigen and c-src is known to play a central role in cell transformation and tumorigenesis by polyoma, Taz is also likely to function in oncogenic pathways.

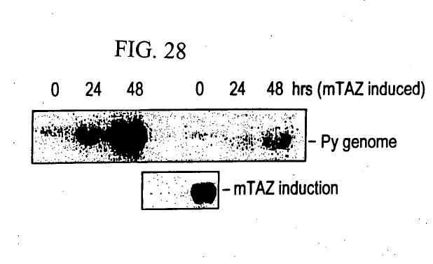

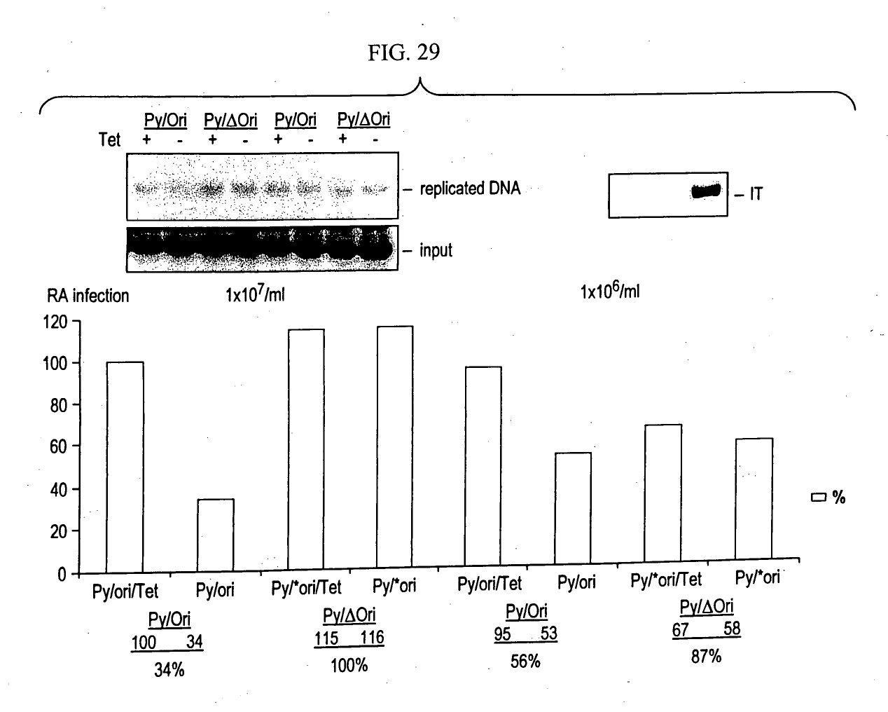

The importance of the interaction between Taz and polyoma T antigens is further demonstrated by our experiments where we showed that over-expression of Taz results in inhibition of polyoma virus replication. For these experiments, we used a 3T3 derived cell line that harbors a tetracycline-regulated mTaz gene. We induced mTaz expression by removing tetracycline 16 hours before infecting these cells, as well as control cells in which mTaz expression was not induced, with wild-type polyoma virus. After infection, we extracted low molecular weight DNA at different time points and performed a Southern blot using ³²P-labeled DNA corresponding to the polyoma origin of replication as a probe (FIG. 28). In addition, our polyoma DNA replication assay results show that TAZ that inhibition of origin replication driven by the virus depends on PEBP2-alpha binding sites located in the origin (FIGS. 29A and 29). PEBP2-alpha is a member of the Runx transcription factor family. Modifications in Runx family members have been implicated in human leukemias and other cancers.

Based on the results of these experiment, we conclude that polyoma virus replication is strongly inhibited in cells over-expressing mTaz. Moreover, in combination with our other observations, these results indicate that an interaction between Taz and polyoma T antigens likely is necessary for a virus to replicate and disseminate and that providing additional Taz, more than can be bound by polyoma T antigens, results in a reduction in the ability of the virus to effectively propagate. Consequently, Taz nucleic acids and amino acids are likely to be desirable anti-viral agents.

In short, in view of the data presented above, including that Taz binds the proto-oncogene product c-src, that Taz binds to multiple transcription factors known to be associated with human cancers including those in the Runx family, e.g., PEBP2α, and that the BMD-13 T-HR mutant, which fails to interact with Taz, also fails to transform cells, Taz is likely to be linked to oncogenic pathways. Accordingly, Taz nucleic and amino acids may be used in a variety of methods to diagnose and treat proliferative disorders.

Diagnosis and Risk Assessment

In addition to helping identify genes that are altered in cancerous cells, target gene profiles can also be used to diagnose and/or stage various proliferative disorders and for diagnosing pre-symptomatic genetic lesions in normal tissues. Accordingly, the methods of the present invention can be used to diagnose cancerous cells in a patient by determining whether the cells of the patient can act as permissive hosts for the growth of a mutant virus, particularly a T-HR mutant (e.g., BMD-13 T-HR mutant virus). As described above, a permissive host for the growth of a mutant virus (e.g., a mutant virus that lacks a functioning transforming protein) has a mutation in a cellular gene (e.g., a Taz, GAP SH3 binding protein, nucleolin, Vesicle-Associated Protein-1, or DIS gene) that is the target for the wild-type viral protein corresponding to the mutant viral protein. This cellular mutation is believed to compensate for the modification of function in a particular gene in the T-HR mutant and contribute to the abnormal or cancerous phenotype of the cell. However, the permissive host may also have a mutation in a cellular gene encoding a protein that interacts with a protein that binds to a viral protein, or in a cellular gene that encodes a component of a signaling pathway that is required for viral transformation. This information then may be used to screen a population as a whole for individuals that are at an increased risk of developing a particular type of proliferative disorder and also may be used to further characterize the cancer cell (e.g., to grade the stage to which the cancer has progressed).

Once a target protein has been identified, tests for the lack of interaction of the cellular protein with the mutant viral protein are used to confirm the specificity of the interaction of the cellular protein with the wild-type (transforming) protein. A lack of interaction indicates that binding of the wild-type viral protein to the cellular protein is specific. Protein interaction can be verified by numerous methods know to those skilled in the art, including, for example, yeast two-hybrid assays, GST-pull down assays, co-immunoprecipitation, and Far-Western analysis. General guidance regarding these techniques can be found in standard laboratory manuals, such as Ausubel et al. (Current Protocols in Molecular Biology, John Wiley & Sons, New York, N.Y., (1994)), and Sambrook et al. (Molecular Cloning: A Laboratory Manual, Cold Spring Harbor Laboratory, N.Y., (1989)). Once an interaction between the wild-type viral protein and the cellular protein is confirmed, the complete gene and gene product can readily be identified by those skilled in the art using, for example, the methods described below.

The present invention recognizes that the T-HR mutant selection procedures identified herein may identify mutant cellular genes, and their encoded protein products, e.g., cellular genes encoding cell cycle proteins, tumor suppressors, proto-oncogenes, transcriptional factors, regulators of apoptosis, etc., that have genetic lesions associated with a particular proliferative disorder. Those skilled in the art will appreciate that many proliferative disorders, such as cancers, correlate with a particular mutation or mutations in the DNA of a patient. By comparing the sequence for a particular gene in both normal and tumor tissue from the same patient, one can determine if the mutation is of somatic or germline origin. This information that may be used to screen a population as a whole for individuals that are at an increased risk of developing a particular type of proliferative disorder.

The present invention therefore provides a method of identifying a genetic lesion in a cell by determining whether a cell can act as a permissive host for the growth of a particular T-HR mutant, such a T-HR mutant virus being capable of growing on a cell having a specific genetic lesion and not being capable of growth on a cell lacking this genetic lesion. This type of information may even be used to further characterize the cancer cell (e.g., to grade the stage to which the cancer has progressed).

In addition, the cellular gene that encodes a protein that is a target for a viral transforming protein may also be analyzed to determine whether there is a genetic lesion in the cellular gene. Such a genetic lesion may be associated with a particular cancer. As noted above, the present inventors describe a genetic lesion that may be associated with ovarian cancer has been identified in a Sal2 gene. Specifically, this genetic lesion, corresponding to the substitution of a Cys for the Ser at position 73 in protein encoded by the mSal2 gene of SEQ ID NO:4, has been identified in DNA from blood samples from patients with ovarian cancer. Probes and primers based on this genetic lesion may be used as markers to detect the Ser73Cys change in samples from other patients.

Once a genetic lesion is identified using the methods of the invention (as is described above), the genetic lesion is analyzed for association with an increased risk of developing a proliferative disorder. In this respect, the present invention provides a method of detecting the presence of a genetic lesion in a human Sal2 gene in a physiological sample, however the method is not limited to the Sal2 gene, but rather can be applied to any gene that is associated with an increased risk for developing a proliferative disorder.

As an example, a BMD-13 T-HR mutant may be used to determine whether there is a genetic lesion in a G3BP, nucleolin, Vesicle Associated Protein 1, or DIS gene. Once identified, probes and primers based on this genetic lesion may be used as markers to detect the particular change in samples from other patients.

A genetic lesion in a candidate gene may be identified in a biological sample obtained from a patient using a variety of methods available to those skilled in the art. Generally, these techniques involve PCR amplification of nucleic acid from the patient sample, followed by identification of the genetic lesion by either altered hybridization, aberrant electrophoretic gel migration, restriction fragment length polymorphism. (RFLP) analysis, binding or cleavage mediated by mismatch binding proteins, or direct nucleic acid sequencing. Any of these techniques may be used to facilitate detection of a genetic lesion in a candidate gene, and each is well known in the art; examples of particular techniques are described, without limitation, in Orita et al. (Proc. Natl. Acad. Sci. USA 86:2766-2770, 1989) and Sheffield et al. (Proc. Natl. Acad. Sci. USA 86:232-236 (1989)). Furthermore, expression of the candidate gene in a biological sample (e.g., a biopsy) may be monitored by standard Northern blot analysis or may be aided by PCR (see, e.g., Ausubel et al., Current Protocols in Molecular Biology, John Wiley & Sons, New York, N.Y. (1995); PCR Technology: Principles and Applications for DNA Amplification, H. A. Ehrlich, Ed., Stockton Press, NY; Yap et al., Nucl. Acids. Res. 19:4294 (1991)). Once a genetic lesion is identified using the methods of the invention (as is described above), the genetic lesion is analyzed for association with an increased risk of developing a proliferative disorder.

Furthermore, antibodies against a protein produced by the gene included in the genetic lesion, for example the Taz, G3BP, Nucleolin, Vesicle Associated Protein 1, or DIS protein, may be used to detect altered expression levels of the protein, including a lack of expression, or a change in its mobility on a gel, indicating a change in structure or size. In addition, antibodies may be used for detecting an alteration in the expression pattern or the sub-cellular localization of the protein. Such antibodies include ones that recognize both the wild-type and mutant protein, as well as ones that are specific for either the wild-type or an altered form of the protein. We showed that a polyclonal rabbit anti-Taz antibody specifically recognizes Taz on Western blots and in cell culture. If desired, monoclonal antibodies may also be prepared using the Taz proteins described above and standard hybridoma technology (see, e.g., Kohler et al., Nature 256:495 (1975); Kohler et al., Eur. J. Immunol. 6:511 (1976); Kohler et al., Eur. J. Immunol. 6:292 (1976); Hammerling et al., In Monoclonal Antibodies and T Cell Hybridomas, Elsevier, New York, N.Y. (1981); Ausubel et al., Current Protocols in Molecular Biology, John Wiley & Sons, New York, N.Y. (2000)). Once produced, monoclonal antibodies are also tested for specific Taz protein recognition by Western blot or immunoprecipitation analysis (by the methods described in, for example, Ausubel et al. (Current Protocols in Molecular Biology, John Wiley & Sons, New York, N.Y. (2000)).

Antibodies used in the methods of the invention may be produced using amino acid sequences that do not reside within highly conserved regions, and that appear likely to be antigenic, as analyzed by criteria such as those provided by the Peptide Structure Program (Genetics Computer Group Sequence Analysis Package, Program Manual for the GCG Package, Version 7, 1991) using the algorithm of Jameson and Wolf (CABIOS 4:181 (1988)). These fragments can be generated by standard techniques, e.g., by the PCR, and cloned into the pGEX expression vector (Ausubel et al., Current Protocols in Molecular Biology, John Wiley & Sons, New York, N.Y. (1995)). GST fusion proteins are expressed in E. coli and purified using a glutathione agarose affinity matrix as described in Ausubel et al. (Current Protocols in Molecular Biology, John Wiley & Sons, New York, N.Y., (1995)).

To generate rabbit polyclonal antibodies, and to minimize the potential for obtaining antisera that is non-specific, or exhibits low-affinity binding, two or three fusions are generated for each protein, and each fusion is injected into at least two rabbits. Antisera are raised by injections in series, preferably including at least three booster injections. These methods for antibody production and characterization are applicable to any other protein that is identified by the methods of the invention.

The antibody may be used in immunoassays to detect or monitor protein expression, e.g., Sal2 protein expression, in a biological sample. A polyclonal or monoclonal antibody (produced as described above) may be used in any standard immunoassay format (e.g., ELISA, Western blot, or RIA) to measure polypeptide levels. These levels may be compared to normal levels. Examples of immunoassays are described, e.g., in Ausubel et al. (Current Protocols in Molecular Biology, John Wiley & Sons, New York, N.Y. (1994)). Immunohistochemical techniques may also be utilized for protein detection. For example, a tissue sample may be obtained from a patient, sectioned, and stained for the presence of Sal2 using an anti-Sal2 antibody and any standard detection system (e.g., one which includes a secondary antibody conjugated to horseradish peroxidase). General guidance regarding such techniques can be found in, e.g., Bancroft and Stevens (Theory and Practice of Histological Techniques, Churchill Livingstone (1982); and Ausubel et al., Current Protocols in Molecular Biology, John Wiley & Sons, New York, N.Y. (1994)).

Use of hSal2 as a Diagnostic Tool

As an example of the utility of this approach, the likelihood that hSal2 functions as a tumor suppressor for ovarian cancer has been explored directly by screening a number of ovarian carcinomas for expression of p150^{sal2} and for mutations in the gene (further described below). Approximately 80% of the tumors examined were negative or showed altered or reduced patterns of expression by Western analysis. Immunolocalization in frozen tissue sections showed strong staining in nuclei of epithelial cells on the surface of the normal ovary. In most instances, tumor cells showed a complete lack of staining. However, when staining was present in otherwise negative tumors, cytoplasmic rather than nuclear staining was seen in some areas.

Further evidence for hSal2 function as a tumor suppressor comes from a limited screen for mutations in hSal2, which uncovered point mutations in four cases. In addition, cytogenetic approaches and sequencing efforts utilizing microsatellite markers have been used to map hSal2 adjacent to, and possibly overlapping with, a chromosomal region associated with loss of homozygosity in ovarian (Bandera et al., Cancer Res. 57:513-515 (1997)) and other cancers, e.g., bladder cancer (Chang et al., Cancer Res. 55:3246-3249 (1995)). Such approaches may continue to be used to map hSal2 more precisely.

The mSal2 gene identified by the present invention may further be used to elucidate the cellular pathways of tumor suppression that regulate key cell cycle events. Alternatively, mSal2 may be used to screen for potential tumors, e.g., lung tumors, brain tumors, stomach tumors, prostate tumors; ovarian tumors, tumors in SCID mice, as well as in knockout or transgenic animals, as discussed in detail below.

Treatment

In addition to providing a method for identifying genes altered in cancer cells and diagnosing patients who carry such mutation, the invention further provides various methods for treating or preventing proliferative disorders. One such method involves killing an abnormally proliferating cell using a tumor host range mutant virus. Thus, T-HR mutants may be used to specifically target and kill cancer cells in an organism. Since these viruses can only propagate in cells that carry a mutation in a cellular gene that the virus would normally have to activate (in the case of proto-oncogene) or inactivate (in the case of a tumor suppressor gene) in order to propagate, this virus would be specific to abnormal cells. Accordingly, T-HR mutants may be used to specifically eliminate cancer cells from a patient. For example, a T-HR mutant (i.e., a polyomavirus carrying an altered large T antigen causing it to be defective in replication and tumor induction) may be used to selectively kill human ovarian cancer cells that carry a genetic lesion in the hSal2 gene, such as the Ser73Cys substitution described above.

As another example, the invention provides a method of killing an abnormally proliferating cell using a BMD-13 T-HR mutant virus. These T-HR mutants can be used to specifically target and destroy cancer cells in an organism. Since these mutant viruses can only propagate in cells that carry a mutation in a cellular gene that the virus would normally have to activate, in the case of proto-oncogene, or inactivate, in the case of a tumor suppressor gene, in order to replicate and disseminate, propagation of such a mutant virus would be specific to abnormal cells. Therefore, T-HR mutants can be used to specifically eliminate cancer cells from a patient. For example, a T-HR mutant (e.g., a polyoma virus carrying an alteration in any T antigen causing it to be defective in replication and tumor induction) may be used to selectively kill human leukemia cells that carry a genetic lesion in a Taz gene.

The therapeutic mutant, such as BMD-13 T-HR, may be administered by any of a variety of routes known to those skilled in the art, such as, for example, intraperitoneal, subcutaneous, parenteral, intravenous, intramuscular, or subdermal injection. However, the T-HR mutant may also be administered as an aerosol, as well as orally, nasally, or topically. Standard concentrations used to administer a BMD-13 T-HR mutant include, for example, 10², 10³, 10⁴, 10⁵, or 10⁶ plaque forming units (pfu)/animal, in a pharmacologically acceptable carrier. Appropriate carriers or diluents, as well as what is essential for the preparation of a pharmaceutical composition are described, e.g., in Remington 's Pharmaceutical Sciences (18^{th} edition), ed. A. Gennaro, 1990, Mack Publishing Company, Easton, Pa., a standard reference book in this field.

Formulations for parenteral administration may, for example, contain excipients, sterile water, or saline. For inhalation, formulations may contain excipients, for example, lactose. Aqueous solutions may be used for administration in the form of nasal drops, or as a gel for topical administration. The exact dosage used will depend on the severity of the condition (e.g., the size of the tumor), or the general health of the patient and the route of administration. The T-HR mutant may be administered once, or it may be repeatedly administered as part of a regular treatment regimen over a period of time.

However, one skilled in the art would realize that any number of genes, including ones involved in cell growth, cell cycle regulation, and apoptosis, may be altered in cancer cells. The methods of the invention are applicable to any alteration in a cancer cell that allows a T-HR mutant to grow. Therefore, any cancer that enables a T-HR mutant to propagate can be treated according to the methods of the invention disclosed herein.

Alternatively, the invention provides methods of halting abnormal proliferation in a cell using a target gene of a tumor virus that normally functions as a tumor suppressor gene. Such target genes, for example, mSal2 or Taz, may be identified using a T-HR according to the methods of the invention. A mSal2 or Taz nucleic acid sequence, or a nucleic acid sequence encoding a protein that interacts with mSal2 or Taz (e.g., a DIS nucleic acid sequence), may be introduced into an abnormally proliferating cell, for example, by using liposome-based transfection techniques, to treat the proliferative disorder (Units 9.1-9.4, Ausubel et al., Current Protocols in Molecular Biology, John Wiley & Sons, New York (1995)). Such DNA constructs may also be introduced into mammalian cells using an adenovirus, or retroviral or vaccinia viral vectors (Units 9.10 and 16.15-16.19, Ausubel et al., Current Protocols in Molecular Biology, John Wiley & Sons, New York (1995)). These standard methods of introducing DNA into cells are applicable to a variety of cell-types.

For example, recombinant adenoviral vectors offer several significant advantages for gene transfer. The viruses can be prepared at extremely high titer, infect non-replicating cells, and confer high-efficiency and high-level transduction of target cells in vivo after directed injection or perfusion. Either directed injection or perfusion would be appropriate for delivery of vectors containing a T-HR target gene in a clinical setting. Moreover, transient expression may be sufficient to remove the abnormally proliferating cells and it may be desirable in view of possible bio-safety or toxicity concerns associated with long-term expression of a T-HR target gene.

In animal models, adenoviral gene transfer has generally been found to mediate high-level expression for approximately one week. The duration of transgene expression may be prolonged, and ectopic expression reduced, by using tissue-specific promoters. Other improvements in the molecular engineering of the adenoviral vector itself have produced more sustained transgene expression and less inflammation. This is seen with so-called “second generation” vectors harboring specific mutations in additional early adenoviral genes and “gutless” vectors in which virtually all the viral genes are deleted utilizing a Cre-Lox strategy (Engelhardt, et al., Proc. Natl. Acad. Sci. USA 91:6196-6200,1994; Kochanek, et al., Proc. Natl. Acad. Sci. USA 93:5731-5736, 1996).

In addition, recombinant adeno-associated viruses (rAAV), derived from non-pathogenic parvoviruses, may be used to express a T-HR target gene as these vectors evoke almost no cellular immune response, and produce transgene expression lasting months in most systems. Incorporation of a tissue-specific promoter is, again, beneficial.

Furthermore, besides adenovirus vectors and rAAVs, other vectors and techniques are known in the art, for example, those described by Wattanapitayakul and Bauer (Biomed. Pharmacother. 54:487-504, 2000), and citations therein.

A vector carrying a T-HR target gene can be delivered to the target organ through in vivo perfusion by injecting the vector into the target organ, e.g., the ovary, or into blood vessels supplying this organ (e.g., for the liver, the portal vein (Tada, et al., Liver Transpl. Surg. 4:78-88, 1998) could be used.

Furthermore, a target gene of a T-HR mutant, for example, mSal2, Taz, GAP SH3 binding protein, nucleolin, Vesicle Associated Protein 1, or DIS, may also be used as an anti-viral agent. In the case of a tumor suppressor gene, a DNA tumor virus needs to inactivate the gene to replicate and disseminate. Accordingly, providing active forms of such genes to a cell, or over-expressing these genes in the cell, would effectively interfere with virus replication and dissemination, and, thereby, prevent the virus from causing or contributing to a proliferative disorder. Alternatively, an anti-sense nucleic acid for a proto-oncogene may be used to inactivate such a gene in a cell and, thereby, prevent a DNA tumor virus from activating the gene, or in the case of an abnormally proliferating cell, to halt abnormal proliferation.

Test Compounds

Compounds that may be tested for an effect on proliferative diseases can be from natural as well as synthetic sources. Those skilled in the field or drug discovery and development will understand that the precise source of test extracts or compounds is not critical to the methods of the invention. Examples of such extracts or compounds include, but are not limited to, plant-, fungal-, prokaryotic-, or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceanographics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

A test compound that modulates the expression of a T-HR target gene, or its encoded protein, may be used to treat proliferative diseases such as leukemias and other types of cancer.

Transgenic and Knockout Animals

The present invention provides transgenic and knockout animals, for example, those that develop ovarian tumors and accurately recapitulate many of the features of human tumors, an important contribution, since animal models of ovarian carcinoma, for example, are currently not available. Without limitation, particularly preferred transgenic or knockout animals are those in which the tumorigenic phenotype is fully penetrant, the rate of progression of the neoplasm is rapid, and/or the lifespan of the transgenic or knock-out animal is not shortened by a knockout- or transgene-related pathology in other organs. Of course, it will be appreciated that these traits are not required.

The generation of transgenic or knockout mice may provide a valuable tool for the investigation of human ovarian cancer by generating a mouse model for studying the disease, based on the description of the human Sal2 gene provided above. Preferably, the hSal2 gene is used to produce the transgenic mice or the mSal2 gene is the target of the knockout. However, other Sal2 genes may also be used to produce transgenic mice provided that they are compatible with the mouse genome and that the protein encoded by this gene is able to carry out the function of the mSal2 protein.

In addition, the knockout organism may be a conditional knockout. For example, FRT sequences may be introduced into the organism so that they flank the gene of interest. Transient or continuous expression of the FLP protein may then be used to induce site-directed recombination, resulting in the excision of the gene of interest. The use of the FLP/FRT system is well established in the art and is described in, for example, U.S. Pat. No. 5,527,695, and in Lyznik et al. (Nucleic Acid Research 24:3784-3789 (1996)).

Conditional knockout organisms may also be produced using the Cre-lox recombination system. Cre is an enzyme that excises DNA between two recognition sites termed loxP. The cre transgene may be under the control of an inducible, developmentally regulated, tissue specific, or cell-type specific promoter. In the presence of Cre, the gene, for example a Sal2 gene, flanked by loxP sites is excised, generating a knockout. This system is described, for example, in Kilby et al. (Trends in Genetics 9:413-421 (1993)).

Furthermore, a transgene, such as a mutant Sal2 gene, may be conditionally expressed (e.g., in a tetracycline sensitive manner). For example, the promoter for the Sal2 gene may contain a sequence that is regulated by tetracycline and expression of the Sal2 gene product ceases when tetracycline is administered to the mouse. In this example, a tetracycline-binding operator, tetO, is regulated by the addition of tetracycline, or an analog thereof, to the organism's water or diet. The tetO may be operably-linked to a coding region, for example a mutant Sal2 gene. The system also may include a tetracycline transactivator (tTA), which contains a DNA binding domain that is capable of binding the tetO as well as a polypeptide capable of repressing transcription from the tetO (e.g., the tetracycline repressor (tetR)), and may be further coupled to a transcriptional activation domain (e.g., VP16). When the tTA binds to the tetO sequences, in the absence of tetracycline, transcription of the target gene is activated. However, binding of tetracycline to the tTA prevents activation. Thus, a gene operably-linked to a tetO is expressed in the absence of tetracycline and is repressed in its presence. The tetracycline regulatable system is well known to those skilled in the art and is described in, for example, WO 94/29442, WO 96/40892, WO 96/01313, and Yamamoto et al. (Cell 101:57-66 (2000).

Particularly preferred is a mouse model for ovarian cancer wherein the nucleic acid encoding a Sal2 gene is expressed in the cells of the ovary of the transgenic mouse such that the transgenic mouse develops ovarian tumors. The mice preferably contain a large T antigen transgene, such as one expressing an appropriate (carboxyl-terminal) fragment of large T antigen under the control of an ovarian specific promoter, or have a knockout of the mSal2 gene. In addition, ovarian cell lines from these mice may be established by methods standard in the art.

Transgenic animals may be made using standard techniques. For example, a gene encoding a cellular proto-oncogene, tumor suppressor gene, or other cellular protein, e.g., a cell cycle regulating protein, may be provided using endogenous control sequences or using constitutive, tissue-specific, or inducible regulatory sequences. Any tissue specific promoter may direct the expression of any Sal2 protein used in the invention, such as ovarian specific promoters, bladder specific promoters, and colon specific promoters. For example, knockout mutations may be engineered in the gene encoding the proto-oncogene or tumor suppressor gene and the mutated gene may be used to replace the wild-type Sal2 gene.

Construction of transgenes can be accomplished using any suitable genetic engineering technique, such as those described in Sambrook et al. (Molecular Cloning: A Laboratory Manual, Cold Spring Harbor Laboratory, N.Y., (1989)). Many techniques of transgene construction and of expression constructs for transfection or transformation in general are known and may be used for the disclosed constructs. Although the use of hSal2 in the transgene constructs is used as an example, any other protein encoded by an oncogene may also be used.

One skilled in the art will appreciate that a promoter is chosen that directs expression of the oncogene in the tissue in which cancer is expected to develop. For example, as noted above, any promoter that regulates expression of hSal2 in ovarian cancer cells can be used in the expression constructs of the present invention. Preferred ovarian promoters include, for example, promoters that are expressed in ovarian epithelial cells, such as, the polyoma virus promoter, the SPARK promoter, and the DOC-2 promoter. One skilled in the art would be aware that the modular nature of transcriptional regulatory elements and the absence of position-dependence of the function of some regulatory elements, such as enhancers, make modifications such as, for example, rearrangements, deletions of some elements or extraneous sequences, and insertion of heterologous (i.e., foreign) elements possible. Numerous techniques are available for dissecting the regulatory elements of genes to determine their location and function. Such information can be used to direct modification of the elements, if desired. It is preferred, however, that an intact region of the transcriptional regulatory elements of a gene is used. Once a suitable transgene construct has been made, any suitable technique for introducing this construct into embryonic cells can be used, an example of such a technique is provided in Example 9.

Animals suitable for transgenic experiments can be obtained from standard commercial sources such as Taconic (Germantown, N.Y.). Many strains are suitable, but Swiss Webster (Taconic) female mice are preferred for embryo retrieval and transfer. B6D2F (Taconic) males can be used for mating and vasectomized Swiss Webster studs can be used to stimulate pseudopregnancy. In addition, vasectomized mice and rats are also publicly available from the above-mentioned suppliers. However, one skilled in the art would also know how to make a transgenic mouse or rat. An example of a protocol that can be used to produce a transgenic animal is provided in Example 9.

Use of Transgenic and Knockout Animals

The disclosed transgenic and knockout animals may be used as research tools to determine genetic and physiological features of a cancer, and for identifying compounds that can affect ovarian and other tumors. Knockout animals also include animals where the normal gene has been inactivated or removed and replaced with a mutant form of this gene, for example, a polymorphic allele. These animals can serve as a model system for assessing the risk of acquiring a proliferative disease that is associated with a particular mutation.

In general, the method of identifying markers associated with a proliferative disorder, such as ovarian tumors, involves comparing the presence, absence, or level of expression of genes, either at the RNA level or at the protein level, in tissue from a transgenic or knockout animal as described above, and in tissue from a matching non-transgenic or knockout animal. Standard techniques for detecting RNA expression, e.g., by Northern blotting, or protein expression, e.g., by Western blotting, are well known in the art. Differences between animals such as the presence, absence, or level of expression of a gene indicate that the expression of the gene is a marker associated with a proliferative disorder, such as ovarian tumors. The molecular markers, once identified, can be used to predict whether patients with carcinoma will have indolent or aggressive disease, and may be mediators of disease progression. Identification of such mediators would be useful since they are possible therapeutic targets. Identification of markers can take several forms.

One method by which molecular markers may be identified is by use of directed screens. Patterns of accumulation of a variety of molecules that may regulate growth can be surveyed using immunohistochemical methods. Screens directed at analyzing expression of specific genes or groups of molecules implicated in pathogenesis can be continued during the life of the transgenic or knockout animal. Expression can be monitored by immunohistochemistry as well as by protein and RNA blotting techniques. Mestastatic foci, once formed, can also be subjected to such comparative surveys.

Alternatively, molecular markers may be identified using genomic screens. For example, tissue can be recovered from young transgenic or knockout animals (e.g., that may have early stage cancer) and older transgenic or knockout animals (e.g., that may have advanced stage cancer), and compared with similar material recovered from age-matched normal littermate controls to catalog genes that are induced or repressed as disease is initiated, and as disease progresses to its final stages. These surveys will generally include cellular populations present in the affected tissue. As another example, ovarian tissue can be recovered from young transgenic or knockout animals (e.g., that may have early stage carcinoma) and older transgenic or knockout animals (e.g., that may have advanced stage carcinoma), and compared with similar material recovered from age-matched normal littermate controls to catalog genes that are induced or repressed as disease is initiated, and as disease progresses to its final stages. These surveys will generally include cellular populations in the ovary.

This analysis can also be extended to include an assessment of the effects of various treatment paradigms (including the use of compounds identified as affecting ovarian tumors in the transgenic or knockout animals) on differential gene expression (DGE). The information derived from the surveys of DGE can ultimately be correlated with disease initiation and progression in the transgenic or knockout animals.

This analysis can also be extended to include an assessment of the effects of various treatment paradigms (including the use of compounds identified as affecting cancers in the transgenic or knockout animals) on differential gene expression (DGE). The information derived from the surveys of DGE can ultimately be correlated with disease initiation and progression in the transgenic or knockout animals.

To assess the effectiveness of a treatment paradigm, a transgene, such as a mutant Taz gene, may be conditionally expressed (e.g., in a tetracycline sensitive manner). For example, the promoter for the Taz gene may contain a sequence that is regulated by tetracycline and expression of the Taz gene product ceases when tetracycline is administered to the mouse. In this example, a tetracycline-binding operator, tetO, is regulated by the addition of tetracycline, or an analog thereof, to the organism's water or diet. The tetO may be operably-linked to a coding region, for example a mutant Taz gene. The system also may include a tetracycline transactivator (tTA), which contains a DNA binding domain that is capable of binding the tetO as well as a polypeptide capable of repressing transcription from the tetO (e.g., the tetracycline repressor (tetR)), and may be further coupled to a transcriptional activation domain (e.g., VP16). When the tTA binds to the tetO sequences, in the absence of tetracycline, transcription of the target gene is activated. However, binding of tetracycline to the tTA prevents activation. Thus, a gene operably-linked to a tetO is expressed in the absence of tetracycline and is repressed in its presence. Alternatively, this system could be modified such that a gene is expressed in the presence of tetracycline and repressed in its absence. Tetracycline regulatable systems are well known to those skilled in the art and are described in, for example, WO 94/29442, WO 96/40892, WO 96/01313, and Yamamoto et al. (Cell 101:57-66 (2000).

In addition, the knockout organism may be a conditional, i.e., somatic knockout. For example, FRT sequences may be introduced into the organism so that they flank the gene of interest. Transient or continuous expression of the FLP protein may then be used to induce site-directed recombination, resulting in the excision of the gene of interest. The use of the FLP/FRT system is well established in the art and is described in, for example, U.S. Pat. No. 5,527,695, and in Lyznik et al. (Nucleic Acid Research 24:3784-3789 (1996)).

Conditional, i.e., somatic knockout organisms may also be produced using the Cre-lox recombination system. Cre is an enzyme that excises DNA between two recognition sites termed loxP. The cre transgene may be under the control of an inducible, developmentally regulated, tissue specific, or cell-type specific promoter. In the presence of Cre, the gene, for example a Taz gene, flanked by loxP sites is excised, generating a knockout. This system is described, for example, in Kilby et al. (Trends in Genetics 9:413-421 (1993)).

Particularly desirable is a mouse model for leukemia wherein the nucleic acid having an alteration in a Taz, GAP SH3 binding protein, nucleolin, Vesicle Associated Protein 1, or DIS gene, for example, an altered human Taz gene, is expressed in the blood cells of the transgenic mouse such that the transgenic mouse develops leukemia. The mice may also contain a T antigen transgene, such as one expressing an appropriate (e.g., N-terminally truncated) fragment of a T antigen under the control of a tissue specific promoter, or have a knockout of the murine Taz, GAP SH3 binding protein, nucleolin, Vesicle Associated Protein 1, or DIS gene. In addition, cell lines from these mice may be established by methods standard in the art.

Construction of transgenes can be accomplished using any suitable genetic engineering technique, such as those described in Sambrook et al. (Molecular Cloning: A Laboratory Manual, Cold Spring Harbor Laboratory, N.Y., (1989)). Many techniques of transgene construction and of expression constructs for transfection or transformation in general are known and may be used for the disclosed constructs. Although the use of an altered hTaz gene in the transgene constructs is used as an example, a wild-type or altered GAP SH3 binding protein, nucleolin, Vesicle Associated Protein 1, or DIS gene, or any protein encoded by an oncogene, or by an inactive tumor suppressor gene, may also be used.

One skilled in the art will appreciate that a promoter is chosen that directs expression of the chosen gene in the tissue in which cancer is expected to develop. For example, as noted above, any promoter that promotes expression of Taz in blood cells can be used in the expression constructs of the present invention. One skilled in the art would be aware that the modular nature of transcriptional regulatory elements and the absence of position-dependence of the function of some regulatory elements, such as enhancers, make modifications such as, for example, rearrangements, deletions of some elements or extraneous sequences, and insertion of heterologous elements possible. Numerous techniques are available for dissecting the regulatory elements of genes to determine their location and function. Such information can be used to direct modification of the elements, if desired. It is desirable, however, that an intact region of the transcriptional regulatory elements of a gene is used. Once a suitable transgene construct has been made, any suitable technique for introducing this construct into embryonic cells can be used.

Animals suitable for transgenic experiments can be obtained from standard commercial sources such as Taconic (Germantown, N.Y.). Many strains are suitable, but Swiss Webster (Taconic) female mice are desirable for embryo retrieval and transfer. B6D2F (Taconic) males can be used for mating and vasectomized Swiss Webster studs can be used to stimulate pseudopregnancy. Vasectomized mice and rats are publicly available from the above-mentioned suppliers. However, one skilled in the art would also know how to make a transgenic mouse or rat. An example of a protocol that can be used to produce a transgenic animal is provided below.

Production of Transgenic Mice and Rats

The following is but one desirable means of producing transgenic mice. This general protocol may be modified by those skilled in the art.

Female mice six weeks of age are induced to superovulate with a 5 IU injection (0.1 cc, IP) of pregnant mare serum gonadotropin (PMSG; Sigma) followed 48 hours later by a 5 IU injection (0.1 cc, IP) of human chorionic gonadotropin (hCG, Sigma). Females are placed together with males immediately after hCG injection. Twenty-one hours after hCG injection, the mated females are sacrificed by CO₂ asphyxiation or cervical dislocation and embryos are recovered from excised oviducts and placed in Dulbecco's phosphate buffered saline with 0.5% bovine serum albumin (BSA, Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with humidified atmosphere at 5% CO₂, 95% air until the time of injection. Embryos can be implanted at the two-cell stage.

Randomly cycling adult female mice are paired with vasectomized males. Swiss Webster or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS (Dulbecco's phosphate buffered saline) and in the tip of a transfer pipet (about 10 to 12 embryos). The pipet tip is inserted into the infundibulum and the embryos are transferred. After the transferring the embryos, the incision is closed by two sutures.

A desirable procedure for generating transgenic rats is similar to that described above for mice (Hammer et al., Cell 63:1099-112 (1990). For example, thirty-day old female rats are given a subcutaneous injection of 20 IU of PMSG (0.1 cc) and 48 hours later each female placed with a proven, fertile male. At the same time, 40-80 day old females are placed in cages with vasectomized males. These will provide the foster mothers for embryo transfer. The next morning females are checked for vaginal plugs. Females who have mated with vasectomized males are held aside until the time of transfer. Donor females that have mated are sacrificed (CO₂ asphyxiation) and their oviducts removed, placed in DPBA (Dulbecco's phosphate buffered saline) with 0.5% BSA and the embryos collected. Cumulus cells surrounding the embryos are removed with hyaluronidase (1 mg/ml). The embryos are then washed and placed in EBSs (Earle's balanced salt solution) containing 0.5% BSA in a 37.5° C. incubator until the time of microinjection.

Once the embryos are injected, the live embryos are moved to DPBS for transfer into foster mothers. The foster mothers are anesthetized with ketamine (40 mg/kg, IP) and xulazine (5 mg/kg, IP). A dorsal midline incision is made through the skin and the ovary and oviduct are exposed by an incision through the muscle layer directly over the ovary. The ovarian bursa is torn, the embryos are picked up into the transfer pipet, and the tip of the transfer pipet is inserted into the infundibulum. Approximately 10 to 12 embryos are transferred into each rat oviduct through the infundibulum. The incision is then closed with sutures, and the foster mothers are housed singly.

Generation of Knockout Mice

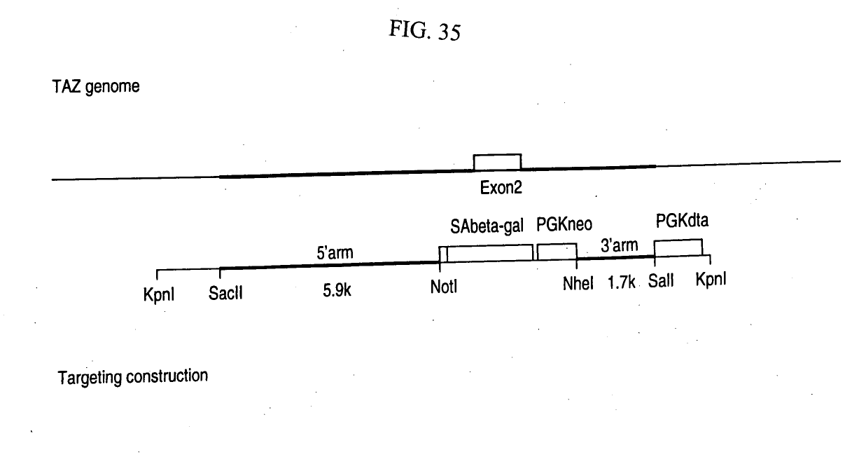

We generated TAZ knock out mice by replacing exon 2 (which encodes amino acids 1-144 of murine DIS; SEQ ID NO:2) of the mouse TAZ gene with the pSAbeta-galpGKneopGKdta positive-negative selection vector (FIG. 35). The TAZ knock out construct was transfected into an Embryonic Stem (ES) cell line and two positive ES clones were obtained and confirmed by PCR and by Southern blot. A Southern blot for the neo gene also confirmed that only exon 2 of TAZ was replaced. We performed microinjections with these ES clones and obtained chimeric mice. Nine F1 TAZ^{+/-} mice were obtained from different chimeric mice and these mice were mated to each other to generate TAZ^{-/-} knockout mice.

In addition to the particular method described above, the following is another example for the generation of a knockout mouse and the protocol may be readily adapted or modified by those skilled in the art.

ES cells, for example, 10⁷ ABI cells, may be electroporated with 25 μg targeting construct in 0.9 ml PBS using a Bio-Rad Gene Pulser (500 μF, 230 V). The cells may then be plated on one or two 10-cm plates containing a monolayer of irradiated STO feeder cells. Twenty-four hours later, they may be subjected to G418 selection (350 μg/ml, Gibco) for 9 days. Resistant clones may then be analyzed by Southern blotting after Hind III digestion, using a probe specific to the targeting construct. Positive clones are expanded and injected into C57BL/6 blastocysts. Male chimeras may be back-crossed to C57BL/6 females. Heterozygotes may be identified by Southern blotting and intercrossed to generate homozygotes.

The targeting construct may result in the disruption of the gene of interest, e.g., by insertion of a heterologous sequence containing stop codons, or the construct may be used to replace the wild-type gene with a mutant form of the same gene, e.g., a “knock-in.” Furthermore, the targeting construct may contain a sequence that allows for conditional expression of the gene of interest. For example, a sequence may be inserted into the gene of interest that results in the protein not being expressed in the presence of tetracycline. Such conditional expression of a gene is described in, for example, Yamamoto et al. (Cell 101:57-66 (2000)).

The following examples are meant to illustrate the invention and should not be construed as limiting.

### EXAMPLES

### Example 1

### Isolation of TMD-25 Using a ‘Tumor Host Range’ Selection

A procedure for isolating ‘tumor host range’ mutants (e.g., T-HR mutants) and identifying cellular targets is outlined below.

Identification of a Host Factor that Interacts with T Antigens
1) Select Host Range Mutants
2) Identify Host Range Mutations
3) Identify Host Range Target and Validation
4) Biological Properties:
   (i) Viral DNA Replication
   (ii) Transformation
   (iii) Tumorigenicity

Permissive hosts were chosen based on a screen of mouse cell lines derived from non-polyoma-induced tumors or transformed cells using the following criteria: (i) susceptibility to lytic infection by wild-type polyoma virus, and (ii) ability to be used in standard plaque assays.

Among a number of qualifying cell lines, two were chosen: A6241, derived from a spontaneous mammary tumor in a C57BR mouse, and TCMK-1, a SV40-transformed baby mouse kidney cell line. Primary baby mouse kidney epithelial cells (BMK) were used throughout as the non-permissive host.

Randomly mutagenized virus was prepared by passage of a plasmid containing wild-type polyoma viral DNA through the error prone Mut D strain of E. coli, followed by excision of the viral genome and transfection into TCMK-1 cells. After several cycles of virus growth in the same cells, individual plaques were isolated using TCMK-1 cells. An aliquot of virus in each plaque suspension was inoculated into BMK cell cultures. Virus from plaques that induced no cytopathic effect (CPE) on BMK cells after 10-14 days was amplified using TCMK-1 cells. Mutant DNAs were cloned, reconstituted as virus by transfection of permissive cells, and confirmed to retain the desired host range. The frequency of mutants was approximately one in several thousand plaques tested. The T-HR mutant TMD-25 was isolated by this procedure.

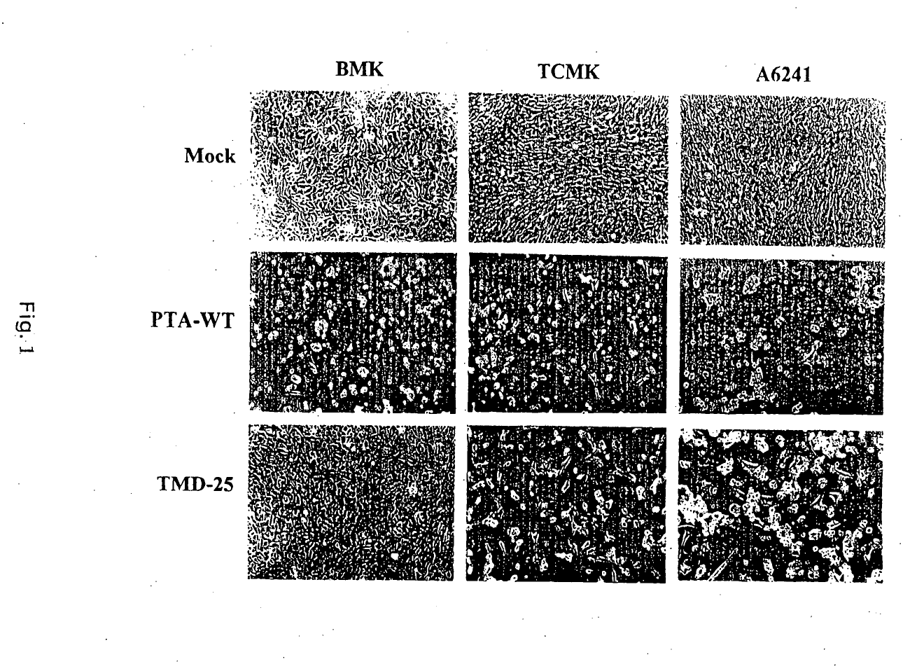

FIG. 1 shows the results of CPE tests comparing wild-type polyoma virus and TMD-25 growth in BMK, TCMK-1, and A6241 cells. Primary baby mouse kidney cells (BMK), SV40 Large T antigen transformed mouse kidney cells (TCMK), and spontaneous mouse mammary tumor cells (A6241) were mock-infected (Mock), or infected with 2-5 pfu of wild-type polyoma virus (PTA) or of T-HR mutant TMD25. The photographs were taken 14 days post infection and show the different cytopathic effects of viral growth.

TMD25 mutants grew poorly, if at all, on primary BMK cells, but could grow on transformed or tumor-derived cells, while wild-type polyoma virus grew well on all three cell-types. Extensive CPE developed in the TCMK-1 and A6241 cultures infected by the TMD25 mutant. Infectious mutant virus was produced in these cultures, although with somewhat slower kinetics and with lower final yields compared to wild-type virus. In contrast, no discernible CPE was noted in mutant-infected BMK cultures, even after extended periods of incubation of up to three weeks. Growth of TMD-25 on the spontaneous tumor line A6241 rules out the possibility that its growth depends strictly on complementation by SV40 large T antigen, which is expressed in TCMK-1.

### Example 2

### Sequencing of TMD-25 and Screening for Targets in Yeast

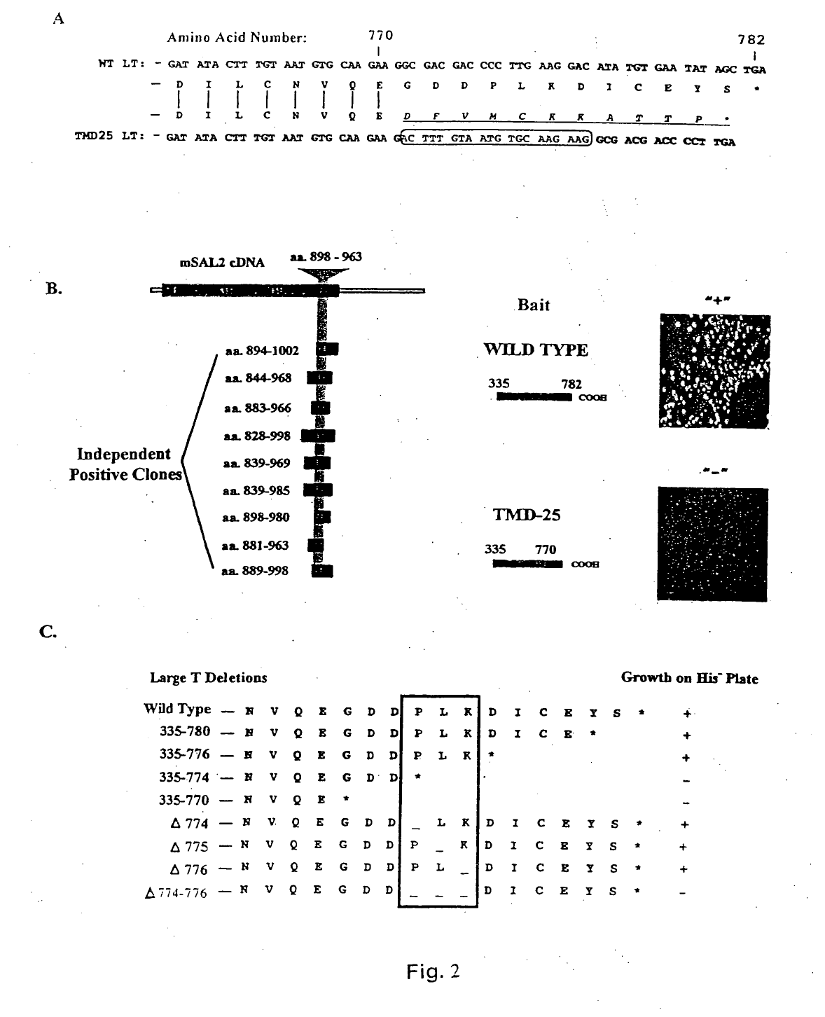

The mutation in TMD-25 responsible for its ‘tumor host range’ was localized to the carboxyl-terminal half of polyoma large T antigen as a result of studies using chimeric viruses constructed by ligating complementary DNA fragments from TMD-25 and wild-type virus. A combination of marker rescue and sequence analysis of this region revealed a twenty base pair duplication (circled) in TMD-25 encompassing the carboxyl-terminus of large T antigen. The resulting frameshift leads to replacement of the last 12 amino acids by 11 foreign residues (underlined) (SEQ ID NOS:9 to 12) (FIG. 2A).

It is possible that the carboxyl-terminal region of large T antigen is involved in binding to some cellular target as an essential step in virus growth and that the mutation in TMD-25 abolishes this interaction. As a first step toward identifying a possible cellular target, a cDNA library constructed from 9.5 to 10.5 day-old mouse embryos was screened in yeast two-hybrid assays, using the carboxyl-terminal portion of normal large T antigen (amino acids 335-782) as bait.

Twenty-two positive clones were analyzed. Nineteen of these clones were represented by nine independent but overlapping cDNA sequences that centered around a sixty-six amino acid region (amino acids 900-965) encompassing a zinc finger pair in the carboxyl-terminal region of the mSal2 protein cDNAs (FIG. 2B, Left Panel, and discussed below). The identified sequences showed strong homology to the human gene hSal2, which is related to spalt in Drosophila.

The positive mSal2 clones did not interact with the carboxyl-terminus of TMD25 large T antigen, as indicated by the growth (+) of yeast colonies on histidine minus plates when using normal polyoma large T antigen as bait, but no growth using TMD25 large T antigen as bait (FIG. 2B, Right Panel), consistent with the notion that the host range defect of TMD-25 is based on its inability to bind this protein. All the His+ yeast colonies were also LacZ positive.

On continuous propagation in permissive cells, the TMD-25 mutant proved to be unstable, giving rise to wild-type virus revertants. To obtain a stable mutant and to further pinpoint the region of large T antigen essential for binding, (SEQ ID NOS:13 to 21), an analysis of the wild-type bait construct was carried out using mSal2 interaction in yeast as an assay (FIG. 2C). Truncation of the last six amino acids had no perceptible effect, but further truncations into the P-L-K sequence at positions 774-776 resulted in a loss of interaction. A deletion of these three amino acids in the context of an otherwise intact large T antigen was sufficient to prevent interaction with mSal2 and to recreate the host range phenotype shown in FIG. 1. The large T antigen deletion mutant 774-776 is hereafter referred to as TMD-25. The original defect of TMD25 is underlined, and the three amino acid region is framed in FIG. 2.

### Example 3

### Validation of mSal2 as a Target of Large T Antigen

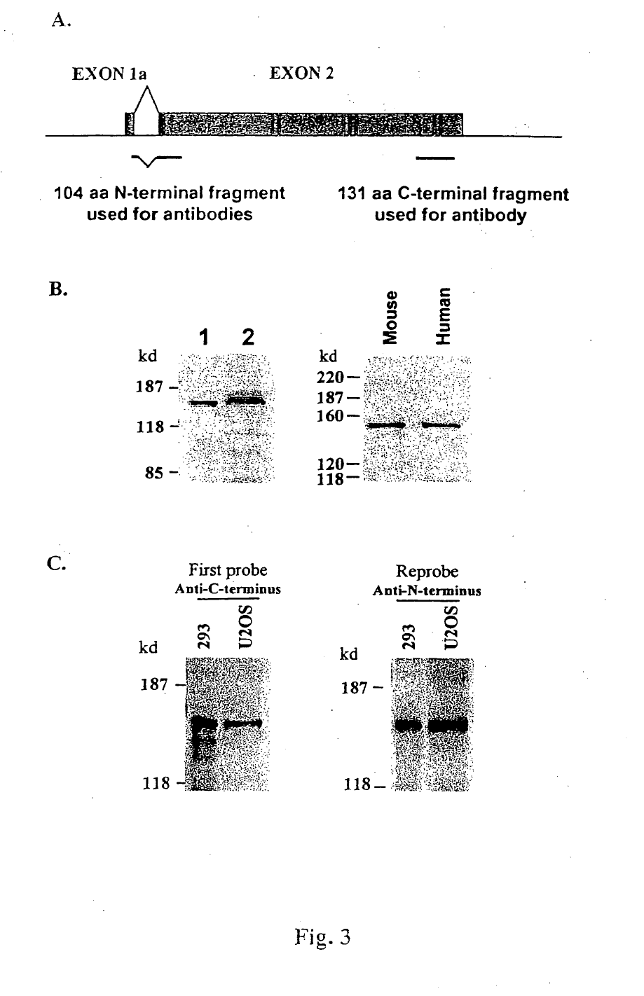

A complete cDNA was obtained using RACE. The sequence was found to be identical to that reported recently for mSal2, with a Glu rather than a Lys residue at position 350. The genomic sequence indicates two alternate short 5′ exons each encoding 24 amino acids and one unique 3′ exon encoding 980 amino acids. The overall homology with hSal2 is 85% using the Blast 2 Sequence program. Eight Zinc fingers are apparent in exon 2. These zinc fingers are organized in four groups with the carboxyl-terminal pair presumed to be an essential part of the large T antigen interaction domain (FIGS. 2B and 3A). FIG. 3A shows the corresponding gene region of the mSal2 protein fragments used to develop antibodies. The exons are boxed, with the zinc fingers represented as stripes.

FIG. 3B shows the antibody detection of in vitro translated full-length mSal2 and p150^{sal2} in mouse and human cells. A polyclonal antibody was made in rabbits against a GST fusion protein containing 131 amino acids from the carboxyl-terminal large T antigen interaction domain. Extracts of mouse 624 and human 293 cell lines probed with this antibody show a single protein species migrating at approximately 150 kDa (FIG. 3B, Right Panel). A monoclonal antibody against a 108 amino acid amino terminal fragment spanning exons 1 and 2 was isolated (FIG. 3A). This antibody also detected mSal2 as a 150 kDa in vitro translation product (Tr), as well as a protein present in normal mouse brain extracts (Br)(FIG. 3B). This gene product of mouse and human origin is referred to as p50. To confirm that the single band from the human cell extract is hSal2, extracts from two human cell lines first were probed with the polyclonal antibody made against the carboxyl-terminus of mSal2. The filter was then stripped and reprobed with the anti-mSal2 amino-terminus polyclonal antibody. The identical band was detected with each of the two antibodies in the human cell lysates (FIG. 3C).

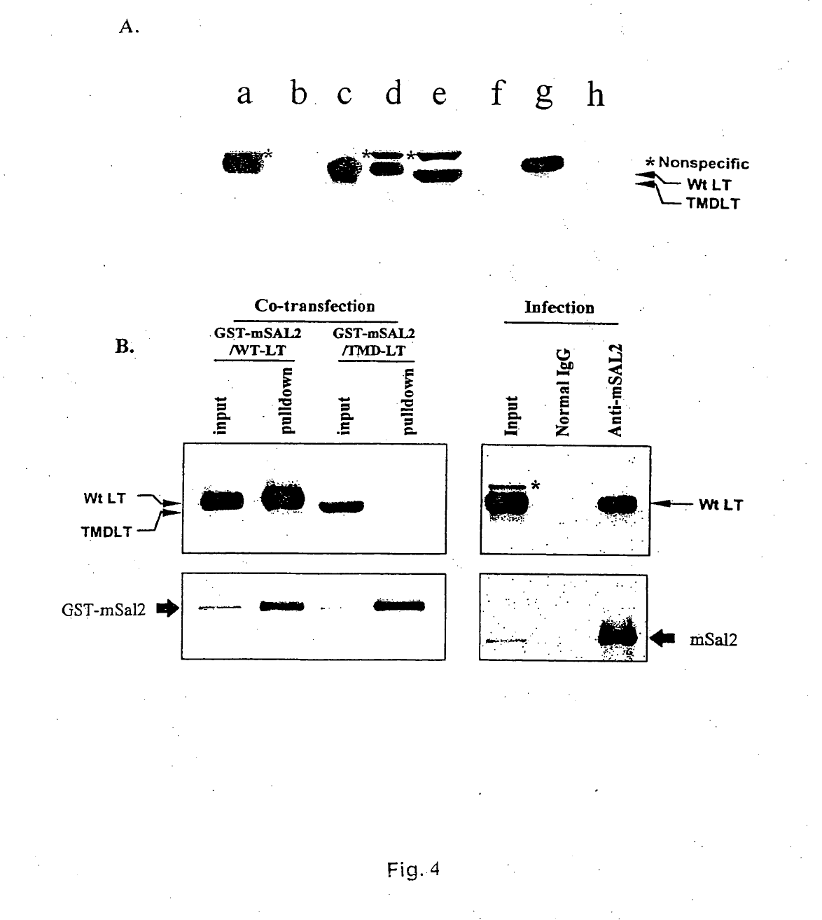

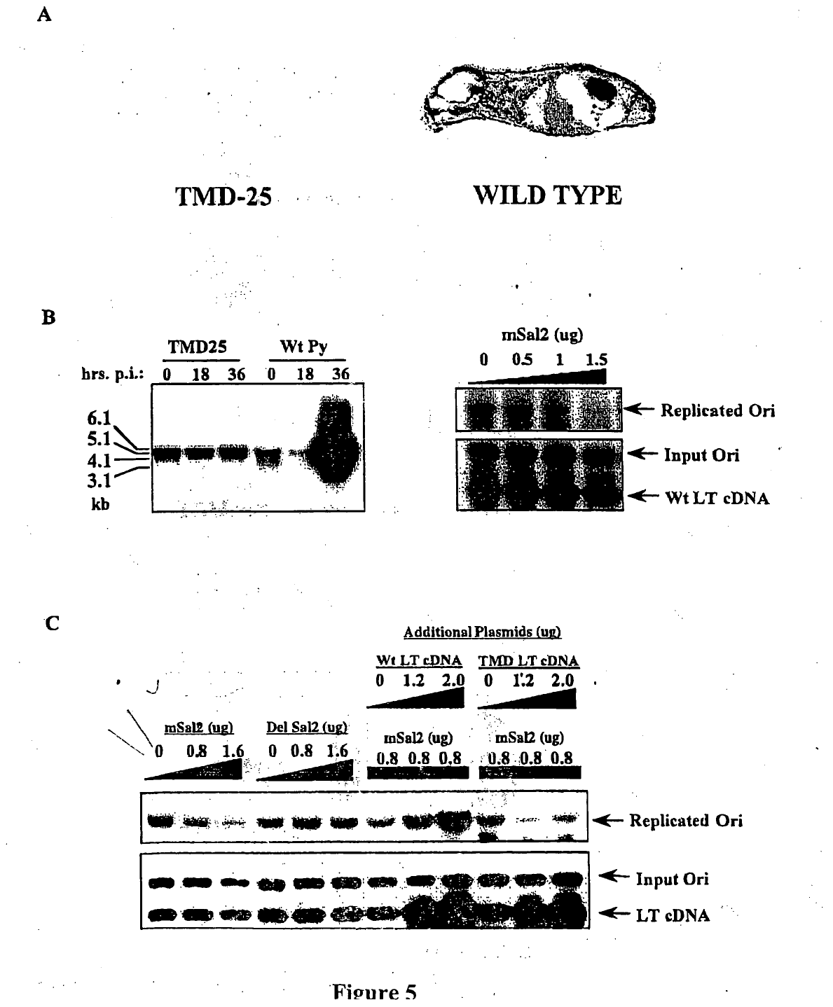

In vitro pull down assays were carried out using a GST fusion of the large T antigen interaction domain of p150^{sal2} and extracts of lytically infected or transfected cells (FIG. 4A). The filter was blotted with an anti-large T antigen antibody. Lanes “a” to “c” show pulldown assays using wild-type polyoma, lytic infected BMK cells: lane “a” shows input extract from normal (WT) Py infected BMK cells; lane “b” shows cell extract from lane “a” pulled down with GST alone; lane “c” shows cell extract from lane “a” pulled down with GST-mSal2 fusion protein. Lanes “d” to “h” show pulldown assays using cell extracts of 3T3 cells transfected with WT large T antigen or TMD25 large T antigen cDNA: lane “d” shows the input extract from 3T3 transfected with WT large T antigen cDNA; lane “e” shows the input extract from 3T3 transfected with TMD25 large T antigen cDNA; lane “f” shows the extract of WT large T antigen cDNA transfected 3T3 cells pulled down with GST alone; lane “g” shows the extract of WT large T antigen cDNA transfected 3T3 cells pulled down with GST-mSal2 fusion protein; and lane “h” shows the extract of TMD25 large T antigen cDNA transfected 3T3 cells pulled down with GST-mSal2 fusion protein. Normal large T antigen synthesized during infection of BMK efficiently binds the GST-mSal2 fragment (lanes a to c). Comparing extracts of 3T3 cells transfected with either wild-type, or TMD-25, large T antigen cDNAs only the wild-type shows binding (lanes d to g).

To confirm the large T-p150^{sal2} interaction in vivo, 3T3 cells were doubly transfected with a vector expressing full length GST-mSal2 and either wild-type, or TMD-25 mutant, large T antigen cDNAs (FIG. 4B Left Panel). Cell extracts were pulled down with glutathione beads. After electrophoresis and transfer, the filter was blotted with anti-large T antigen antibody to show the binding of wild-type or mutant large T antigen. The same filter was blotted again with a monoclonal antibody against mSal2 to show that the level of expression of GST-mSal2 is similar in both the wild-type large T antigen and the TMD25 large T antigen experiments. Each lane is labeled and the input equaled 3% of the extracts used in the co-precipitation assay. Complexes containing normal large T antigen were readily recovered, but no evidence of binding was seen with the mutant large T antigen.

A further experiment was done to confirm the interaction between the large T protein and p150 during a lytic viral infection. An extract of wild-type virus-infected BMK cells was prepared 24 hours post-infection and incubated with polyclonal serum made against the amino-terminal mSal2 fragment. The anti-mSal2 immunoprecipitate was separated and blotted with an anti-T monoclonal antibody. A portion of the large T antigen present in the virus-infected cell extract clearly immunoprecipitated with mSal2, showing that these two proteins interact (FIG. 4B Right Panel). Polyoma large T and p150^{sal2} most likely interact directly through their carboxyl-terminal regions, although additional factors may be involved in mediating the binding.

### Example 4

### TMD-25 is Defective in Virus Growth and Tumor Induction in the Newborn Mouse

Newborn mice were inoculated with either wild-type or TMD-25 mutant virus and followed for development of tumors. The ability of TMD-25 to replicate and spread in the newborn mouse was examined by whole mouse section hybridization (Dubensky et al., J. Virol. 65:342-349 (1991). At ten days post inoculation the mutant showed no signs of replication and spread while the wild-type virus established a disseminated infection with extensive replication in many tissues (FIG. 5A).

Tests for virus replication were carried out on ten-day old animals by whole mouse section hybridization using a ³⁵S-labelled viral DNA probe (FIG. 5A). Newborn mice were inoculated subcutaneously with TMD25 or PTA (1×10⁶ each) and sacrificed ten days later. Frozen sections were probed with ³⁵S labeled viral DNA with overnight exposure. Wild-type PTA showed strong replication in kidney, skin, and bones, while the TMD25 mutant showed no sign of viral replication in any of the organs. Table 2 shows a comparison of tumor induction profile between mSal2 binding mutant TMD25 and wild-type PTA viruses. Newborn mice were inoculated as described above, and sacrificed five months later. Pathological examinations were performed for tumor profile. Wild-type virus rapidly established a disseminated infection and induced a broad spectrum of tumors (Table 2). In contrast, TMD-25 failed to replicate and spread. The only tumors found in mutant-infected mice were subcutaneous fibrosarcomas and these developed only at the site of virus inoculation. Since TMD-25 is defective in replication but retains normal middle and small T functions, these findings are consistent with the expectation that the input mutant virus would be able to infect and transform cells locally but be unable to spread and induce a broad spectrum of tumors.

Direct tests of the mutant's transforming ability were carried out using standard assays with an established line of rat embryo fibroblasts (Dahl et al., Mol. Cell Biol. 16:2728-2735 (1996)). Transformation of these cells does not depend on virus replication, and middle T alone suffices for transformation (Raptis et al., Mol. Cell Biol. 5:2476-2485 (1985)). Mutant virus-infected cells gave rise to foci resembling those induced by wild-type virus; cells derived from one such focus were confirmed, by DNA sequencing, to carry the mutant viral genome. Using DNA transfection followed by measuring colony formation in soft agar, transforming efficiencies were found to be essentially identical for wild-type and mutant viral DNAs—approximately 10-20 colonies/10⁵ cell/μg viral DNA. The failure of TMD-25 to induce tumors at sites distant from the site of inoculation is therefore not due to any defect in transforming ability, but rather to its inability to replicate and establish a disseminated infection.

To investigate whether binding of p150^{sal2} by large T antigen is necessary for viral DNA replication, low molecular weight DNA from BMK cells infected by wild-type or mutant virus was extracted and analyzed by Southern hybridization. The results show clearly that the mutant was unable to replicate its DNA in the non-permissive host (BMK) cells 36 hr post infection (FIG. 5B, Left Panel). BMK cells were infected with TMD25 and wild-type virus (Wt Py). Low molecular weight DNA was isolated at 0, 18, 36 hrs post infection (p.i.) for Southern blot with virus DNA probe. These results suggest that p150^{sal2} can act, directly or indirectly, to inhibit viral DNA replication.

Furthermore, when over expressed in normal 3T3 cells, p150^{sal2} inhibited wild-type viral DNA replication in a dose-dependent manner (FIG. 5B, Right Panel). Polyoma origin clone pUCori (Ori) and large T-expressing plasmid, (Wt LT cDNA), were cotransfected with increasing amount of plasmid expressing mSal2. Newly replicated DNA was detected with origin specific probe (top). The filter was striped and re-probed with LT and origin specific probe to show that similar amount of origin and LT DNA were present in each transfection. These results show that p150^{Sal2} imposes a block to viral DNA replication and that the block can be overcome by wild-type large T antigen.

### Example 5

### Expression Pattern of p150^{sal2} in the Mouse

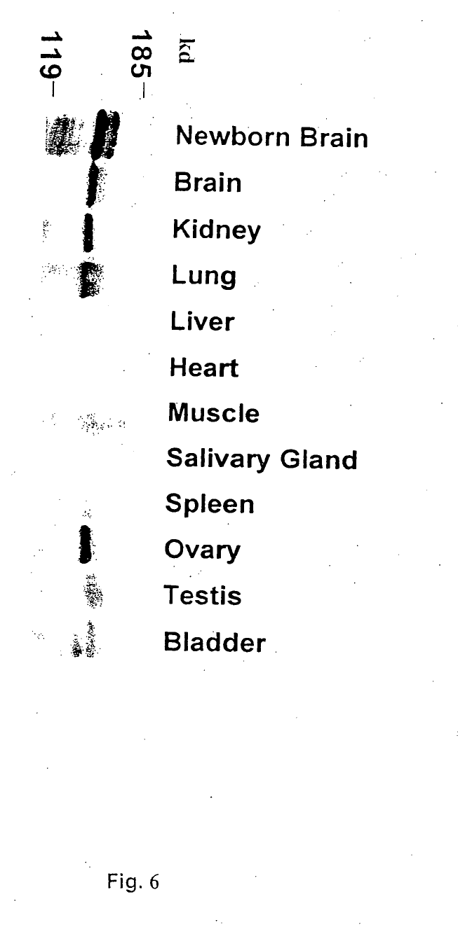

Normal mouse tissues were extracted and tested for expression of p150^{sal2} by Western blot (FIG. 6). Tissues from ten to twelve-day old mice were dissected and extracted in NP-40 lysis buffer. 200 μg of protein from various tissues were loaded onto each lane as labeled. The proteins were detected using a monoclonal antibody against the amino-terminus of mSal2. Tissue from brain, kidney, lung, bladder, and uterus clearly shows expression of the protein, while tissue from liver, skeletal muscle, spleen, salivary gland, and heart was either negative or low in expression. These results are consistent with those reported earlier by Northern analysis. The finding that the kidney and lung are sites of strong expression is also consistent with the natural history of transmission of polyoma, which is thought to infect through the lung and amplify primarily in the kidney. Successful growth in these tissues would require the virus to be able to overcome any block to replication imposed by mSal2. TMD-25 fails to replicate its DNA in normal mouse cells, and overexpression of mSal2 blocks normal viral DNA replication.

### Example 6

### Expression of hSal2 in Human Ovarian Tumors

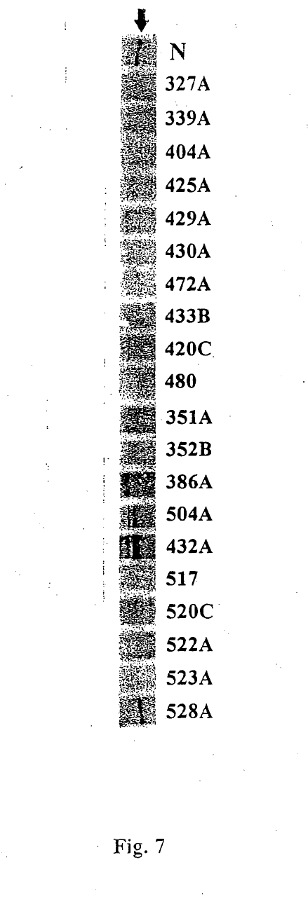

The hSal2 gene has been mapped to chromosome 14q12 but was not recognized initially as a tumor suppressor gene. It was subsequently shown by others that this region of 14q is associated with a loss of homozygosity in 49% of ovarian cancers (Bandera et al., supra) and about 25% of bladder cancers (Chang et al., supra). These findings, along with the underlying rationale of ‘tumor host range’ selection, suggest the possibility that sal2 may function as a tumor suppressor. To test this possibility more directly, a screen for p150 expression was carried out on extracts of ovarian carcinomas, the results of which are summarized in FIG. 7, a Western blot of human ovarian tumors. The expression level of p150^{sal2} in 20 ovarian carcinomas was compared with that of normal ovarian epithelial cells (N) in two panels. Fifty micrograms of protein were loaded in each lane and blotted with polyclonal antibody against p150^{Sal2}. Each ovarian carcinoma was labeled by its case number. Arrows indicate the normal position of p150. A polyclonal anti-p150 antibody made against the mouse protein clearly recognizes the human protein (FIG. 3B above). A band of the same apparent molecular weight is seen in extracts of normal human ovarian epithelial cells (‘HOSE’).

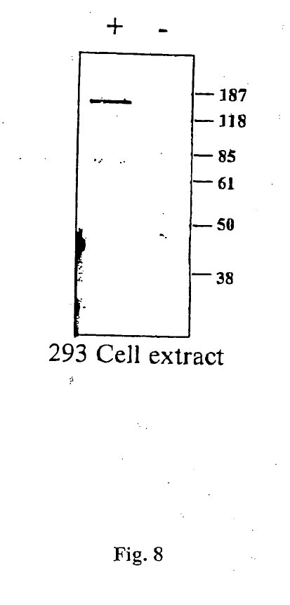

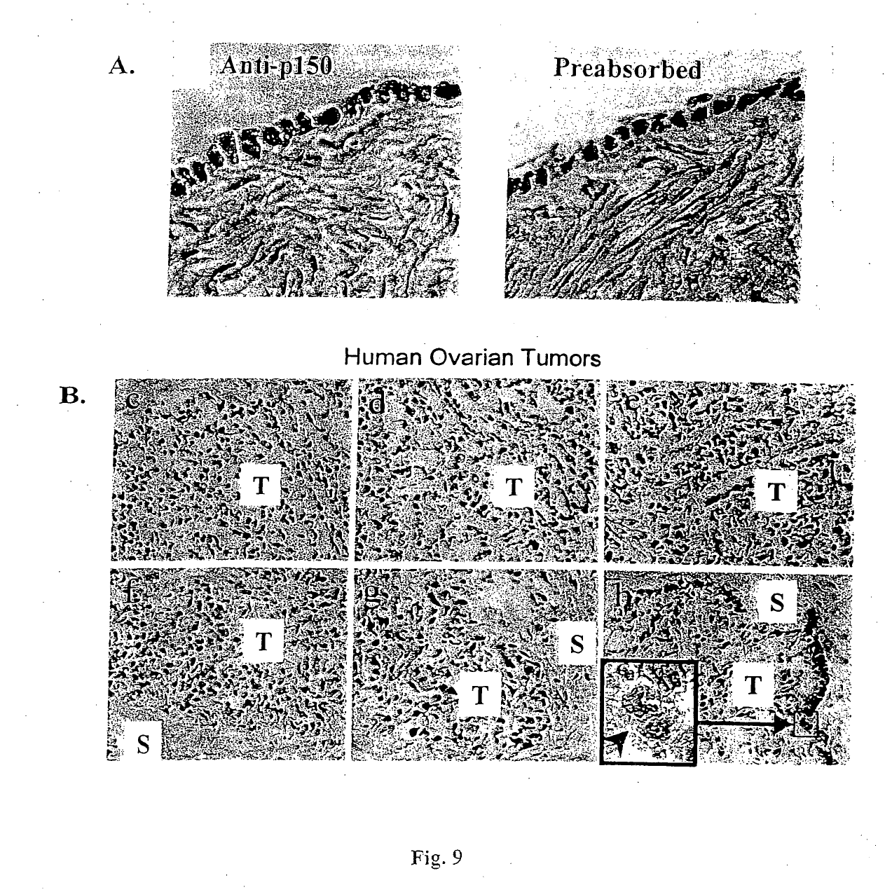

In situ staining with anti-p150 was carried out on frozen sections of normal ovary and several ovarian carcinomas, as well as in human 293 cells. FIG. 8 shows expression of p150^{sal2} in human 293 cells. A polyclonal antibody, HM867, raised against mSal2 carboxyl-terminus, was used to detect human p150^{sal2} in human 293 cells (lane +). As a negative control, the same protein extract was blotted with HM867 antibody that had first been depleted by incubation with the same antigen used to raise it (lane −). As a further example of p150^{sal2} expression, FIG. 9 shows immunostaining of p150^{sal2} in the human ovary and in ovarian tumors. FIG. 9A shows immunostaining of normal human ovarian tissue with a polyclonal serum preadsorbed with mSal2 protein. In the left-hand panel, normal human ovarian tissue is stained with a polyclonal serum preadsorbed with p150^{sal2}. In the right-hand panel, normal ovarian tissue is stained with polyclonal serum against p150^{sal2}. FIG. 9B shows six ovarian carcinoma tissue samples that were stained for p150^{sal2} (c thru h), where “T” stands for tumor cells and “S” stands for stromal cells. The insert in “h” shows cytoplasmic staining for p150^{sal2}. The nuclear staining of normal epithelial cells is readily apparent, but in the ovarian tumor cells the staining is reduced or cytoplasmic.

### Example 7

### A Point Mutation, S73C, in Human Sal2 is Present in Some Ovarian Tumors

DNAs from twenty-one ovarian carcinomas were digested and analysed by Southern hybridization using a probe of hSal2 coding sequences. hSal1 sequences were used as an unlinked internal control. No evidence of loss or gross rearrangement of the hSal2 locus was seen in any of the tumors examined. However, deletions of 1 kb or less would not have been detected. The absence of p150 expression in a majority of ovarian cancers may reflect mechanisms other than loss of the hSal2 gene itself, such as silencing of expression through promoter methylation, alterations in an upstream regulatory factor, or factors leading to instability of the protein itself.

To test for small mutations, DNAs from four tumors were extracted and the entire hSal2 coding regions sequenced on both strands. Two tumors from the panel shown in FIG. 7 that were positive for p150^{sal2} expression and two that were negative were chosen. The two negative tumors 327 and 523 showed no changes when compared to the controls and all showed sequences identical to the published genomic sequence (Genbank AE000658 and AE000521; Boysen et al, Genome Res. 330:330-338 (1997)). The two p150^{sal2}-positive tumors each showed a cysteine (TGT) substitution for serine (TCT) at position 73 (position 73 of SEQ ID NO:1), based on the first methionine in exon 1a (Kohlhase et al., Mamm Genome 11:64-69 (2000). The sequencing results showed only TGT in tumor 432 and a mixture of TGT and TCT in tumor 528. The serine codon TCT has been found at this position in all normal DNAs sequenced thus far (Kohlhase et al., Genomics 38:291-298 (1996); Boysen et al., Genome Res. 330:330-338 (1997)), indicating that ‘73S’ is a frequent normal allele. To know whether the S73C substitution represents a somatic mutation or germ line polymorphism, normal DNA from case 432 was sequenced. The result showed only TGT at codon 73, indicating that the hSal2 allele encoding cysteine represents a germ-line polymorphism in this individual. DNAs from six ovarian carcinoma cell lines were also sequenced and one showed the same S73C substitution as seen in case 432 and another a G744R substitution.

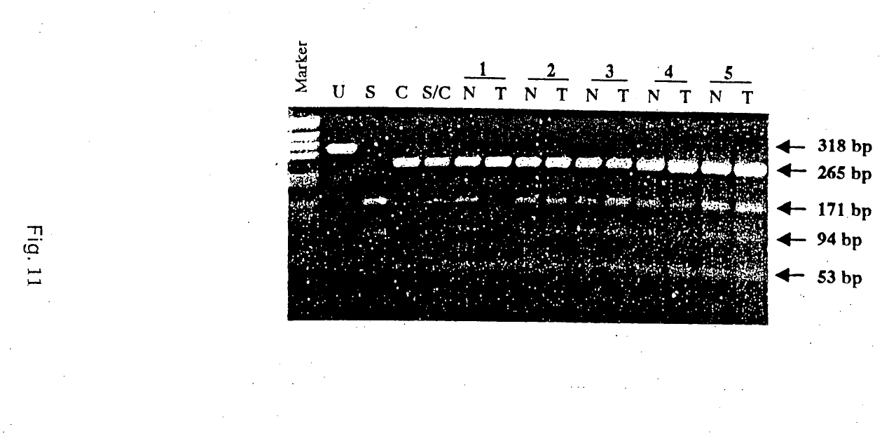

An example of the loss of the 73S allele is shown in FIG. 11. For this experiment, DNA was isolated from matched normal and ovarian tumor tissues. The 73S and 73C alleles were distinguished by PCR amplification and subsequent Mbo II digestion of a 318 bp product covering the region containing amino acid 73. In addition to a common Mbo II site (used to monitor the digestion status), this region contains another Mbo II site for the 73S allele, but not for the 73C allele (this is the discriminating Mbo II recognition site). Complete digestion of 73S allele by Mbo II produced three fragments (171 bp, 94 bp and 53 bp) while 73C allele produced two fragments (256 bp and 53 bp fragments-indicated by arrows). These fragments were resolved by electrophoresis on a 2% agarose gel. Although it is difficult to avoid the existence of normal tissue in the tumor used to isolate DNA, the intensity of the 73S bands (171 bp and 94 bp) is largely reduced indicating the loss of 73S allele (patient number 1). In this figure, “U” indicates undigested amplification product, “S” indicates a 73S homozygote control, “C” indicates a 73C homozygote control, and “S/C” indicates a 73S/C heterozygote control. The respective identification number of ovarian tumor patients is shown on top of their matched normal “N” and tumor “T” DNA.

### Example 8

### mSal2 Suppresses Growth of Ovarian Carcinoma Cells

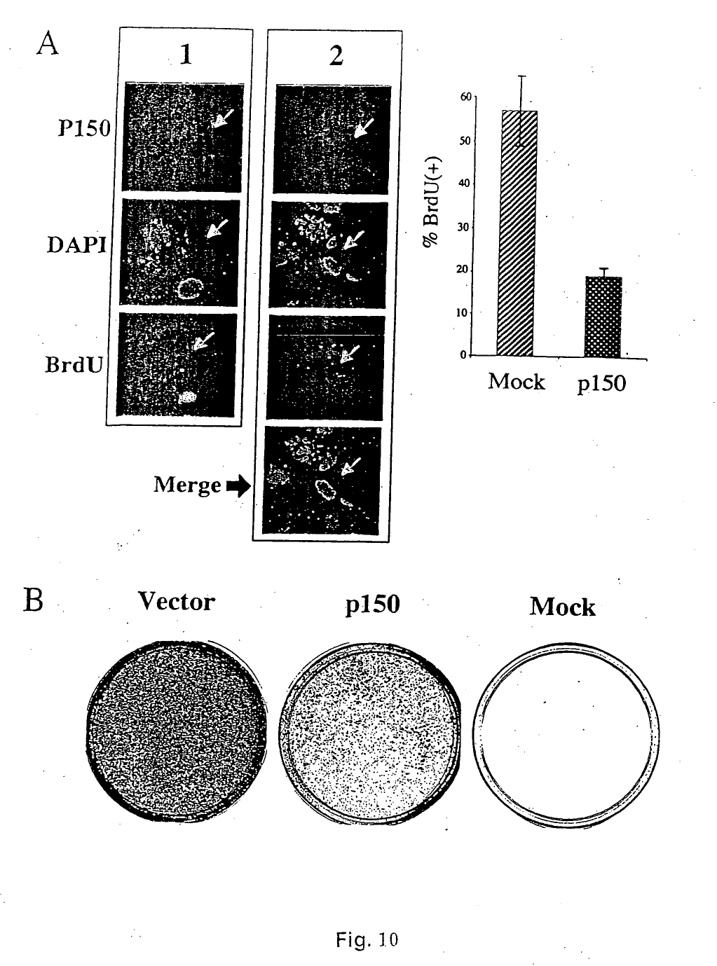

To characterize the biological function of Sal2, the ovarian carcinoma cell line SKOV3 was transfected with an mSal2 expression vector. SKOV3 cells were transfected with pcDNA-mSal2 (P150) or pcDNA3 vector (Mock), incubated in 0.5% serum for 48 hours, then in 15% serum and 100 μM BrdU for 20 hours. This cell line expresses little or no p150^{sal2} as is indicated by Western analysis. Cells were examined by BrdU incorporation for DNA synthesis, for p150 expression, and for DAPI staining (FIG. 10A). The percent of cells in S-phase decreased from 57% in the control to 19% in cells expressing p150. In addition, 30-50% of cells expressing p150^{sal2} appeared to be apoptotic as judged by DAPI staining compared to less than 10% of control cells. Arrows in frame 1 of FIG. 10A indicate a cell expressing p150^{sal2} that is BrdU-negative. Arrows in frame 2 of FIG. 10A indicate an apoptotic cell expressing p150^{sal2} with fragmented nuclear bodies as shown in the merged image. The bar graph in FIG. 10A shows the percentage of BrdU-positive cells in Mock and p150^{sal2} expressing cells. In a colony reduction assay conducted over 14 days, a clear reduction in viable SKOV3 cells was seen in cells transfected with the expression vector, reflecting both growth suppressive, and apoptosis inducing activity of p150^{sal2} (FIG. 10B). Similar efficiencies of transfection (approximately 20%) were confirmed by a co-transfected GFP expression plasmid.

### Example 9

### p150^{Sal2} Expression is Downregulated in Various Cancers

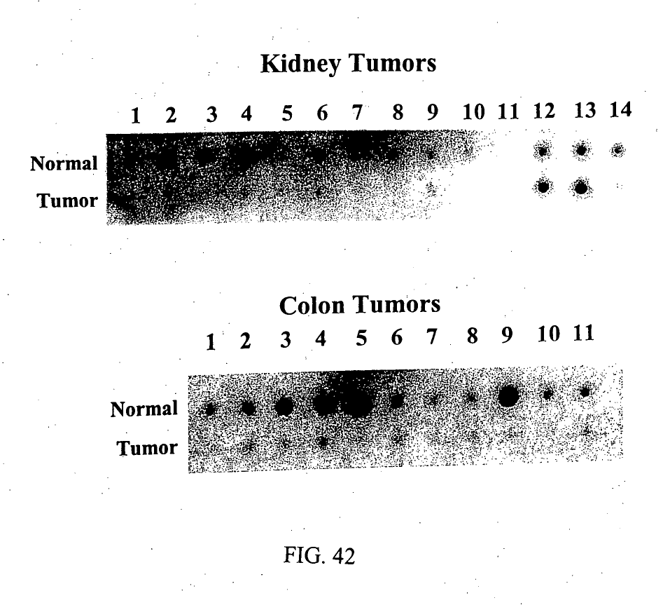

FIG. 42 demonstrates that we have also found p150^{Sal2} to be down regulated in a number of human tumors, in addition to ovarian cancer. Ubiquitin normalized cDNA arrays (Clontech), containing matched normal and tumor tissues from cancer patients, were hybridized with a p150 cDNA (Sal2) to analyze p150 protein expression. Such arrays included the cDNA from 14 samples of kidney tumors and 11 samples of colon tumors. Out of fourteen kidney tumor samples, ten had a marked down regulation of p150^{Sal2} gene product relative to normal control tissues (about 70% of tumors). Similarly, out of eleven of the colon tumor samples tested, eleven showed a down regulation of p150^{Sal2} (100%). Based on this analysis, a down regulation of the protein is clearly associated with colon and kidney cancers.

### Example 10

### p150^{Sal2} Expression Reduces Viral Replication

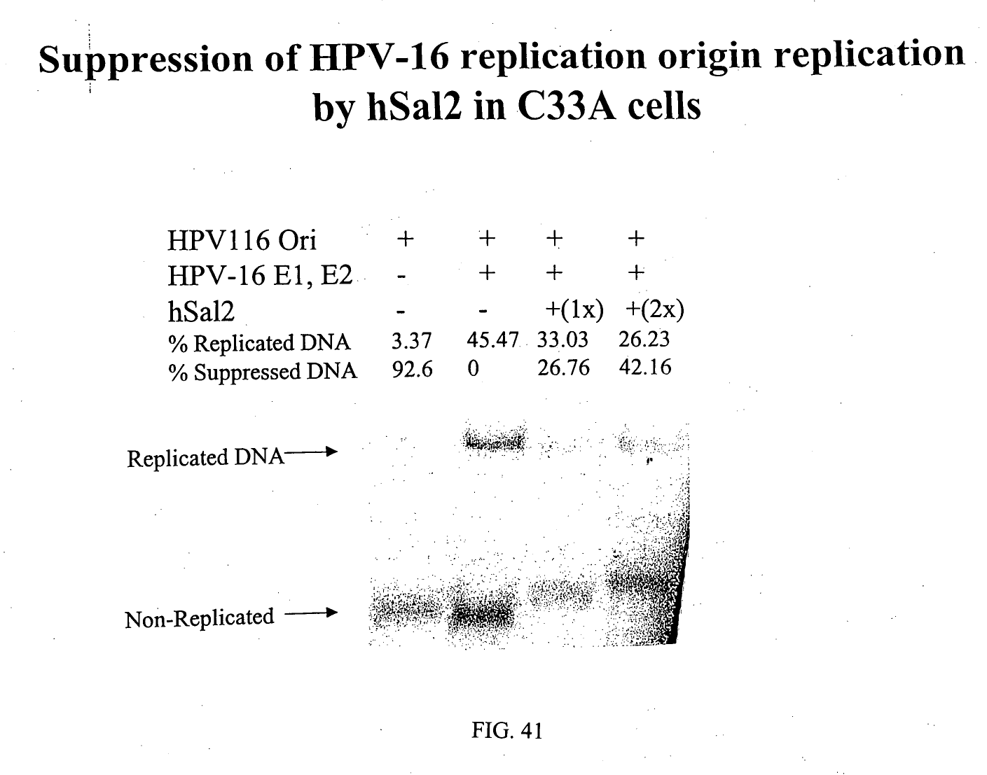

As shown in FIG. 41, the induction in p150^{Sal2} expression can markedly reduce the replication of HPV-16 DNA. C33A cells were transfected with a plasmid containing a viral origin (Ori), which is replicated when the HPV viral replication proteins E1 and E2 are expressed along with the Ori plasmid (second lane, upper band). When p150^{Sal2} is expressed, replication is significantly suppressed (third and fourth lanes, upper band). The amount of suppression is indicated as a percentage based on the ratio of the upper band (replicated DNA) to the lower band (non-replicated DNA) for each lane. According to this finding, a reduction in DNA tumor virus replication and dissemination could be achieved by the administration of p150^{Sal2} to cells infected with a DNA virus.

### Example 11

### p150^{Sal2} is a p53-Independent Regulator of p21^{WAF1/CIP}

As shown above, p150^{Sal2}, a vertebrate homologue of the Drosophila homeotic transcription factor Spalt, is a binding target of the polyoma virus large T antigen. p150^{Sal2} acts as an inhibitor of viral DNA synthesis and the binding of p150^{sal2} by large T overcomes this inhibition. We have further investigated the effects of p150^{Sal2} on the growth and survival of ovarian carcinoma (OVCA) cells that are deficient in expression of p150^{Sal2} and of normal established human ovarian surface epithelial (HOSE) cells which abundantly express the protein. We show that transient expression of exogenous p150^{Sal2} in OVCA cells that show little or no endogenous expression resulted in inhibition of DNA synthesis and colony formation and in increased apoptosis. OVCA cells stably transfected and expressing physiological levels of p150^{Sal2} showed reduced tumorigenicity accompanied by increased expression of p21^{WAF1/CIP1} (p21) and BAX. Conversely, reduction of endogenous levels of p150^{Sal2} in HOSE resulted in reduced p21 expression and increased DNA synthesis. p150^{Sal2} bound to the p21 promoter adjacent to the known p53 binding sites and stimulated transcription in the absence of p53. We propose that p150^{Sal2}, acting in part as a p53-independent regulator of p21 and BAX, can function in some cell types as a regulator of cell growth and survival.

Mammalian p150^{Sal2} (Sal2, SALL2) belongs to the SALL family of proteins homologous to the region-specific homeotic transcription factor Spalt in Drosophila. SALL proteins contain multiple zinc fingers frequently present as C₂H₂ pairs with a conserved linker motif (18). In humans, mutations in SALL genes have been linked to developmental abnormalities. Mice carrying a homozygous knockout of SALL1 die in the early postnatal period with a failure in kidney development. Mice lacking p150^{Sal2} appear to develop normally, although recent observations indicate a defect in myeloid stem cell maturation in these mice similar to that seen in those lacking p21. p150^{Sal2} is differentially expressed in mouse tissues with particularly high levels found in the ovary. Several signaling pathways upstream of SALL genes have been recognized, and these have implications in human cancers.

p150^{Sal2} was identified independently through investigations of the oncogenic murine polyoma virus. As described above, using a ‘tumor host range’ selection procedure designed to uncover cellular factors which become targets for inactivation by the virus, we have shown p150^{Sal2} to be a binding partner of the viral large T antigen (20). In normal mouse cells, p150^{Sal2} has an inhibitory effect on polyoma viral DNA synthesis. The binding of p150^{Sal2} by large T overcomes this inhibition and is an essential step in virus replication and tumor induction. The large T proteins of polyoma and SV40, like oncoproteins of other DNA tumor viruses, function in part by inactivating tumor suppressor genes. Interestingly, while the large T antigens of both viruses bind and inactivate the retinoblastoma tumor suppressor protein pRb, polyoma large T fails to bind and inactivate p53 in the manner of SV40 large T, and conversely, SV40 large T fails to bind p150^{Sal2}. To better understand the molecular and biological functions of p150^{Sal2}, we turn here to studies of ovarian carcinoma (OVCA)-derived cells which are deficient in p150^{Sal2} expression and of established human ovarian surface epithelial (HOSE) cells as the normal precursor which abundantly express the protein.

Our study focuses on the effects of p150^{Sal2} on the growth and survival of ovarian carcinoma (OVCA) cells that are deficient in expression of p150^{Sal2} and of normal established human ovarian surface epithelial (HOSE) cells which abundantly express the protein. Transient expression of exogenous p150^{Sal2} in OVCA cells that show little or no endogenous expression resulted in inhibition of DNA synthesis and colony formation and in increased apoptosis. OVCA cells stably transfected and expressing physiological levels of p150^{Sal2} showed reduced tumorigenicity accompanied by increased expression of p21^{WAF1/CIP1} (p21) and BAX. Conversely, reduction of endogenous levels of p150^{Sal2} in HOSE resulted in reduced p21 expression and increased DNA synthesis. p150^{Sal2} bound to the p21 promoter adjacent to the known p53 binding sites and stimulated transcription in the absence of p53. Thus, p150^{Sal2}, acting in part as a p53-independent regulator of p21 and BAX, may function in some cell types as a regulator of cell growth and survival.

Exogenous p150^{Sal2} Inhibits Growth and Induces Apoptosis in p150^{Sal2}-Deficient OVCA Cell Lines.

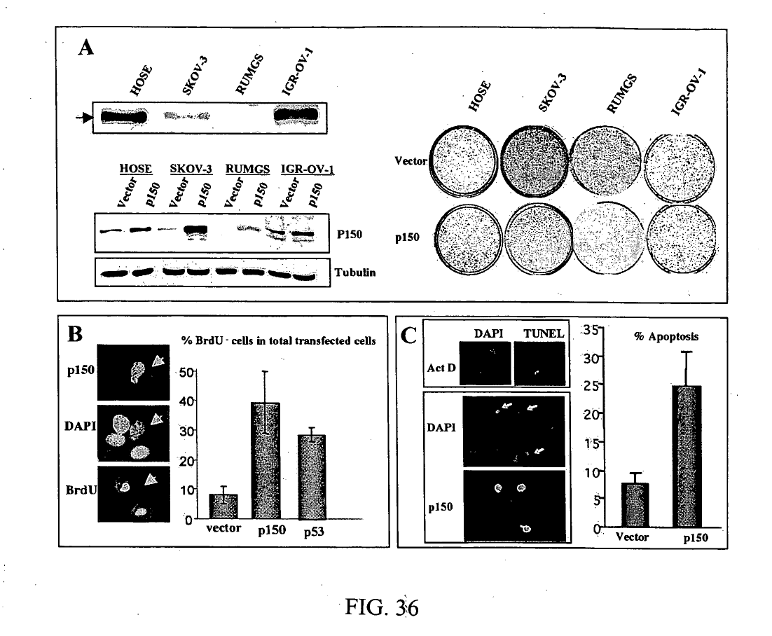

SKOV-3 is a human OVCA-derived cell line that is p53-null and produces tumors in nude mice with a histology similar to that of human ovarian carcinomas. Expression of p150^{Sal2} in SKOV-3 is greatly reduced compared to that in normal established HOSE cells. Two additional OVCA-derived cell lines were found to be either totally lacking (RUMGS) or essentially normal (IGR-OV-1) in p150^{Sal2} expression (FIG. 36A, top left). Transient expression of wild type human p150^{Sal2} in SKOV-3 and RUMGS cells led to reduced colony formation. The growth of ovarian tumor line IGR-OV-1 which expresses endogenous p150^{Sal2} at a normal level was unaffected by exogenous p150^{Sal2} (FIG. 36A, right). Western blots confirmed similar expression levels of p150^{Sal2} constructs following transfection (FIG. 36A, lower left).

Transient expression of exogenous p150^{Sal2} in SKOV-3 resulted in reduced DNA synthesis as indicated by the roughly four-fold increase in cells that fail to incorporate BrdU. A similar degree of inhibition of DNA synthesis was found upon re-introduction of p53 (FIG. 36B). SKOV-3 transfected with p150^{Sal2} also showed a three to four fold increase in apoptotic cells (FIG. 36C). These results demonstrate that p150^{Sal2}, when restored to p150^{Sal2}-deficient OVCA cells, acts in a p53-independent manner to reduce cell replication and viability.

Stable Expression of p150^{sal2} in SKOV-3 Cells Reduces Tumorigenicity

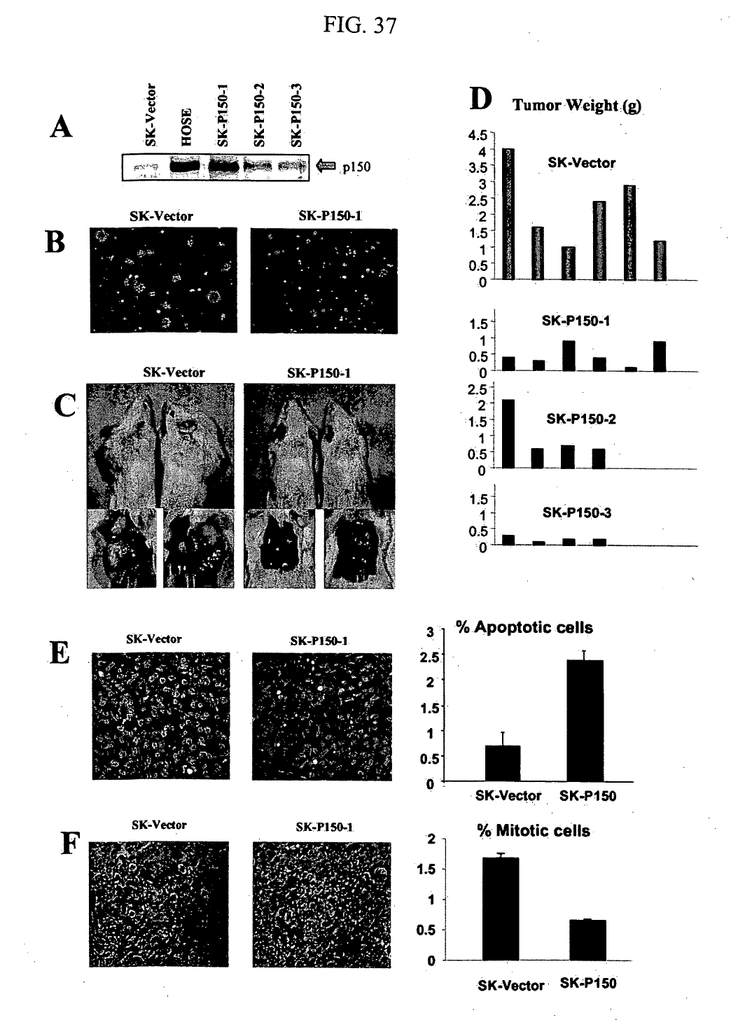

We next attempted to isolate viable clones of SKOV-3 in which expression of p150^{Sal2} was stably restored to approximately normal levels. Three independent clones (SK-P150-1, 2, 3) were isolated showing increased levels of p150^{Sal2} expression, roughly in the range of, but not exceeding, that of normal HOSE cells (FIG. 37A). These SK-P150 cells showed diminished cloning efficiency and reduced colony size in soft agar compared to the empty vector transfected controls cells (SK-Vector) (FIG. 37B). When inoculated subcutaneously into SCID mice, SK-Vector cells produced fast-growing tumors weighing on average 2.2 grams after two months. In contrast, SK-P150 cells produced slower growing tumors averaging 0.6 g (FIGS. 37C and 37D). Apoptotic cells were found at 3-4 fold higher frequency and mitotic cells at 2-3 fold lower frequency in SK-P150 compared to SK-Vector tumors (FIGS. 37E and 37F). The smaller sizes of SK-P150 tumors therefore reflect both pro-apoptotic and growth suppressive effects of p150^{Sal2}.

p150^{Sal2} is a p53-Independent Activator of p21 and BAX

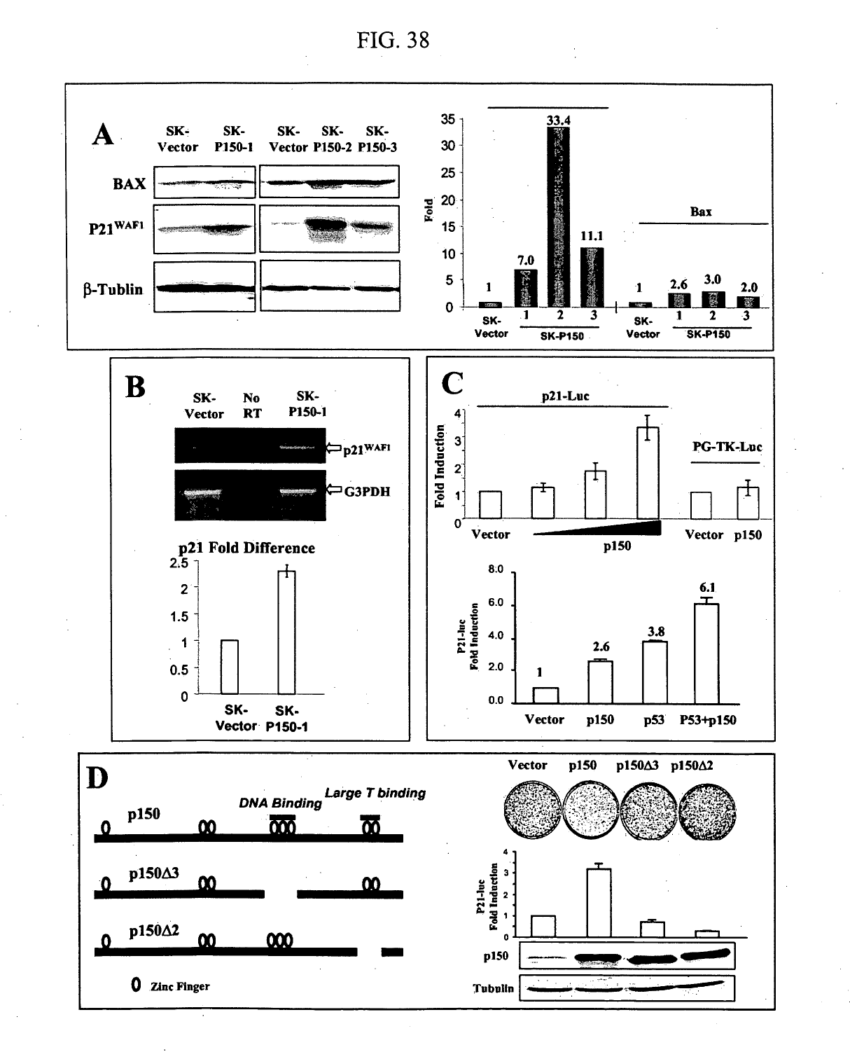

SKOV-3 cells are p53 deficient and can be growth-arrested by exogenous p53. Growth inhibition results largely from p53-dependent transcriptional activation of p21 causing cell cycle arrest and from increased apoptosis mediated by bax. To determine whether the reduction of tumorigenicity by p150^{sal2} is the result of cell growth inhibitory and pro-apoptotic pathways similar to those induced by p53, we compared the expression levels of p53-regulated genes p21 and Bax by Western blot in the three SK-P150 clones and SK-Vector cells (FIG. 38A). Bax protein levels were elevated 2-3 fold in the SK-P150 clones compared to control cells. p21 protein levels were increased more dramatically, in the range of 7-30 fold. P21 transcripts in SK-P150 cells were more than two fold higher in SK-P150 than in SK-Vector cells as determined by RT-PCR (FIG. 38B), indicating that increased transcription contributes to the increased p21 levels in SK-P150 cells.

To examine whether p150^{Sal2} functions directly as a transcriptional activator of p21, p150^{Sal2} expression constructs and a luciferase reporter driven by a 2.7 kb human p21 promoter were co-transfected into SKOV-3 cells. p150^{Sal2} activated the p21 promoter 2-3-fold in a dosage-dependent manner, consistent with the increase in p21 mRNA in SK-P150 cells. p21 promoter activation by p150^{Sal2} is specific since the luciferase reporter driven by a thymidine kinase promoter was not activated by p150 (FIG. 38C, top). Transfection of exogenous wild type p53 stimulated the p21 promoter 3.8 fold compared to 2.6 fold by p150 (FIG. 38C, bottom). When these same amounts of p150 and p53 plasmids were added together, a 6.1 fold induction was observed indicating independent and additive effects on the p21 promoter.

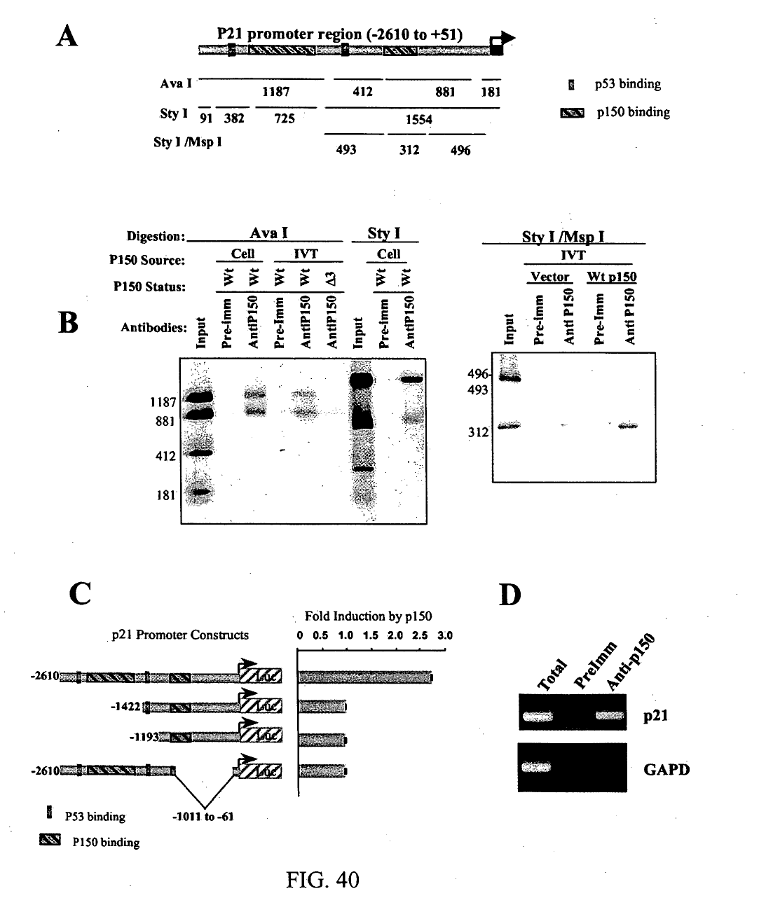

Both the putative DNA-binding (see FIG. 40) and polyoma large T-interaction domains of p150^{Sal2} are essential for transactivation of the p21 promoter. This was shown by transfection of two mutant p150^{Sal2} constructs lacking the triple zinc finger (p150Δ3) and C-terminal double zinc finger (P150Δ2). Though efficiently expressed, neither of these mutant constructs were able to transactivate the p21-Luc reporter (FIG. 38D). As expected, these mutant constructs also failed to inhibit colony formation (FIG. 38D).

Partial Reduction of p150^{Sal2} in HOSE Cells Leads to Increased DNA Synthesis and Reduced Levels of p21

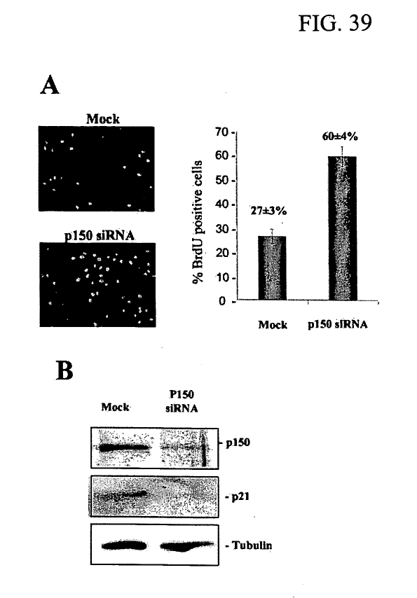

To determine if the maintenance of high levels of p150^{Sal2} is important in the growth regulation of normal HOSE cells, a targeted reduction of p150^{Sal2} was carried out using siRNAs (FIG. 39). Roughly 68 hrs after addition of siRNAs to HOSE cells, the level of p150^{Sal2} was reduced 3 to 4 fold compared to that in control cells. This reduction in p150^{Sal2} was accompanied by a substantial increase in the number of cells synthesizing DNA (FIG. 39A). A roughly 8-fold reduction in p21 level was seen in the same siRNA-treated HOSE cells (FIG. 39B). These results suggest that p150^{Sal2}, acting through p21, can function as a negative regulator of ovarian surface epithelial cell growth.

P150^{Sal2} Binds to the p21 Promoter In Vitro and In Vivo

To determine whether p150^{Sal2} activates p21 transcription by binding directly to the p21 promoter, antibody to p150^{Sal2} was used to immunoprecipitate labeled DNA restriction fragments incubated with p150^{Sal2}. Extracts of P19 cells, which express high levels of endogenous p150^{Sal2}, and in vitro translated p150 cDNA were used as sources of protein. Antibody to p150^{Sal2} but not pre-immune serum immunoprecipitated two non-contiguous DNA fragments from the p21 promoter. The fragments that were bound by p150^{Sal2} flank but do not overlap the two known p53 binding sites (FIGS. 40A and 40B). In vitro translated p150 bound the same fragments as endogenous p150^{Sal2} from the P19 cell extract. p150^{Sal2} contains a triple zinc-finger motif similar to the DNA binding motif of the transcription factor SP1. An in vitro translated p150 with a deletion of this triple zinc finger region (Δ3, amino acids 631-711) was unable to bind the p21 promoter fragments (FIG. 40B). Thus, p150^{Sal2} binds to the p21 promoter directly and the triple zinc finger motif is essential for binding.

To validate the p150^{Sal2} binding regions as functional elements in the p21 promoter, a series of promoter constructs with truncations and internal deletions were transfected into SKOV-3 along with p150^{Sal2} cDNA. Removal of the distal p150^{Sal2} binding region totally abolished inducibility, indicating a p150^{Sal2} responsive element in this region (FIG. 40C). Similarly, an internal deletion of sequences containing the proximal binding region also abolished inducibility. The latter construct responded well to p53, demonstrating that core elements in the promoter proximal region were still present. In this cell context, retention of both p150^{Sal2}-binding regions appears to be essential for a p150^{Sal2}-mediated response.

Further evidence for binding of p150^{Sal2} to the p21 promoter in vivo was sought by chromatin immunoprecipitation (ChIP) using anti-p150^{Sal2} antibody and chromatin from P19 murine embryonal carcinoma cells which express high levels of the protein. Specific amplification of sequences from the p21 promoter was seen using anti-p150^{sal2} antibody but not pre-immune serum (FIG. 40D).

A clue as to the possible molecular function(s) of the murine SALL2 gene emerged in the course of studies of a ‘tumor host range mutant’ of polyoma virus in which p150^{Sal2} was shown to inhibit viral DNA replication. This inhibition is overcome by the viral large T antigen which binds to p150. A mutant virus unable to bind p150^{Sal2} is unable to replicate or induce a broad spectrum of tumors in the mouse. The selection procedure used to isolate this mutant was designed to identify cellular factors that must be altered or inhibited by the virus in order to replicate. Since polyoma replication requires that the virus be able to promote G1→S progression as well as to block apoptosis, it may be expected that p150^{Sal2} would function as a regulator of cell cycle progression and perhaps of apoptosis as well.

Among a number of normal mouse tissues tested for p150^{Sal2} expression, the highest level was found in the ovary. The SALL2 gene has been mapped to human chromosome 14q11-12 (14) adjacent to and possibly overlapping a region of LOH in ovarian cancer. Accordingly, the above experiments were focused on cells derived from human ovarian carcinomas which are deficient in p150^{Sal2} and on ovarian surface epithelial cells as the normal precursors which express high levels of the protein.

Here, we show that p150 is a transcriptional activator of the cyclin-Cdk inhibitor p21, a key factor in G1 checkpoint control. Though the level of induction by p150^{Sal2} of a p21 promoter-Luciferase reporter construct was in the range of 2-3 fold in OVCA cells restored to normal levels of p150^{Sal2} expression, the level of p21 protein achieved in these cells was much higher (7-30 fold). Furthermore, the magnitude of the effects of added p150^{Sal2} were comparable to those of added p53 in terms of p21 induction (FIG. 38C) and reduction in BrdU incorporation (FIG. 36B). p150^{Sal2} also induces apoptosis accompanied by modestly elevated levels of Bax. Whether the pro-apoptotic effect of p150^{Sal2} is mediated all or only in part by Bax is unclear.

The growth suppressive and pro-apoptotic functions of p150^{Sal2} are expressed in the absence of p53. p150^{Sal2} binds to the p21 promoter in vitro and in vivo and stimulates transcription. DNA binding by p150^{Sal2} depends on a triple zinc finger motif, and transactivation requires retention of the C-terminal zinc finger pair which is also essential for binding to polyoma large T antigen. These functions of p150^{Sal2} are accompanied by negative effects on cell growth and survival. They are consistent with predictions of the ‘tumor host range’ selection procedure and also with the general roles SALL genes are thought to play in processes of cell fate determination and terminal differentiation during embryonic development.

The action of p150^{Sal2} in regulating p21 and Bax partially reverses the tumorigenic properties of OVCA cells lacking p53. Introduction of exogenous p150^{Sal2} into OVCA cells led to a sharp reduction in replication and to an increase in the number of apoptotic cells. Clones of p53-negative OVCA cells in which p150 was stably restored to approximately normal levels expressed elevated levels of p21 and Bax and showed substantially reduced growth as tumors in SCID mice. The lower net growth of tumors expressing exogenous p150^{Sal2} was accompanied by a lower mitotic index and an increased apoptotic index. In normal ovarian surface epithelial cells, targeted reduction of the endogenous p150^{Sal2} level led to decreased p21 expression and a concomitant increase in the number of cells synthesizing DNA. Thus the maintenance of p150^{Sal2} levels appears to play a role in regulating the growth of these progenitor cells of OVCA. p150^{Sal2} and p53 appear to function similarly in inhibiting cell replication and in inducing apoptosis. Loss of p53 is common in human ovarian cancer.

p150^{Sal2} may serve a back-up role to p53 in protecting against development of this important type of cancer. This model predicts that expression of SALL2 may be lost or reduced in primary OVCA and that mutations that diminish the ability of p150^{Sal2} to activate the p21 promoter may be found in ovarian tumors.

### Example 12

### Experimental Procedures

Selection of Tumor Host-Range Mutants

The cell lines used as permissive hosts include TCMK-1 (Black et al., Proc. Soc. Exper. Biol. Med. 114:721-727 (1963)) purchased from ATCC) and A6241 (Lukacher et al., J. Exp. Med. 181:1683-1692 (1995); Velupillai et al., J Virology 73: 10079-10085 (1999)). Primary baby mouse kidney cells (BMK) were used as the non-permissive host. The genome of polyoma virus strain PTA was digested at the single BamHI site and cloned into pBlueScript (Stratagene) to create PTAHI. PTAHI was amplified in the Mut D strain of E. coli (Schaaper et al., Proc. Natl. Acad. Sci. U.S.A. 85:8126-8130 (1998)) to accumulate mutations randomly throughout the viral genome.

Yeast Two-Hybrid Screening

The polyoma PTA large T antigen carboxyl-terminal fragment (amino acids 333-781) was cloned into pGBT9 (Clontech) to generate pGBT9IC used as a “bait” to screen a 9.5 to 10.5 day-old whole mouse embryo cDNA library in pVP16 (Vojtek et al., Cell 75:205-214 (1993)). Transformation and selection were performed according to the recommendations from Clontech.

Generation of TMD25 with a Minimum Deletion

Large T antigen carboxyl-terminal deletions used in the yeast two-hybrid analysis were generated on pGBT9ITC using the Tansformer site-directed in vitro mutagenesis kit (Promega) according to manufacturer's recommendations.

Cloning of Full Length mSal2 cDNA

A complete cDNA sequence for mSal2 was obtained by RACE (Frohman) using Marathon cDNA amplification kit (Clontech) and RT-PCR products from BMK cells.

Generation of TMD25 with a Minimum Deletion

Large T antigen carboxyl-terminal deletions used in the yeast two-hybrid analysis were generated on pGBT9ITC using the Transformer site-directed in vitro mutagenesis kit (Promega) according to manufacturer's recommendations.

Cloning of Full Length mSal2 cDNA

A complete cDNA sequence for mSal2 was obtained by RACE (Frohman) using Marathon cDNA amplification kit (Clontech) and RT-PCR products from BMK cells.

RFLP Test to Identify a Polymorphism in Sal2

Amino acid 73 of human p150^{sal2} is polymorphic. This amino acid may be a serine encoded by the codon TCT (73S) or a cysteine encoded by the codon TGT (73C). The two alleles may be distinguished by PCR amplification of the genomic region encompassing the sequence encoding hSal2 amino acid 73 and digesting the PCR product using either the restriction enzyme Mob II or Ear I. These enzymes cut the DNA close to the codon encoding amino acid 73. The primers used to amplify the DNA prior to digestion with Mob II were,

| | |
|---|---|
| 5′-CTTGTTAATTAGAGCCTCGGTATACC-3′ | (SEQ ID NO:7) |
| and | |
| 5′-GCACGGAGGACCCAGAATCTGG-3′. | (SEQ ID NO:8) |

The PCR cycle used was 98° C. for 2 minutes followed by 35 cycles of 94° C. for 1 minute, 55° C. for 1 minute, and 68° C. for 1 minute. After the last PCR cycle, the reaction was incubated at 72° C. for 10 minutes. The PCR products were digested with Mob II in a solution containing 5 μl PCR mixture, 2 μl enzyme buffer (10 fold concentrated), 12 μl water, and 1 μl Mob II (5 units/μl). The restriction digest was performed at 37° C. for two hours followed by heating the reaction to 70° C. for twenty minutes prior to loading ten to twenty microliters of the mixture onto a 2% agarose gel. Five microliters of undigested PCR product were added to a control lane on the gel. The expected size of the uncut PCR product is 318 bp. The expected Mob II restriction fragments for the 73S allele are 171, 94, and 53 bp and the expected Mob II restriction fragments for the 73C allele are 265 and 53 bp. A mixture of the 73S and 73C alleles would be expected to yield fragments of 265, 171, 94, and 53 bp. The 53 bp fragment is common to both alleles and may be used to monitor the digestion status in order to distinguish between heterozygotes and an incomplete digestion.

In Vitro GST Pull-Down Assay

Full-length polyoma normal large T antigen cDNA and TMD25 large T antigen cDNA were cloned into pcDNA3 to create CMVLT and CMVTMDLT respectively. The mSal2 fragment (amino acids 841-971), containing the last zinc finger pair, was cloned into pGEX4T1 (Pharmacia) to generate GST-mSal2 fusion protein in E. coli. The fusion protein was bound to glutathione-Sepharose 4B beads (purchased from Pharmacia) according to the manufacturer's instructions. For the association of GST-mSal2 fusion with large T antigen, BMK cells infected by PTA, or 3T3 cells transfected with wild-type or TMD25 large T antigen expression constructs CMVLT or CMVTMDLT, were extracted with NP-40 lysis buffer (pH 7.9) (Benjamin et al., Proc. Natl. Acad. Sci. U.S.A. 67:394-399 (1970)). 500 μl of cell lysate were incubated with 50 μl of 50% GST-Sal2 or GST beads for 2 hours. After washing four times with PBS, the bound protein was subjected to Western blot analysis using monoclonal antibody F4, which recognizes T antigens (Dahl et al., Mol. Cell. Biol. 16:2728-2735 (1996)).

In Vivo GST Pull-Down Assay

The full-length mSal2 coding region was cloned into a eukaryotic GST fusion vector, pEBG (Luo et al., J. Biol. Chem. 270:23681-23687 (1995)) to generate the construct pEBGSAL. NIH 3T3 cells were co-transfected with pEBGSAL and CMVLT or CMVTNDLT in a ratio of 1 to 1 using Lipofect2000 (Gibco/BRL) according to the manufacturer's protocol. The cells were harvested 48 hours post transfection. The lysate was centrifuged at 3,000 rpm and the supernatant was incubated with 50-100 μl glutathione-Sepharose 4B beads for 2 hours. The beads were washed four times with PBS containing 0.01% NP-40 and the bound proteins were immunoblotted with the F4 antibody and an antibody against p150^{sal2} (Dahl et al., supra (1996)).

In Vivo Co-Immunoprecipitation of mSal2 and Polyoma Large T

Fifty microliters of 50% protein A beads (Pharmacia) were incubated with purified rabbit polyclonal anti-amino-terminal mSal2 antibody or normal rabbit IgG in 1 ml NP-40 lysis buffer at 4° C. for 2 hours, followed by washing the beads four times with PBS. Two milligrams of total protein, made from BMK cells infected with wild-type virus, were extracted 24 hours post infection and incubated with either the anti-mSal2 or normal IgG beads in NP-40 lysis buffer containing 1% BSA for 2 hours at 4° C. After the incubation, the beads were washed four times with 0.1% Tween-20 in PBS and the proteins were separated by SDS-PAGE. Polyoma large T and mSal2 were detected using anti-T and anti-mSal2 monoclonal antibodies.

Viral DNA Replication Assays

Plasmid pUCori and the polyoma origin replication assay are described in Gjorup et al. (Proc. Natl. Acad. Sci. USA 91:12125-12129 (1994)). Cells were grown on 6 well plates and infected with virus or transfected with DNA. Low molecular weight DNA was isolated as described by Hirt (J. Mol. Biol. 26:365-369 (1967)). After purification, the DNAs were resuspended in 80 μl of water. One to five micrograms of DNA were subjected to restriction digestion. For virus infection experiments, the viral genome was first linearized with Eco RI. For transfection experiments, pUCori and CMVLT were first digested with Dpn I and Hind III. The newly synthesized pUCori DNA is Dpn I resistant because of the lack of methylation in eukaryotic cells and the input plasmid DNA is sensitive to Dpn I digestion because of the E. coli methylation of the recognition site. The DNA fragment was resolved on a 1% agarose gel for Southern analysis using origin specific and LT specific probes.

Western Blots for Detection of p150^{sal2}

Tissue extracts were prepared from C3H/BiDa mice by homogenization in NP-40 lysis buffer (pH 7.9) and centrifugation at 8,000 rpm. Fifty micrograms of protein (Bio-Rad Assay) from each sample was separated by SDS-PAGE and blotted on nitrocellulose membranes. A monoclonal antibody against mSal2 was used to detect p150^{Sal2}.

Stripping Western Filters for Reprobing

After first antibody probing, the used filter is incubated in stripping solution (50 mM Tris-Cl, pH 6.8, 2% SDS and 100 mM α-mercaptoethanol) for 30 minutes at 60° C. The filter is washed twice in PBS and tested for the absence of the previously used antibody by development and exposure to an X-ray film. This procedure ensures that the filter can be used again in subsequent Western analyses.

Analysis of Ovarian Carcinomas

Surgical samples of human ovarian tissue were obtained under a protocol approved by the Human Subjects Committee of the Brigham and Women's Hospital. Ovarian tumor tissues were pulverized in liquid nitrogen and lysed in a buffer (1% Triton X-100, 21 μg/ml aprotinin, 1 mM phenylmethylsulfonyl fluoride, 0.5 μg/ml leupeptin, 4.9 mM MgCl₂, and 1 mM vanadate in PBS). The MicroBAC Protein Assay Kit (Pierce) was used for protein quantitation. Twenty-five micrograms of protein from each sample were separated on an SDS-polyacrylamide gel and blotted on nitrocellulose membranes. A rabbit polyclonal antiserum that cross-reacts with hSal2 was used to detect p150^{sal2}. Specifically, this antiserum was raised against a GST-mouse p150^{Sal2} fusion protein that was first purified using Affinity Pak Immobilized Protein A (Pierce) according to manufacturer's instructions followed by an incubation with GST saturated glutathione beads (Pharmacia) in PBS for 30 minutes to eliminate antibodies against GST. As a negative control, the purified antibody was preadsorbed with the GST-p150^{sal2} fusion protein.

Frozen sections of normal or tumor samples were fixed in Neutral Formalin for 10 minutes and permeabilized in cold ethanol/acidic acid (3:1) for 15 min. After washing four times in PBS for 10 minutes each, the sections were antibody stained and processed using Vectastain ABC kit (Vector Laboratories) following the manufacturer's instructions.

DNAs were extracted from human ovarian carcinomas and from primary cultures of ovarian epithelial cells obtained by scraping the surface of normal ovarian tissue. DNA from normal human foreskin was used as a control. The coding region with the 0.4 kb intron of hSal2 was amplified using the primer pair (5′-CCACAACCATGGCGAATCCGAG-3′) (SEQ ID NO:5) and (5′-GGTGATGGAAGGCGAACAGCCAGG-3′) (SEQ ID NO:6). Long range PCR was performed (98° C. 4 min, then 94° C. 1 min, 60° C. 1 min, 68° C. 4 min, for 35 cycles) and sequencing was carried out using the High Throughput Core of the Dana Farber-Harvard Cancer Center. The coding region was sequenced twice and additional sequencing of both strands was performed for regions with suspected mutations. The resulting sequence was compared with the published hSal2 cDNA sequence and genomic sequence.

BrdU Incorporation

SKOV3 cells were transfected with pcDNA-mSal and the pcDNA 3 vector using BRL Lipofectamine 2000 according to the manufacturer's recommendations. Five to seven hours post transfection the cells were fed with 0.5% calf serum. After 48 hours, the cells were incubated with a medium containing 15% calf serum with 100 mM BrdU for 20 to 24 hours. A monoclonal antibody against BrdU (Amersham) was used to detect the incorporation. The cells were fixed, permeabilised and stained according to Amersham's recommendations except that a purified rabbit polyclonal antibody against the mSal2 carboxyl-terminus was mixed with the BrdU antibody for the detection of both BrdU incorporation and p150^{sal2} expression. Secondary antibodies (anti-mouse Rhodamine and anti-rabbit Oregon Green) were also mixed. Cells were examined under fluorescence microscopy in order to identify BrdU and p150^{sal2} positive cells.

Colony Reduction Assay

SKOV3 cells were transfected with a pcDNA-mSal or a pcDNA3 vector in a 6 well plates using 2 μg of DNA each. To monitor the transfection efficiency, 0.5 μg of pEGFPN1 (Clontech) was added to the test DNA in a separate tube. Transfection was performed according to GIBCO/BRL's recommendations using LIPOFECTAMIN 2000. Twenty-four hours after the transfection, the cells were re-seeded in 10 cm plates with medium containing 600 μg/ml G418 (GIBCO/BRL) and 10% calf serum. The EGFP expression was also monitored at this time. The G418 containing medium (neomycin medium) was changed every 3 to 4 days until mock-transfected cells had died and neomycin resistant colonies became apparent.

p50 Expression Constructs

cDNA of mouse p150^{Sal2} from mouse embryo cDNA (Clontech) and human p150 from human fetal brain cDNA (Clontech) were cloned into pcDNA3 (Invitrogen). Deletions of the DNA binding triple zinc fingers (designated Δ3: deletion of aa631-711) and C-terminus large T-binding zinc finger pairs (designated Δ2: deletion of aa911-956) were generated using QuickChange site-directed mutagenesis kit (Stratagene). The p150 stable expression construct was generated using human p150 cDNA cloned into pTet-Splice (Invitrogen).

p21 Promoter Luciferase Constructs

p21 promoter luciferase constructs were derived from p21^{waf1}-Luc. Deletions were produced by either restriction digestion or PCR cloning of desired p21 promoter region into pGL3 basic (Promega).

In Vitro Translation

In vitro translation was performed using the TNT T7 Quick.

Colony Reduction Assay

SKOV-3 or HOSE cells were transfected with mouse or human p150^{Sal2} expression construct as described (20) After 48 hours the cells were seeded in 10 cm plates and selected in 400 μg/ml Geneticin (Invitrogen) for 2 weeks. The colonies were stained with crystal violet and photographed.

Assessment of Apoptosis

SKOV-3 cells were treated with Actinomycin D (5 ng/ml) for 48 hours Cells with characteristic apoptosis DAPI staining were confirmed by Tunnel staining (Intergen). The apoptotic DAPI staining pattern was later used to score apoptosis for SKOV-3 cells in culture. For tumor sections, TUNNEL staining was performed using ApopRed (Intergen) according to manufacturer's suggestions.

BrdU Incorporation Assay

SKOV-3 cells were transfected with p150 expression constructs and pEGFP-N1 (Clontech) in a ratio of 4 to 1. The cells were then growth arrested in 0.5% FBS for 48 hrs and stimulated with medium containing 15% fetal bovine serum and 100 μM BrdU (Sigma) for 20 hours. Cells were stained with antiBrdU kit (Amersham-Pharmacia). The percentage of BrdU negative cells among two hundred GFP positive cells in 4 random fields was plotted.

Stable p150 Elevated Cells

SKOV-3 cells were transfected with human p150^{Sal2} cDNA in vector pTet-splice (Invitrogene) and selected for two weeks in the presence of 400 μg/ml Geneticin (Invitrogen) until resistant colonies formed. The cells were pooled and cloned by limiting dilution. Single colonies were selected by testing the expression of p150 with Western blotting to generate SK-P150 clones. The empty vector transfected cells (SK-Vector) were pooled and used as controls.

Semi-Quantitative RT-PCR

Total RNAs from SK-Vector and SK-P150 clones were amplified using primer pairs (5′-CGT CAC CTG AGG TGA CAC AGC AAA GC-3′ and 5′-CGC TTC CAG GAC TGC AGG CTT CCT G-3′). G3PDH was amplified using (5′-CAG ACC CCA AAT CTG CAG ATA CTC AG-3′ and 5′-CAC TGG AAT TGG AAC TCT TCT GTC GAG-3′). Amplification mixtures from cycle numbers with linear amplification were used for comparing relative quantities of transcripts. Amplified G3PDH cDNA was used as an internal control to normalize the amount of p21 cDNA. The ratio of p21 versus G3PDH is the average of three linear amplification cycles.

Luciferase Assays

p21-Luciferase construct (0.3 μg) was co-transfected with p150^{Sal2} or p53 expression constructs (1.5 μg) and CMV-β-Gal (0.3 μg) (Promega) per ml transfection mixture. For dosage response experiment, 0, 0.5, 1.5 μg p150 construct was used and empty vector was added to makeup the different amount of DNA. Cells were extracted 72 hrs post transfection and assayed for luciferase and β-galactosidase activity. The relative luciferase activity of each sample was normalized by the respective β-galactosidase activity to assess the promoter activity.

Immuno-Precipitation of p150 Target DNA Fragments

This assay has been described previously by McKay et al., Nature 289: 810-3, 1981). The restriction fragments were end-labeled with α-³²P-dCTP by Klenow fragment.

Chromatin Immuno-Precipitation (ChIP)

ChIP was performed as described previously by Weinmann et al., Methods 26:37-47, 2003). P19 cells extracts were used for ChIP. Chromatin elute was amplified using mouse p21 promoter specific primers: 5′-GAA GTA GGA GTC ACC GTC CTG TTT ACC-3′ and 5′-GAT GTC TCT GTA TAG CCC TGG CTG TC-3′ for 45 cycles. As a non-specific control, GAPD (glyceraldehyde-3-phosphate dehydrogenase) gene was amplified with 5′-GCT GAA CGG GAA GCT CAC TGG CAT GG-3′ and 5′-GAG GTC CAC CAC CCT GTT GCT GTA GC-3′.

SiRNA

Two p150 specific siRNA duplexes were made (Dharmacon Research, CO) 5′-AAG GAG AUG GAC AGU AAU GAG-3′ and 5′-AAC CCC AUU ACC UCC AGA AUC-3′ and transfected together into HOSE cells using Oligofectamine (Invitrogen) follow manufacturer's suggestions. The cells were serum-starved (0.2% serum) for 48 hrs and stimulated with 10% FBS and 100 uM BrdU. After 18 hrs, cells were fixed and stained for BrdU and DAPI. p21 was detected by immunoprecipitation followed by Western blot. P150 and tubulin were detected directly by Western blot.

Preparation of DNA for Microinjection

As but one example, DNA clones for microinjection are prepared by cleaving the DNA with enzymes appropriate for removing the bacterial plasmid sequences and subjecting the DNA fragments to electrophoresis on 1% agarose gels in TBE buffer (Sambrook et al. (1989)). The DNA bands are visualized by staining with ethidium bromide and the band containing the desired DNA sequences is excised. The excised band is then placed in dialysis bags containing 0.3 M sodium acetate pH 7.0. The DNA is electroeluted into the dialysis bags, extracted with phenol/chloroform (1:1), and precipitated by the addition of two volumes of ethanol. The DNA is then redissolved in 1 ml of low salt buffer (0.2 M NaCl, 20 mM Tris, pH 7.4, and 1 mM EDTA) and purified on an Elutip-D.™ (Schleicher and Schuell) column. The column is first primed with 3 ml of high salt buffer (1 M NaCl, 20 mM Tris, pH 7.4, and 1 mM EDTA) followed by washing with 5 ml of low salt buffer. The DNA solutions are passed through the column three times to bind the DNA to the column matrix. After one wash with 3 mls of low salt buffer, the DNA is eluted with 0.4 ml of high salt buffer and precipitated by the addition of two volumes of ethanol. DNA concentrations are measured by absorption at 260 nm in a UV spectrophotometer. For microinjection, DNA concentrations are adjusted to 5 μg/ml in 5 mM Tris, pH 7.4 and 0.1 mM EDTA. Other methods for purification of DNA for microinjection are also described in Hogan et al. (Manipulating the Mouse Embryo, Cold Spring Harbor Laboratory, Cold Spring Harbor, N.Y., (1986)); in Palmiter et al. (Nature 300:611 (1982)); in the Qiagenologist, Application Protocols, 3^{rd} edition, published by Qiagen, Inc., Chatsworth, Calif.; and in Sambrook et al. (Molecular Cloning: A Laboratory Manual, Cold Spring Harbor Laboratory, Cold Spring Harbor, N.Y. (1989)). The procedures for manipulation of the rodent embryo and for microinjection of DNA are described in detail in Hogan et al. (Manipulating the Mouse Embryo, Cold Spring Harbor Laboratory, Cold Spring Harbor, N.Y., (1986)), the teachings of which are incorporated herein.

Animal Experiments

Whole mouse section hybridizations (Dubensky et al., J. Virol. 68:342-349 (1991)) and tumor profiles (Dawe et al., Am. J. Pathol. 127:243-261 (1987)) were performed as described in these publications.

Production of Transgenic Mice and Rats

The following is but one preferred means of producing transgenic mice. This general protocol may be modified by those skilled in the art.

Female mice six weeks of age are induced to superovulate with a 5 IU injection (0.1 cc, IP) of pregnant mare serum gonadotropin (PMSG; Sigma) followed 48 hours later by a 5 IU injection (0.1 cc, IP) of human chorionic gonadotropin (hCG, Sigma). Females are placed together with males immediately after hCG injection. Twenty-one hours after hCG injection, the mated females are sacrificed by CO₂ asphyxiation or cervical dislocation and embryos are recovered from excised oviducts and placed in Dulbecco's phosphate buffered saline with 0.5% bovine serum albumin (BSA, Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with humidified atmosphere at 5% CO₂, 95% air until the time of injection. Embryos can be implanted at the two-cell stage.

Randomly cycling adult female mice are paired with vasectomized males. Swiss Webster or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS (Dulbecco's phosphate buffered saline) and in the tip of a transfer pipet (about 10 to 12 embryos). The pipet tip is inserted into the infundibulum and the embryos are transferred. After the transferring the embryos, the incision is closed by two sutures.

The preferred procedure for generating transgenic rats is similar to that described above for mice (Hammer et al., Cell 63:1099-112 (1990). For example, thirty-day old female rats are given a subcutaneous injection of 20 IU of PMSG (0.1 cc) and 48 hours later each female placed with a proven, fertile male. At the same time, 40-80 day old females are placed in cages with vasectomized males. These will provide the foster mothers for embryo transfer. The next morning females are checked for vaginal plugs. Females who have mated with vasectomized males are held aside until the time of transfer. Donor females that have mated are sacrificed (CO₂ asphyxiation) and their oviducts removed, placed in DPBA (Dulbecco's phosphate buffered saline) with 0.5% BSA and the embryos collected. Cumulus cells surrounding the embryos are removed with hyaluronidase (1 mg/ml). The embryos are then washed and placed in EBSs (Earle's balanced salt solution) containing 0.5% BSA in a 37.5° C. incubator until the time of microinjection.

Once the embryos are injected, the live embryos are moved to DPBS for transfer into foster mothers. The foster mothers are anesthetized with ketamine (40 mg/kg, IP) and xulazine (5 mg/kg, IP). A dorsal midline incision is made through the skin and the ovary and oviduct are exposed by an incision through the muscle layer directly over the ovary. The ovarian bursa is torn, the embryos are picked up into the transfer pipet, and the tip of the transfer pipet is inserted into the infundibulum. Approximately 10 to 12 embryos are transferred into each rat oviduct through the infundibulum. The incision is then closed with sutures, and the foster mothers are housed singly.

Generation of Knockout Mice

The following is but one example for the generation of a knockout mouse and the protocol may be readily adapted or modified by those skilled in the art.

Embryonic stem cells (ES), for example, 10⁷ ABI cells, may be electroporated with 25 μg targeting construct in 0.9 ml PBS using a Bio-Rad Gene Pulser (500 μF, 230 V). The cells may then be plated on one or two 10-cm plates containing a monolayer of irradiated STO feeder cells. Twenty-four hours later, they may be subjected to G418 selection (350 μg/ml, Gibco) for 9 days. Resistant clones may then be analyzed by Southern blotting after Hind III digestion, using a probe specific to the targeting construct. Positive clones are expanded and injected into C57BL/6 blastocysts. Male chimeras may be back-crossed to C57BL/6 females. Heterozygotes may be identified by Southern blotting and intercrossed to generate homozygotes.

In addition, knockout mice may also be generated by site-specific recombination methods using, for example, the FLP/FRT system or the Cre-lox system. These systems are described in the specification as well as in, for example, U.S. Pat. No. 5,527,695, Lyznik et al. (Nucleic Acid Research 24:3784-3789 (1996), and Kilby et al. (Trends in Genetics 9:413-421 (1993)).

The targeting construct used in making the knockout animal may result in the disruption of the gene of interest, e.g., by insertion of a heterologous sequence containing stop codons, or the construct may be used to replace the wild-type gene with an altered form of the same gene, e.g., a mutant Sal2 gene. In addition, the targeting construct may contain a sequence that allows for conditional expression of the gene of interest. For example, a sequence may be inserted into the gene of interest that results in the protein not being expressed in the presence of tetracycline. Such conditional expression of a gene is described in, for example, WO 94/29442, WO 96/40892, WO 96/01313, and Yamamoto et al. (Cell 101:57-66 (2000).

TABLE 2
Tumor profiles of mutant TMD-25 and
wild-type PTA virus

| | TMD-25 | PTA¹ |
|---|---|---|
| Fraction of mice with tumors | 7/7 | 32/32 |
| Mean age at necropsy | 202 d. | 82 d. |
| Epithelial tumors: | | |
| Hair follicle | 0/7 | 32/32 |
| Thymus | 0/7 | 29/32 |
| Mammary gland | 0/7 | 16/32 |
| Salivary gland | 0/7 | 23/32 |
| Mesenchymal tumors: | | |
| Fibrosarcomas | 7/7² | 1/32 |
| Renal medulla | 0/0 | 7/32 |
| Bone | 0/0 | 6/32 |

¹Data on PTA is taken from Dawe et al. (Am. J. Pathol. 127: 243-261, 1987).
²Subcutaneous fibrosarcomas were found only at the site of virus inoculation.

TABLE 3
Summary of p150^{sal2} expression in
human ovarian carcinomas

| p150^{sal2} Status | Number of Cases | Percent |
|---|---|---|
| 7 | | |
| Positive | 6 | 30 |
| Negative | 10 | 50 |
| Altered* | 4 | 20 |

*Refers to the apparent size of the Sal2 protein, which is different from that of normal ovarian epithelial cells.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

The disclosures of U.S. Ser. No. 60/216,723, filed Jul. 7, 2000, U.S. Ser. No. 60/339,140 filed Dec. 10, 2001, U.S. Ser. No. 09/988,117, filed on Nov. 16, 2001, U.S. Ser. No. 09/812,633, filed on Mar. 19, 2001, U.S. Ser. No. 10/316,532, filed on Dec. 10, 2002, and U.S. Ser. No. 10/765,520, filed Jan. 27, 2004, and all references cited herein are hereby incorporated by reference

All references cited herein are hereby incorporated by reference.

1. A method of producing a tumor host range (T-HR) mutant virus, wherein said T-HR mutant virus is unable to propagate in normal cells, but is able to propagate in abnormally proliferating cells, said method comprising the steps of:
(a) providing a wild-type viral DNA;
(b) introducing random mutations in said wild-type viral DNA, thereby obtaining a collection of uncharacterized mutant viruses;
(c) infecting abnormally proliferating cells with said collection of mutant viruses to amplify said mutant viruses;
(d) selecting mutant viruses that have the ability to proliferate in said abnormally proliferating cells from said collection by plaque isolation;
(e) infecting normally proliferating cells with mutant viruses selected in step (d);
(f) identifying mutant viruses from step (e) that do not proliferate in said normally proliferating cells, wherein said identified mutant virus are identified as a T-HR mutant viruses.

2. The method of claim 1, wherein said abnormally proliferating cell is uncharacterized.

3. The method of claim 1, wherein said virus has a mammalian host range.

4. The method of claim 3, wherein said mammal is a human.

5. The method of claim 1, wherein said virus is selected from the group consisting of simian virus 40 virus, human polyoma virus, parnovirus, papilloma virus, herpes virus, and primate adenoviruses.

6. The method of claim 1, wherein said T-HR mutant virus identified in step (f) infects a cell in which a tumor suppressor protein is not biologically active.

7. The method of claim 1, wherein said T-HR mutant virus identified in step (f) infects a cell in which an oncogene is expressed.

8. The method of claim 1, wherein said abnormally proliferating cell is a cancer cell.

9. The method of claim 1, wherein said viral DNA provided in step (a) is wild-type viral DNA.

10. The method of claim 1, wherein said collection of mutant viruses obtained in step (b) is uncharacterized.

11. A method of identifying a mammal having or at increased risk of acquiring a proliferative disease, said method comprising the step of determining whether there is a proliferative disease-associated alteration in a Sal2 nucleic acid of said mammal.

12. The method of claim 11, wherein said method is for identifying a mammal having a proliferative disease.

13. The method of claim 11, wherein said method is for identifying a mammal at increased risk of acquiring a proliferative disease.

14. The method of claim 11, wherein said mammal is a human.

15. The method of claim 11, wherein said proliferative disease-associated alteration comprises the substitution of a Cys for the Ser at position 73 of SEQ ID NO:1.

16. The method of claim 11, wherein said determining is done by polymerase chain reaction (PCR) amplification, single nucleotide polymorphism (SNP) determination, restriction fragment length polymorphism (RFLP) analysis, hybridization analysis, or mismatch detection analysis.

17. The method of claim 11, wherein said method comprises the steps of:
(i) contacting a first nucleic acid probe which is specific for binding to said human Sal2 nucleic acid containing said alteration with a nucleic acid from a cell from said mammal under conditions which allow said first nucleic acid probe to anneal to complementary sequences in said cell; and
(ii) detecting duplex formation between said first nucleic acid probe and said complementary sequences.

18. The method of claim 17, wherein said first nucleic acid probe is derived from the human Sal2 nucleic acid containing a proliferative disease-associated alteration.

19. The method of claim 17, further comprising a second nucleic acid probe, wherein said first and second nucleic acid probes are PCR primers, and wherein said human Sal2 nucleic acid or a fragment thereof is amplified using PCR between steps (i) and (ii).

20. The method of claim 17, wherein said cell is from a physiological sample containing abnormally proliferating tissue.

21. The method of claim 17, wherein said cell is from a physiological sample of normal tissue.

22. The method of claim 11, wherein said proliferative disease is cancer.

23. The method of claim 22, wherein said cancer is ovarian cancer.


SEQUENCE LISTING (53 sequences)

SEQ ID NO: 1 - protein, 1005 aa, Homo Sapiens
MAHESERSSRLGVPAGEPAELGGDASEEDHPQVCAKCCAQFTDPTEFLAHQNACSTDPPVMVIIGGQENPNNSSASSEPRPEGHNNPQVMDTEHSNPPDSGSSVPTDPTWGPERRGEESSGHFLVAATGTAAGGGGGLILASPKLGATPLPPESTPAPPPPPPPPPPPGVGSGHLNIPLILEELRVLQQRQIHQMQMTEQICRQVLLLGSLGQTVGAPASPSELPGTGTASSTKPLLPLFSPIKPVQTSKTLASSSSSSSSSSGAETPKQAFFHLYHPLGSQHPFSAGGVGRSHKPTPAPSPALPGSTDQLIASPHLAFPSTTGLLAAQCLGAARGLEATASPGLLKPKNGSGELSYGEVMGPLEKPGGRHKCRFCAKVFGSDSALQIHLRSHTGERPYKCNVCGNRFTTRGNLKVHFHRHREKYPHVQMNPHPVPEHLDYVITSSGLPYGMSVPPEKAEEEAATPGGGVERKPLVASTTALSATESLTLLSTSAGTATAPGLPAFNKFVLMKAVEPKNKADENTPPGSEGSAISGVAESSTATLMQLSKLMTSLPSWALLTNHFKSTGSFPLPLCARALGASPSETSKLQQLVEKIDRQGAVAVTSAASGAPTTSAPAPSSSASSGPNQCVICLRVLSCPRALRLHYGQHGGERPFKCKVCGRAFSTRGNLRAHFVGHKASPAARAQNSCPICQKKFTNAVTLQQHVRMHLGGQIPNGGTALPEGGGAAQENGSEQSTVSGAGSFPQQQSQQPSPEEELSEEEEEEDEEEEEDVTDEDSLAGRGSESGGEKAISVRGDSEEASGAEEEVGTVAAAATAGKEMDSNEKTTQQSSLPPPPPPDSLDQPQPMEQGSSGVLGGKEEGGKPERSSSPASALTPEGEATSVTLVEELSLQEAMRKEPGESSSRKACEVCGQAFPSQAALEEHQKTHPKEGPLFTCVFCRQGFLERATLKKHMLLAHHQVQPFAPHGPQNIAALSLVPGCSPSITSTGLSPFPRKDDPTIP

SEQ ID NO: 2 - DNA, 16080 bp, Homo sapiens
atatcacaccccagctggctatgtaatcatgaaataaggagaaacacataaatatttggttaaaacacctttaatgatagagggaaagacactaatatctcccgtctgttcttgacattttactaggttaggaagctctggagcctacagcttgaggagaagccatcgttcaagtcagtcaatagcaaaaccctcactctctcctcctcagaactcctgttccaaatgatcctatgttaagagtaaatactacaactcattacaagacggagaggcagggaggacgccacctggagctgggactcttaagaaccagacaatgacaaagacacaagccccagcctacggataggcaaaatgggtaggggtcttgaaagaggaagataaggaaaatacaaggggccagggaataaaggagggagttatctaaaactagaagcatactagtgctaggaaatcccccatgatccctggtacacctctgcacactatgtcactattagcccaaaagaatattaacgagaatgtccacattcacaagaatttgaggccttttcccttacatcatgtccctttcttagtcacataggtaccagcaagccctatgttctagcaacattccttaactctctcatcattagttcatcaaccatgctgaccaaaaatgctccttaaagatacgaacttcacatttcccaaatatctcctgggagacctcttggcaagaaatcagcttgtttcccaactttgagaggtcatcatgaatgagaagctggagaggtcttggcacactgaccagccaaaacctttaccttaatgtgaccatcaggggatttactgggaaaattttcctatgcccttccttcatttctccctacttcctagggttgggtcaccaattactggagcatcttcagtaccggcaccttctggagcagggggaggaagaaggaatgtacagtttgctacttcttgtctatgatgggcttctcaggcactgccttgggtgcaggaggctgaaataggaggggggctgtcttctccttggcttccctggatcccattgttggaggcaccttcccagccacagttcctaggccaaacagcactggtggggccaggcttggagtggtagtggaggtggagctggaattccagggcttcatgggcaggccatttgacaggaatgccacatactggttctagaaagataggggacccatacccaccagctgagcagaaaggtcaccccagaggagtggcactgggccctccagagacagctgccagccctttttggctaggctgcaatgccaaatgtaggtgctcaggtgcacctaccaaagggaaagggagaggagagaggagggggaagaagggtcacaccagggaagctggagagggttccccttgagaaagctgcagagaatctatgttcctcaggtacaaagaatgaggagggaagaaaaattccttagggggccatccccttgtaagcacagtaatttccaagctcagggactacagaaaagccactagggacataacatgttaagaacttagagaaaaagacaaaatcagggctcataactctgggaggtccttttgtgaagctgtttctgctctgtgggacaaagagcagcaggtacagaaaaacaggctcatgggatcgtggggtcatcttttcggggaaagggggagagccctgtggaggtgatggaaggcgaacagccagggactagagaaagagcagcaatattctgagggccatggggggcaaagggctgtacctggtggtgtgccaggagcatatgcttcttgagggtagcccgctcaagaaagccctgcctgcagaaaacacaagtgaagagcggcccctccttggggtgggtcttctgatgctcctccagagctgcctgggagggaaaggcctggccacacacttcgcaggcctttctgctgctgctctctcctggctcctttctcattgcctcctgcaggctcagctcctctaccaaggtcacgctggtggcttccccttctggggtgagtgctgatgccggacttgagcttctctccggtttgcccccctcttccttgcctcctaaaacaccactgcttccctgctccattggctgaggctgatccaggctgtcaggtggtggtggtggtggcaaagaagactgttgagtagttttctcattactgtccatctccttcccagctgtggctgctgccgccactgtccccacctcctcctctgccccagatgcctcttctgaatcacctctcactgatattgccttctcacctccactctctgagcctctccctgccagggaatcttcatcagtcacatcttcctcttcttcctcatcctcctcttcctcctcctcagacaactcctcttccggtgatggctgctgggactgctgctgggggaaactccctgccccggagactgtagattgctcggagccattctcctgagcagctcctccaccttcagggagtgcagtaccaccgttggggatctggccccccaggtgcatccggacatgctgctgcagagtgacagcattggtgaacttcttctggcagatggggcaggaattctgtgcccgggcagctggactggccttgtggcccacgaaatgtgcacgcagattacccctggtggagaaggctctgccacacactttgcatttgaagggcctctcacctccatgttggccataatgaaggcgtagggcccgaggacagctaagcactcggagacagatgacacactggttaggtccagaagaggctgaggatgaaggtgcaggggcagaggtggtgggggctcctgaggcagctgaggtcaccgccacagctccttgccggtcaatcttttctaccagttgctgcagctttgatgtctcagagggtgaggcccccaagggctctagcacataggggaaggggaagctgccagtggacttgaagtggttggtaagcagtgcccagcttggtagtgaagtcaccaacttacttagttgcatgcgagttgccgtgctactttctgccactccactgatggctgagccctcactccctgggggggtgttttcatcagctttattcttgggttccactgctttcatgagcacaaacttattgaaagcagggagtcctggagccgtggctgtgcctgcactggtggagagcagagtcaggctctctgtggcactgagtgctgttgtggaggccaccagaggcttgcgctcaacccctccacctggagtggctgcctcctcctcggccttctctggtggcacggacataccataaggcaagccactgctggtaatgacatagtctaggtgctctggtactgggtgtgggttcatctgcacatgtgggtacttctcacgatgccggtggaaatgcactttgaggttgccacgggtggtaaaacggtttccacagacattgcacttatagggcctctcacccgtgtgggaacgaaggtggatctgcagggcactgtcactgccaaatactttggcacagaagcggcatttgtgccttccaccaggcttctccaagggacccatcacttctccgtagctcagctcaccacttccattctttggcttcaggagccctggggaggcagtggcctcaaggcctcgggctgccccaagacactgtgctgccagtagtcccgtggtgcttgggaatgccagatgaggcgaggcaatcagctgatctgtgctgcctggcaaggctggggaaggggcaggggtgggtttgtggcttcgcccaacccctccagcagagaaaggatgctgtgaccccagtgggtggtaaaggtggaagaaggcctgcttgggcgtttctgcccctgaagaggaagaggaggaggaggaggaagatgccagtgtcttgctggtttggacaggcttgatggggctgaagaggggtagtaggggcttggtggaagaggcagtccctgtcccaggtagctctgagggactggcaggggcacccaccgtctggcctaaggagccaagcaacagcacctgcctgcagatttgctcagtcatctgcatctgatggatctgccgctgctgcagcacccgtagctcttccaagatcagggggatattcaagtggccactgcctacccctgggggcggagggggtggtggaggaggagggggtgcaggggtcgattctggaggtaatggggttgctcccagcttgggactggccaagatcaggcccccgcctcccccagccgctgtacctgtggcagcgaccaggaaatgccctggagactcctctcctctcctctctgggccccaggtgggatccgtgggcacggaggacccagaatctggggggttgctatgctctgtgtccatgacctgaggattattgtgaccctcaggccggggttcagaggaggccgaagagttgttggggttctcctggcccccaattatcaccattacaggagggtcagtagaacatgcgttctggtgggcgaggaattcagttgggtcagtgaattgtgcgcagcacttggcacagacttgggggtgatcctcctcgctagcatcacctggggagaagacaaggagagagagcgtgggtggcgcagttgggttgggtataccgaggctctaattaacaaggaggccagtaaccgctagttgggggtggggagatgagctcaccatcagggccatgcagaagtctagagctcaggcctgatccgtgtggacaggagacaacccggcatggggcaggggggtggggagggaggaggggaggggggcaagagcatgctactcccctcctcagccaccctcccttccccaggccacaagcgagttcacggaataggtgtggggacaggggcctacgcagagaatcatgcattttctcccacccaccgaaagtcttcgccgcccctgcgcatccccctccgcccccacccctgcccagcccgaccgaccctaccgcacctccgagctctgccggctccccgcagggcaccccgagacgagagctcctctcggattcgtgcgccatggttgtgggggaagtggagggccaggtggggtgggagacaatggatattgggattgagggaggcgatggccgctgggtctgcggcagcctctgcacccagcggcccagactgcggagatggagatcggcagcggcgggggcagggagcagcggcggagggggaggggagcgaggaggcggggagaagctggagtgagaaagcggggagaggggagatctgggaggagctgatgaggaggggagtttatggggaggagctgctggggagggaggcgggagctagaggaggcgggagaagggagcgctagcgggggcgtgggggcgggagctcagagctcgggagagtttccggaggcgcagtgacaggtgctgtgaagcactgcgggggtccacctttcccggtccctggccagctccccccatctgcagatgcctttgcccaggcctaccctcctccccccgccctcccctcctaagctctaggggcacagtgggaaacgtagccctgctcagtggagcaaggcgataggcttctcttatttttctttggataaaggatccgctgagcttggaaaaagtggattccagagagggtcgtctgatctcctcagaggtctgagggccagaagaagagggggagatcagaacatccactcctcaccagcacacacaccccaaaatattcgaagttttgtctcgtctttctcacttccattcccaccctacccccatccctctccacaaaagaagtttctcagggtgggcggctgcaaggtagaatttcccaggaagtcatttcaggactctctgcggaacactaagccccttcactccccgcccctcctccccctgaataatagctgaatgcaggttactccgcagatcgcccagcctacacaacacctaattcatagagtccatgcttatttaataagccatctcctatttagtaccctcttcctcctctattctcctcttgcaacattcctcacaccgtcactattaaagacagtgggtttggggagacgctagcctgcagaggcctacggaggcccacccagctctaacctgggggggaggggagccctcttgaaacaatgcggtaggaactaccaggcagccctcagtgtctaaagccctttcagccccagcctgatttgaatgcttagaaatagctaacacctgctcaccatcacagaggcagcctcctattcagacaggataagtaagaataaaatgcctcctggaccaggtattctggcattctcttttttaccttgaaatgagtcttaaagtgcttcccacttcctaaaatactttctcttacatgcaggaagtgaccacaagtccttggttttgtggtttccctgggcatcagtaaacctaaattgttttaatcccagttctattcttgcctcactgataaaactgagacatggtggtcagtcacaccatgttataccaccgtttccctcttcataaagtggtaatattgtagctgcagtattttactcagaaaaatattgtggggacaaaaaattgaaaaattggacaatattaatgtgtaaaccaggtatggtggtgcacacttgtagccccagctacttgggaggctgaggcaggagaattgctttagtccaggagtttgaggctgcagtgagctgtgatcacacctgtgaataaccactgccctccagcttcggcaacatagtgaggccccattactttaaaaaaaaaaaaaagccgggcgcggtggctcactgtaatcccagcactttgggaggtgggcagatcacgaggtcagaagttccagaccagcatggccaacatgttgaaaccccgtctctactaaaaatacaaaaattagctgggcatgatggtacacctttaatcccagctactggggcagctgagccaggagaatggcttgaacccaggaggtggaggttgcagggggctgagatcgtggcattgcactccagcctgggcaacaagagtgaaactgcgtctcaaaaaaaaaaaaaagtctaaaaaaattaatatgtacatgtgagatttttaaagtttggggagtcctgaatttaatcaatgagataatttacattgtcagtagcaaaataatcgaagtaaccttaaatacacatatactaaaattagatctgttttccatgttgtttgttaatcttattaatttctgaggtaagatattggctaatatcagcagcatatttcaaaggtaggaagtcttttattgcagtgggtgggggagctgaaacaacctatttaaaatattagtaacatccactttacttctcaacataaattttgcctgtgtttttaaacttaaaacagtttactgaattatgttttgaaacttcagataataaggctcttagcattgtgagtcataattctgaaatggacgggttctgtgcttccaggcctggacttacaaatgagggaggggggttctatttcagtttatggcaagtcacagttttgtgcaatgtggtttatttttacagataaggaaactgaagcttggagaggttaagtgacttttccaagttcacacagtaattcagtgaagcaagcattcagaattttgactcctgtccaatgctttctcaagcacatcaactttgtatggcttccctaatgctagagaaagggccctgtgtggcttctacctgccatttgctccctggccttagtcagggagagggaatcagatggaggctttctactgagcatttgttaattagcattgaacatttgatatccagttgctgttttgtcaagtcttctgacaagaaaagaaatccttttcttttcatcttctcctgggaaacactgtcccctttcttgctctttaatgaaatgtgctttctgatgcgtaatttgatctaagctcttctttaaggtaaatttagtccctggtgaaaggtgactggatcaacagccacctgtaagaggaaccctccatttctcagtactttgcactcactgcacatcctgaaaaggggggcaggattcttacacaaacatgaatgaagtcacaaatgcaggaataaactaaactggtaatggtgtccctagatagcagataaggtgaggtaagctatctccggtcaaatgcaaagtccggggtgggactaagacctggacaagcttgtttaaacttatagagagctgaaatgacaaagaaaagggaaaccaggtggcttcccttctaaatctagtgtcccatcagattgcttctttaggcttcagagagaactgttcgggagaacaaagagaaaaataggtgagttgtatgtagcagggtgatacatttgaacagcggttttcaaattttgctgcccattaggattaccagaagagagttttaaaatttttatgtttaggtgcagtggtctgttccttgtagtcccagctactctggaggctgaggcgggaggatcacttgagctcaagggtttgagactccatctcaaaatctcaaaaaaaagaagaaaaaaaaaagaaaagaaagtttaagcacagtgggtacctcatgcctataatcctagcacttttggaggccaaggcaggaggattgcttgaggccaggaatttgagaccagcctgcataacatagtgagacccccatctctgcaaaagcaaccaaccaaccaaacttaaaaaaaatccctgtgtccaggccacatcccaggctaattaattcataatccctgaggataggatccaggcattagtttgataaagctcctcaggtgattccaatgagaatacaaagatggtgacacaatgatgagacccacatggaggactgccctttccatcataccttccaccctgctcctcacagatcttacctgagctaaacttggccacaattgggacacagacaaaatgaactctcaatgctaaatcttcccatcaggtcccctccctacagtgcccacaaccacacattaacttccttgtatcctttcccagtgaaaaatctgcttccatgaatagaatttgatataatttacaccttactgtaagtttaagtgattgcatttctttcccaggtatgggtatcttgaagcatatttttttcttttttaattgatatttgagccatatttctttttttttctttctttttttttttttttttttttttgagacggagttttgctctcattgcccaggctggagtgcaatggcatgatctcggctcaccgcaacctccacctcccaggttcaagcgattctcctgcctcagccttcccaagtagctgggattacaggcatgtgccaccaagcccggctaattttgtatttttagtagagatggggtttctccatgtgggtcaggctggtctcaaactcccgacctcaggtgatctgccaacctcggcctcccaaagtgttgggattacaggcgtgagccatcgcgcccggccaccatatttctaattgtaaggtgaaaggctttgttctacagagttcaagcatcatccacccattaaggctggagtgaagtggcacaatcatagctcactgcagactctacctcccaggcttaggtgatcctcccacctcagcttcctgaatagctgggactacaggcatgcacaatcatgcccagctaattaaaatatttttttctgtagagatgaggtttcactatgttgcccaggctgtctggaatacctgggctcaagggatcctcctgccttgtccccacaaagtgctgagagtacagatgtaagccactgcctctggcccacttacttattattgacactgaacaatgctaattggtagcttccataattatgaattgattctgtaactattgctactgactacttcttagggaaatatctcatcttctcctccttactcctctttcctaaatgtagacacataataatcctttgcaacccagacctactaatgtaactatggcctatgtaacacagtagactaacaggcacaatgattggtacacctggtgctaagtgagaaaaagatatttgtttccagaacaggaatatcttagatcaaacataagaatgttcttttaatgaaaatttctttgacttcaaaggactcaacacttaacatggaattcataccattttggagctgggacttcagagatctgacactctcattgtcattgtgcacagtgattcagacctgagttaaagtcccagctctagaacattctaatatttgtgatcttgggaaaatttcttaatctctcccagagtttgttttcttatttttttttgggacagagtttcactcttgttgcccaggctggagtgcaatggcacgatcttagctcaccgcaacctccgcctcccaggttcaagcgattctcctgcctcaccctccctagtagctgggattacaggcatgtgccaccacgcccggctaattttgtatttttttagtagagacggggtttcttcatattgctcaggctggtctcaaactcccagcctcaggtgatctgcccacctcggcctcccaaagtgctggattacaggcatgagccaccgcgcctggccagccttttttttttttttgagacggagtctcgctctgtcgcccaggctggagtgcaatggtcgcccaggctggagtgcaatggtgtgatctcggctcactgcaatctccgcctcctgggttcaaacgattttcctgcctcagcctcccaagtagctgggattacaggtgtgcgccatcacacccagctaatttttgtatttttagtagagatgaggtttcaccttgttggccaggctggtcttgaactcctgacctcaagtgatttgcccacctcagcctcccaaagtgctgagattacaggcatgagctgctgtgcccggctgatttctcttctttaaaatgagggtactgccatacaaaggaaggaaattctgatacatgctacaacatgaatgaactttgtaaacattatgctttcagacaaatttgactttaattgagaaaaaaagagaaaacatactaagtgcaataaagcagacacaaaaggacaaatattgtatgattccattagtatgaggtacccaaacattatatgagtccattaatatgaaatttggcaaggtcacacatacagaaagcagagtagaggctaacagggctaagggaatgggagaatggggatttattgtttaacggttacagtttctgtttgatgatgaaaaagatattgaaacagcagtaatggttacataacatagtgaatgtacttaatgccactgaattgtacacttaaaaatggttaaaatggtaaattttattacacatattttacaataaaaaaattttagccaggtgtggtggcatgcacctgtaatcccagctgttcaggaggctgaggcaggagaatctcttgaaccctggaggtggaggtttcagtgagccgagacgtgccactgcactccagcctgggcaacagagtaggacttggtctcaaaaaaagaaaaaaattttttttgtaataataagggagttggggctgggcgtggtggctcacgcctgtaatcccagcactttgggaggccaaagtgggcggatcatgaagtcaggagatcgagaccatcctggctaacacagtgaaaccctgtctctactgaaaatataaaaaattagccaggtgtggtggcgggcgcctgtagtcccagctacttgggaggctgaggcaggagaatggtgtgaacccgggaggcggagctcgcagtgagccaagatcgcgccactgcaccccagcctgggcgacagagcgagactccgtctcaaaataataaaaataaataaataaataaataaaataataataataacggagttgggaggaaaaagaggaaatgcaaaaagggcctagcacagtacctgaatgctccacaaatattagccatgggtgttagttattatttgaatgtcaaaagctgaatgaagccctggggtaagaaaggtcacatgtgcccaaggtcacatagcttcaaggtccacactagattgaaaaccaagttttctgttttcttatctagtactctgtaacaccaggactgagatactctctattccaaaatgtgttttttctgatctgggaatacctaggttgagtggcccagggatcaataacctgagagatgaggctctttacttccaaatgtaaacagagcccccaaaactctacctttgccttctttcctctcttgctgttcttgctatctgccaacttccatctaaagtactcccctctctccctctagatctgtttggctgctgtcctggtttcttcttctcactaaatatctgggtttctgattgtttcctttatttcccagatgtactggtttgcatttttcccccagtcacatcctttgtgttctctaatccagatttctagactctgtaggggagagagaaggtttttttttttcctctctagagtttttaagtgaatagagtatttcctgcccatcacttatatgcaataactgttctgttaggttttgatgctctggttagggaagctgagcaaaaacggctggaaaacagatttttcagactgtttcttggtgatgtcttaggtcactgcagaattttggcttttaaaatatgtaacaaaggctcagcatttgcatgttgtatatggcacatattgcttacaagaaggcaaaagactcctggaaacattactggcaccctagactactgactaaatgtcttctgatactcatgatgatatccataatttcacaggtacaccaaaggatacatgtgcccctaaataagagcccttcctccctaactgtggagcatgctctggggtagaaggaagtcagatgcctgaagatcacataagtgaatagaaaccctgtctataaaaaattagggaaaaggagagctctcattctgttttgcagaatggatgctgccccattcatgattaagaaaatttattaatttaaaagaaaaccagaaaatgtgaaatttatatattataagcttataagatccaggaggaattttagatacgatcaaatagagccacctcattttgcagatgaggcccaatgacatccagatcataagtagcctaggatctttcactccaggggaattctgatgagaaaatccttaggctttcttacggtagatcttaacagagggtgctactgcttccttgctccttacatttgttcctgcctttcatagctcaaaggcaaattttcatcaaaaatttgttgatgccattgggtttaaacctttactgtttctatggggatggctttgtaacagcattaccatgcccccaggtggaagctatatcttaaagggcttgaaaatccattcaagacagccgctaaagatagcttttgactccctcacagaagatttttcctcagctatgatatggggaatgggtgagcagatgggagaagtaggaagaagaggagagaatgcttcttgggggtttggaggggtgttcagcatagttccacaatcaaaccagcaggagagcagaactgtgaggcaactctggggaggagttgaggctctaggggaagtctcctgtagagcacaagcaggaaacatccggcctatagcagcattaagaagggctaatgtgtctcaggagggaaggatgccatcaccatagaacctctaaatatgggcacagtaggatcccagaaaagcagtgtttcggggaggatgcgttctgcccaaaacatgtctgttaaggttattttgtagcacatggagcgctgatttgacctcaagtttttgttttttaacaggtggaaaggcaagtttaatctacaattttagtcgccaccaatacactctcttagagcttttcatgacacgtctcataaagaaatgctgatggccgggagcggtggctcacgcctgtaatcccagcactttgggaggccaaggcgggcagattacgagatcaggagatccagagcatcctggctaacacggtgaaaccccgtctctactaaaaatacaaaaaattagcccggcgtggtggcaggcgcctatagttccagctactcgggaggctgaggcaggagaatggcgtgaacctgggaggtggagcgggcagtgagccaagattgcaccactgcactccaacctgggcgacagagcgagactctctctcaaaaaaaaaaaaagaaaaaaagaaaaagaaaagaaaagaaaaaaaaagaaatgctgacgtttgccaagaggttcctgagttttggtcatactacagcacttgcaggcagtgtcactgcattcacatataatgataataacgatattcacacatattaagcacttatttatgctaggtatttttccaagggatttacacatattaactcatttagattttcacaacaacctaatgaggtagctagtatacacatctttatttcacagatgaggaaactgaagcatagagaggcaaaataaaccagccaaggtcacatagctaaccaagtggtggagctgggatttgtctaaaagtctggtttcagaacccttgtgcttaatcctatactatactgttgggtgtatcaactgtatgctaaacagttgcctgtctggagccaggacttccagactttcagtctgcacatatggagccataccactgacaagtatgtccaaaacttctttgatcctaagaattacctggacaattgcaaaatatatagattcccacaccctggctcagatgtactcacaatcaggcaagtttttaaaacccaggtttagtgggtttagtgagcactaccagccagccctgagcattaggaaattgaagtttttgtcctgattttgcttctgtctctcagactctgagcaatttcactcttcaattccctgcttgctctactgtctgcctgtcacttaacggaatgttacaagaatacatacaatttttccccctcataagggacacctgttgcttcaaaaacacggtatcctcataaaatgatatgcatgtagtaacaggtgtattttcttgcacttcttttgttttgttttgttttttgctttgctttccttgaagcacaaacctaagcccctcatccagacctagccttcagctgtcctccaggtgacacgcatacacaccccaaaccaggctgcattctgaccgaccttagctctctccctctgggagctctgatcggctctcagttcagcccaacaatgagaaacttttttctcgtctccctcaggggagccttcacgtttatccaattcattctcttgcaacccaactctccagaaagaaaaggggggaaaatcccaccccgaagagacggtcttcaggtctgaggacgttacttagcaacggcacaaagaccagtgagcaaagggagacctgaggagaaaactcttgggtggggagacagagccagtttgaaaactccatttcatccagagaaaaacaaggaaaacacaaacagaatcaatcccaagtaacaagcggggcttctccccagcgcaggtcatctcttactccctgcatctcaactccttcaaacccccagtgaccaagtccgcccccgcctggtttcgcccatggcccgagtgccctccccttgccctggcctgacccacacaggcttggacttaggggcccccacccctccccaggcacccaccgttctcagacgcgctgggaccttcgcagtccgagattaactgttggggtttccgctgctttcgccgagacattcccgggtagagagttgggagagggaggggcaacgctcacttggtcttaaccggggtgacctggtctcgtctcccccttgggtccgaagccaattgatgcctctcccccagcgcaaatcactgtgaagcagagatgttcttctttcccagagacacagactctctctctctctctgattctctgttcttgactctctctct

SEQ ID NO: 3 - protein, 1002 aa, Mus musculus
MAQETGSSSRLGGPCGEPAERGGDASEEHHPQVCAKCCAQFSDPTEFLAHQNSCCTDPPVMVIIGGQENPSNSSASSAPRPEGHSRSQVMDTEHSNPPDSGSSGPPDPTWGPERRGEESSGQFLVAATGTAAGGGGGLILASPKLGATPLPPESTPAPPPPPPPPPPPGVGSGHLNIPLILEELRVLQQRQIHQMQMTEQICRQVLLLGSLGQTVGAPASPSELPGTGAASSTKPLLPLFSPIKPAQTGKTTASSSSSSSSSGAEPPKQAFFHLYHPLGSQHPFSVGGVGRSHKPTPAPSPALPGSTDQLIASPHLAFPGTTGLLAAQCLGAARGLEAAASPGLLKPKNGSGELGYGEVISSLEKPGGRHKCRFCAKVFGSDSALQIHLRSHTGERPYKCNVCGNRFTTRGNLKVHFHRHREKYPHVQMNPHPVPEHLDYVITSSGLPYGMSVPPEKAEEEAGTPGGGVERKPLVASTTALSATESLTLLSTGTSTAVAPGLPTFNKFVLMKAVEPKSKADENTPPGSEGSAIAGVADSGSATRMQLSKLVTSLPSWALLTNHLKSTGSFPFPYVLEPLGASPSETSKLQQLVEKIDRQGAVAVASTASGAPTTSAPAPSSSASGPNQCVICLRVLSCPRALRLHYGQHGGERPFKCKVCGRAFSTRGNLRAHFVGHKTSPAARAQNSCPICQKKFTNAVTLQQHVRMHLGGQIPNGGSALSEGGGAAQENSSEQSTASGPGSFPQPQSQQPSPEEEMSEEEEEDEEEEEDVTDEDSLAGRGSESGGEKAISVRGDSEEVSGAEEEVATSVAAPTTVKEMDSNEKAPQHTLPPPPPPPDNLDHPQPMEQGTSDVSGAMEEEAKLEGISSPMAALTQEGEGTSTPLVEELNLPEAMKKDPGESSGRKACEVCGQSFPTQTALEEHQKTHPKDGPLFTCVFCRQGFLDRATLKKHMLLAHHQVPPFAPHGPQNIATLSLVPGCSSSIPSPGLSPFPRKDDPTMP

SEQ ID NO: 4 - DNA, 4547 bp, Mus musculus
atggcgcaggaaaccgggagcagctctcgactcgggggaccctgcggggagcctgcggagcgcggaggtgatgctagcgaggaacaccacccccaagtctgtgccaaatgctgcgcacaattttctgacccgaccgaattcctcgctcaccagaactcatgttgcactgacccaccggtaatggtgataattggaggccaggagaatcccagcaactcttcagcctcctctgcgccccgaccagagggccacagtaggtcccaggtcatggatacagagcacagcaatcccccagattctgggtcctctgggcccccggatcccacttgggggccagagcggaggggagaggaatcttctgggcaattcctggtcgctgccacaggtacagcggctgggggaggtgggggccttatcttggccagtcccaagctgggagcaaccccattacctccagaatccactcctgcaccccctcctcccccaccaccccctccccctccaggtgtaggcagtggccacttgaacattcctctgatcttggaagagttgcgggtgctgcagcagcgccagattcaccagatgcagatgactgaacaaatctgccgccaggtgctgctacttggctccttggggcagaccgtgggtgcccctgccagtccctcagagctacctgggacaggggctgcctcttccaccaagcccctcctgcctctcttcagtcccatcaagccagcgcaaactggcaagacactggcatcttcctcttcgtcatcctcctcctctggagctgaaccgcctaagcaggctttcttccacctttaccatccactgggatcacagcatcctttctctgtaggaggggttgggcggagccacaaacccacccctgccccttcccctgcgctgccaggcagtacggatcagctgattgcttcacctcatctggcattcccaggcaccactggactcctggcagctcagtgtcttggggcagcaaggggccttgaggctgctgcctccccagggctcctgaagccaaagaacggaagtggtgaactgggctatggggaagtgatcagttccttggagaaacccggtggaaggcacaaatgccgcttttgtgcaaaagtattcggcagtgacagcgccctgcagatccaccttcgttcccacactggtgagaggccctataagtgcaacgtctgtggtaaccgtttcacaactcggggcaacctcaaagtacattttcaccggcatcgtgagaagtacccacatgtgcaaatgaatccacatccagtaccggagcacctagactacgtcatcaccagcagtgggctgccttacggaatgtctgtgccaccagagaaagcagaagaggaggcaggcacaccaggcggaggtgttgaacgcaaacccctagtggcctccaccacagcactcagtgccacagagagcctgacactgctctccactggcacaagcacagcagtggctcctgggctccctactttcaacaagtttgtgctcatgaaggcagtggaacccaagagtaaagcggatgagaacacgcccccagggagtgagggctccgccatcgctggagtagcagacagtggctcagcaacccgaatgcagctaagtaagctggtgacgtcactaccgagttgggcactgcttactaatcacttgaagtcaactggaagtttccccttcccttatgtgctagaacccttgggggcttcgccttctgagacctcaaagctgcagcagctagtagaaaagattgaccgccaaggagctgtggcggtggcatctactgcctcgggagctcccaccacttctgcccctgcaccttcctcctccgcttctggacctaaccagtgtgtgatctgtcttcgggtcctgagctgccctcgggctctacgcctgcattatggccaacatggaggtgagcggcccttcaagtgtaaagtgtgtggccgagctttctccacaaggggcaatttgcgcgcacatttcgtgggtcacaagaccagtccagctgcccgggctcagaactcctgccccatttgtcagaagaagttcactaatgctgtcactctgcagcaacatgttcggatgcacctggggggccagatccccaatgggggttccgcactttctgaaggtgggggagctgcccaggaaaacagctctgagcagtctacagcctctggaccagggagtttcccccagccgcagtcccagcagccatctccagaagaggagatgtctgaggaagaggaagaggatgaggaagaggaggaagacgtgacagatgaagattccctagcaggaagaggctctgagagtgggggagagaaggccatatcagtacgaggtgactcagaagaggtatctggggcagaggaagaagtggcaacatcagtagcagcacccaccactgtgaaggagatggacagtaatgagaaagcccctcaacacactctgccgccacctccgccaccacccgacaacctggatcatccccaacccatggagcagggaaccagtgatgtttccggagccatggaggaagaagccaaactggagggaatctcaagcccgatggcagccctcacccaagaaggggagggcaccagcacccctttggtggaagagctgaacttaccggaagccatgaagaaggatccaggagagagcagcggcaggaaggcctgtgaagtatgtggccagagctttcctacccagacagctctggaggagcatcagaagacccatcccaaggatgggccactcttcacttgtgtcttctgcaggcagggcttccttgaccgtgctaccctcaagaagcacatgctgttggctcaccaccaggtaccgccctttgcaccccatggccctcagaatattgctactctttccttggtccctggctgttcctcctccatcccttctccagggctctccccattccctcgaaaagatgaccccaccatgccatgagcctgctttctgtacctggtcctctatgacccagagagcagaaacctgagagcttcatagaggaactccaagatttactcaccctcctcttgtcctttctcaagtcctgacatgatgtttctagtggcttcttctctagtccctgagcttgacaattgcctttgaaagagaatgtccccttaagaaatttttatcacctttttgttctgtgtaactaagggaaacaaattccctatagcttttacattctcaagggggagctctctcctcttctccctttccctttggcaggtatactagaacccccatccttggagtggcagccttggtccaaggggctggcaactgtccatggaaggcccagcgttactccttggtgatcttgaccaccctgcaagactttctagggccgggaccttcttgagaagcttgtaaggggtggtaggtttctttctgcaaccactacccagttttccactgagccctggagttctggacctacctgcattgccactcgggccctagtaccatcattgctgtgaaagcccaggaactgtgtttcacaaggtgactccagtgacatgatccagagaggcaaagaacatagcctccggaagttgaggctgtgcccaacaagcacaccggaagaaagaagaaactataacttctttctccttcccccctgctccagagagtgctggcaataaagatattctagcaattggtgactcaccctagaaggtagggacaagtgaaggactgggaccctttttgcagtatgttccttgactcgccacattgaggcaaagatagtggctggtcaagatgccaggactactccagcttcccatcatgtcctctcaaccaacaagcaggtttcctaccaagaggtctctcgtgtgatagtttagggagtatgaagtttctaactctaaagaatcctgttggtgaggatgattatttaagcaatgatggggagttgagggttgttgctaaaacaggcattgctgggaatctatttgatgaagaacaggacttgatgtaaggggactcgatgttcagctcttgtgagtatgaacgttttctttgagctaatggtgatgtggtatgcagaggtaccaggggccatgggggtgtgtgtgcttcctgtcactagaatgtttttagttttagatgactccctattttattccctcaccccttgtatttcccttgctgtcttctcaaaacccctttcctcccccagttttgcctgaccatgggccagagcttatgtcttattttttttctagaagttgagagacagagcttcaagtggtttccccccgtctctgtcttgtagtgagatgtagtatttactcttaacataggatcctgtggaacaggtgttctgagaagactgaattttgctgttagctgttgtcaatgatgattctctaaagtagtgggctccagagctccctaacacagtgaaatgtgtaagagccgagaggggagatactagaattttttccttcatcattaaaggtgttttggct

SEQ ID NO: 5 - DNA, 22 bp, Artificial Sequence
  derived from human Sal2 gene
ccacaaccatggcgaatccgag

SEQ ID NO: 6 - DNA, 24 bp, Artificial Sequence
  derived from human Sal2 gene
ggtgatggaaggcgaacagccagg

SEQ ID NO: 7 - DNA, 26 bp, Artificial Sequence
  derived from human Sal2 gene
cttgttaattagagcctcggtatacc

SEQ ID NO: 8 - DNA, 22 bp, Artificial Sequence
  derived from human Sal2 gene
gcacggaggacccagaatctgg

SEQ ID NO: 9 - DNA, 63 bp, Polyoma virus
gatatactttgtaatgtgcaagaaggcgacgaccccttgaaggacatatgtgaatatagctga

SEQ ID NO: 10 - protein, 20 aa, Polyoma virus
DILCNVQEGDDPLKDICEYS

SEQ ID NO: 11 - protein, 19 aa, TMD25 mutant Polyoma virus
DILCNVQEDFVMCKKATTP

SEQ ID NO: 12 - DNA, 60 bp, TMD25 mutant Polyoma virus
gatatactttgtaatgtgcaagaagactttgtaatgtgcaagaaggcgacgaccccttga

SEQ ID NO: 13 - protein, 16 aa, Polyoma virus
NVQEGDDPLKDICEYS

SEQ ID NO: 14 - protein, 14 aa, Artificial Sequence
  dervived from Polyoma virus large T antigen
NVQEGDDPLKDICE

SEQ ID NO: 15 - protein, 10 aa, Artificial Sequence
  dervived from Polyoma virus large T antigen
NVQEGDDPLK

SEQ ID NO: 16 - protein, 7 aa, Artificial Sequence
  dervived from Polyoma virus large T antigen
NVQEGDD

SEQ ID NO: 17 - protein, 4 aa, Artificial Sequence
  dervived from Polyoma virus large T antigen
NVQE

SEQ ID NO: 18 - protein, 15 aa, Artificial Sequence
  dervived from Polyoma virus large T antigen
NVQEGDDLKDICEYS

SEQ ID NO: 19 - protein, 15 aa, Artificial Sequence
  dervived from Polyoma virus large T antigen
NVQEGDDPKDICEYS

SEQ ID NO: 20 - protein, 15 aa, Artificial Sequence
  dervived from Polyoma virus large T antigen
NVQEGDDPLDICEYS

SEQ ID NO: 21 - protein, 13 aa, Artificial Sequence
  dervived from Polyoma virus large T antigen
NVQEGDDDICEYS

SEQ ID NO: 22 - DNA, 26 bp, Artificial Sequence
  derived from Polyoma virus large T antigen
cgtcacctgaggtgacacagcaaagc

SEQ ID NO: 23 - DNA, 25 bp, Artificial Sequence
  derived from Polyoma virus large T antigen
cgcttccaggactgcaggcttcctg

SEQ ID NO: 24 - DNA, 26 bp, Artificial Sequence
  derived from Polyoma virus large T antigen
cagaccccaaatctgcagatactcag

SEQ ID NO: 25 - DNA, 27 bp, Artificial Sequence
  derived from Polyoma virus large T antigen
cactggaattggaactcttctgtcgag

SEQ ID NO: 26 - DNA, 27 bp, Artificial Sequence
  derived from Polyoma virus large T antigen
gaagtaggagtcaccgtcctgtttacc

SEQ ID NO: 27 - DNA, 26 bp, Artificial Sequence
  derived from Polyoma virus large T antigen
gatgtctctgtatagccctggctgtc

SEQ ID NO: 28 - DNA, 26 bp, Artificial Sequence
  derived from Polyoma virus large T antigen
gctgaacgggaagctcactggcatgg

SEQ ID NO: 29 - DNA, 26 bp, Artificial Sequence
  derived from Polyoma virus large T antigen
gaggtccaccaccctgttgctgtagc

SEQ ID NO: 30 - DNA, 21 bp, Artificial Sequence
  derived from Polyoma virus large T antigen
aaggagauggacaguaaugag

SEQ ID NO: 31 - DNA, 21 bp, Artificial Sequence
  derived from Polyoma virus large T antigen
aaccccauuaccuccagaauc

SEQ ID NO: 32 - DNA, 3869 bp, Mus musculus
ggggggtttgaaatggcttctcggttaacccgggccagactcaggtatctgctatagaagggaaacaagtgaaagttttcccccccttgcatcatggctcagtttggaggacagaagaatccaccatgggctactcagtttacagccactgcggtctcacaaccagctgcactaggtgttcagcagccatcacttctgggagcatctcctaccatttatacccagcagactgcattggcggcggcaggccttaccacacaaacgccagcaaactatcagttaacacaaactgcggcactgcagcaacaagctgcagctgtattacagcagcaatattcacaacctcagcaggccttgtatagtgtgcagcagcagttgcaacaacctcagcagaccattttaacacagccagctgttgcattgcccacaagccttagcctgtcgactcctcagcctgcagcacagattactgtatcatatccaacaccaaggtccagtcaacagcaaactcaacctcagaagcagcgtgttttcacaggagtggttacaaagctacatgatacatttggatttgtggatgaagatgtattctttcagcttggtgctgttaaagggaaaaccccccaagttggtgatagagtattggttgaagcaacttataatcctaatatgccttttaaatggaatgcacaaagaattcaaacactaccaaatcagaatcagtctcaaacgcaacctttactgaagactccgactgctgttattcagccgattgtgccacagacaacgtttggtgttcaggcacagccccaaccccagtcattattgcaggcccagatctcagctgcctctattacaccactattgcagacgcagccacagcccttattacagcagccacagcagaaagctggtttattgcagcctcctgtccgaatagtgtcacagccacaacctgcgcggagattagatccaccatcacgattttcaggaagaaacgacagaggggatcaagtacctaatagaaaagatgaccgaagtcgtgaaagggacagagaaagacgcagatctagagaaagatcacctcagaggaaacgttcccgggagaggtcaccccggagagaaagagagcgctcccctcggagagtccgtcgtgtcgttccacggtacacagtgcagttttcaaagttttctttagattgtcccagttgtgacatgatggaactaaggcgccgttatcagaacttatatattcctagtgacttttttgatgctcagtttacatgggtggatgctttccctttgtcaagaccatttcaactgggaaattactgcaatttttatgtgatgcaccgagaagtagagtccttagaaaaaaatatggctgttcttgatccacctgatgctgaccacctgtacagtgcaaaggtaatgctgatggctagccctagtatggaagacttgtatcataagtcatgtgctcttgctgaagacccacaagaccttcgtgatggttttcagcatcctgctagacttgttaagtttctagtgggaatgaaaggcaaggatgaagccatggccattggaggccactggtctccttcgctggatggaccaaacccagaaaaagatccctctgtgttgattaaaactgccattcgttgttgtaaggctctgacaggcattgatctaagtgtatgcacacagtggtaccgttttgcagagattcgctaccatcgccctgaggagacccacaaggggcgtacagttccagctcatgtggagacagtggttttatttttcccggatgtttggcattgccttcccacccgctcagagtgggaaaccctctcccgaggatacaagcagcagctggtcgagaagcttcagggtgaacgcaagaaggctgatggagaacaggatgaagaagagaaggatgatggtgaagttaaagagatcgccactcctacccattggtctaagcttgatccaaaggcaatgaaggtaaatgatctccgaaaagaattagaaagtcgagctctcagttccaaaggactaaaatcgcagttaatagctcgcctaacaaagcagcttaaaatagaagaacaaaaagaagagcagaaggaattagagaagtctgaaaaggaagaggaagatgaggatgataagaagtctgaggatgataaagaggaagaagaaagaaaacgtcaagaagaagtggaacgacagcgtcaagaaagaagatacattttgcctgatgaacctgccataattgtgcatccgaactgggctgcaaaaagtggcaagtttgattgcagcatcatgtctttgagtgtccttttggattacagattggaagataataaagaacattcttttgaggtttcactgtttgcagaacttttcaatgaaatgcttcaaagagactttggggttagaatatacaaatcattactctctcttcctgagaaagaggacaaaaaagataaggagaagaaaagcaaaaaagaagagagaaaagataaaaaagaagaaagagaagatgatattgatgaaccaaaaccaaaacggagaaaatcaggcgacgataaagacaaaaaagaagacagagatgagagaaagaaagaagaaaaaagaaaagatgattctaaagatgatgatgaaactgaagaagataacaatcaagatgagtatgacccaatggaggcagaggaagctgaggatgaagatgacgatagggaggaggaggaagtaaaacgagatgacaaaagggatgtcagccggtactgcaaggacagacctgcgaaagataaggaaaaagagaagcctcaaatggtcacagttaacagggatctgctaatggcctttgtttattttgatcaaagtcattgcggttaccttcttgaaaaggatttggaagaaatactatatactcttggactgcatctttcacgggctcaggtaaagaaacttcttaataaagtagtactccgagaatcgtgcttttatcggaaattaacagacacctcgaaagatgatgagaaccatgaagagtcagaggcactgcaggaagacatgctaggaaacagattattacttccaacaccaacaataaaacaggaatcaaaagatggagaggaaaatgtagggcttattgtgtacaatggtgcaatggtggatgttgggagtctcctacaaaaactggaaaagagtgagaaagtaagagctgaggtggaacagaagctccagttactagaggagaaaacagatgaagatgggaaaactatattaaacttggagaactctaacaaaagcctctctggtgaacttagagaggtcaaaaaagaccttggtcaattacaagaaaacctggaggtttcagaaaacatgaatttgcaatttgaaaaccaattgaataaaacactcagaaacttatctacagttatggatgatatccacactgtcctcaaaaaggataatgtaaagagtgaagacagagatgagaaatccaaggagaacggctcaggtgtatgacacagtgcacttggggatgagtgtgttaatagtgtactataaacaaaataatcatgagatgggaatgtttcacggcagtgcatgcttgactttagtagtataaacatatatgttagttcaaatgatgtataaagttttatgaatgtgagtctgcttttgaaaattgcctgtaatttctagcattcaaattattaaatactcactgagtgaagaattttgcattgcaaaaccttttaggatgaacttggttatagtttccccaataaagttcatcagtgtcattgacaatgacaagtaattaaaaccaaaaaaaaaaaaaacaaacaccaaccagg

SEQ ID NO: 33 - protein, 1153 aa, Mus musculus
KFSPPCIMAQFGGQKNPPWATQFTATAVSQPAALGVQQPSLLGASPTIYTQQTALAAAGLTTQTPANYQLTQTAALQQQAAAVLQQQYSQPQQALYSVQQQLQQPQQTILTQPAVALPTSLSLSTPQPAAQITVSYPTPRSSQQQTQPQKQRVFTGVVTKLHDTFGFVDEDVFFQLGAVKGKTPQVGDRVLVEATYNPNMPFKWNAQRIQTLPNQNQSQTQPLLKTPTAVIQPIVPQTTFGVQAQPQPQSLLQAQISAASITPLLQTQPQPLLQQPQQKAGLLQPPVRIVSQPQPARRLDPPSRFSGRNDRGDQVPNRKDDRSRERDRERRRSRERSPQRKRSRERSPRRERERSPRRVRRVVPRYTVQFSKFSLDCPSCDMMELRRRYQNLYIPSDFFDAQFTWVDAFPLSRPFQLGNYCNFYVMHREVESLEKNMAVLDPPDADHLYSAKVMLMASPSMEDLYHKSCALAEDPQDLRDGFQHPARLVKFLVGMKGKDEAMAIGGHWSPSLDGPNPEKDPSVLIKTAIRCCKALTGIDLSVCTQWYRFAEIRYHRPEETHKGRTVPAHVETVVLFFPDVWHCLPTRSEWETLSRGYKQQLVEKLQGERKKADGEQDEEEKDDGEVKEIATPTHWSKLDPKAMKVNDLRKELESRALSSKGLKSQLIARLTKQLKIEEQKEEQKELEKSEKEEEDEDDKKSEDDKEEEERKRQEEVERQRQERRYILPDEPAIIVHPNWAAKSGKFDCSIMSLSVLLDYRLEDNKEHSFEVSLFAELFNEMLQRDFGVRIYKSLLSLPEKEDKKDKEKKSKKEERKDKKEEREDDIDEPKPKRRKSGDDKDKKEDRDERKKEEKRKDDSKDDDETEEDNNQDEYDPMEAEEAEDEDDDREEEEVKRDDKRDVSRYCKDRPAKDKEKEKPQMVTVNRDLLMAFVYFDQSHCGYLLEKDLEEILYTLGLHLSRAQVKKLLNKVVLRESCFYRKLTDTSKDDENHEESEALQEDMLGNRLLLPTPTIKQESKDGEENVGLIVYNGAMVDVGSLLQKLEKSEKVRAEVEQKLQLLEEKTDEDGKTILNLENSNKSLSGELREVKKDLGQLQENLEVSENMNLQFENQLNKTLRNLSTVMDDIHTVLKKDNVKSEDRDEKSKENGSGV

SEQ ID NO: 34 - DNA, 3856 bp, Homo sapiens
gaagttggcgcatgcgcctaaagctgacgggtttgaaatggcttcgatgttagccgggacccgactcagatcgatgctatagaagacaaacaaggaaaggttttttttccttttgcatcatggctcaatttggaggacagaagaatccgccatgggctactcagtttacagccactgcagtatcacagccagctgcactgggtgttcaacagccatcactccttggagcatctcctaccatttatacacagcaaactgcattggcagcagcaggccttaccacacaaactccagcaaactatcagttaacacaaactgctgcattgcagcaacaagccgcagctgcagcagctgcattacaacagcaatattcacaacctcagcaggccctgtatagtgtgcaacaacagttacagcaaccccagcaaaccctcttaacacagccagctgttgcactgcctacaagccttagcctgtctactcctcagccaacagcacaaataactgtatcatatccaacaccaaggtccagtcaacagcaaacccagcctcagaagcagcgtgttttcacaggggtggttacaaaactacatgatacatttggatttgtggatgaagatgtattctttcagcttagtgctgtcaaagggaaaaccccccaagtaggtgacagagtattggttgaagctacttataatcctaatatgccttttaaatggaatgcacagagaattcaaacactaccaaatcagaatcagtcgcaaacccagccattactgaagactcctcctgctgtacttcagccaattgcaccacagacaacatttggtgttcagactcagccccagccccagtcactgctgcaggcacagatttcagcagcttctattacaccactattgcagactcaaccacagcccttattacagcagcctcagcaaaaagctggtttattgcagcctcctgttcgtatagtttcacagccacaaccggcacgacgattagatcccccatcccgattttcaggaagaaatgacagaggggatcaagtgcctaacagaaaagatgatcgaagtcgtgagagagagagagaaagacgtagatcgagagaaagatcacctcagaggaaacgttcccgggaaagatctccacgaagagagcgagagcgatcacctcggagagttcgacgtgttgttccacgttacacagttcagttttcaaagttttctttagattgtcccagttgtgacatgatggaactaaggcgccgttatcaaaatttgtatatacctagtgacttttttgatgctcaatttacatgggtggatgctttccctttgtcaagaccatttcagctgggaaattactgcaatttttatgtaatgcacagagaagtagagtccttagaaaaaaatatggccattcttgatccaccagatgctgaccacttatacagtgcaaaggtaatgctgatggctagccctagtatggaagatttatatcataagtcatgtgctcttgctgaggacccacaagaacttcgagatggattccaacatcctgctagacttgttaagtttttagtgggcatgaaaggcaaggatgaagctatggccattggaggccactggtctccttcgttggatggaccagacccagaaaaagatccctctgtgttgattaagactgctattcgttgttgtaaggctctgacaggcattgatctaagtgtgtgcacacaatggtaccgttttgcagagattcgctaccatcgccctgaggagacccacaaggggcgtacagttccagctcatgtggagacagtggttttatttttcccggatgtttggcattgccttcccacccgctcagagtgggaaaccctctcccgaggatacaagcagcagctggtcgagaagcttcagggtgaacgcaaggaggctgatggagaacaggatgaagaagagaaggatgatggtgaagctaaagaaatttctacacctacccattggtctaaacttgatccaaagacaatgaaggtaaatgacctccgaaaagaattagaaggtcgagctcttagttccaaaggattaaaatcccagttaatagcccgattgacaaaacagcttaaagtagaggaacaaaaagaagaacagaaggagttagagaaatctgaaaaagaagaggatgaggatgatgataggaaatctgaagacgataaagaggaagaagaaaggaaacgtcaagaggaaatagaacgccagcgtcgagaaagaagatatattttgcctgatgaaccggccatcattgtacatccaaattgggctgcaaaaagtggcaagtttgattgtagcatcatgtctttgagtgtcctattggactacagattagaggataataaagaacattcatttgaggtttcattgtttgcggaacttttcaacgaaatgcttcaaagagattttggtgtccgtatatacaaatcattactgtctcttcctgagaaagaggacaaaaaagaaaaggataaaaaaagcaaaaaagatgagagaaaagataaaaaagaagaaagagatgatgaaactgatgaaccaaaacccaaacggagaaaatcaggcgatgataaagataaaaaagaagatagagatgaaaggaagaaagaagataaaagaaaaggtgattctaaagatgatgatgaaactgaagaagataacaatcaagatgaatatgaccctatggaagcagaagaagctgaggatgaagaagatgatagggatgaggaagaaatgaccaaacgagatgacaaaagagatatcaacagatactgcaaggagaggccctctaaagataaggaaaaagaaaagactcaaatgatcacaattaacagagatctgttaatggcttttgtttattttgatcaaagtcattgtggttaccttcttgaaaaggatttggaagaaatactttatactcttggactacatctttctcgggctcaggtaaagaagcttcttaataaagtagtgctccgtgaatcttgcttttaccggaaattaacagacacctcaaaagatgaagagaaccatgaagagtctgagtcattgcaggaagatatgctaggaaacagattattacttccaacaccaacagtaaagcaggaatcaaaggatgtggaagaaaatgttggcctcattgtgtacaatggtgcaatggtagatgtaggaagcctcttgcaaaaattggaaaagagcgaaaaagtaagagctgaggtagaacagaagctgcagttactagaagaaaaaacagatgaagatgaaaaaaccatattaaatttggagaattccaacaaaagcctctctggtgaactcagagaagttaaaaaggaccttagtcagttacaagaaaacttaaagatttcagaaaacatgagtttacaatttgaaaaccaaatgaataagacaatcagaaacttatctacggtaatggatgaaatccacactgttctcaagaaggataatgtaaagaatgaagacaaagatcaaaaatccaaggagaatggtgccagtgtatgataaaatccatgtagtgatgaggaatggtgttaaataatgtaatatataaaaatcatgatataagaatgtttgaaggtgatgcatgtttgattttagtagtataaatgtattttagttcaaatgatgtataaagttttatgaatgtgagtttctgcttttgaaaattgcttgtaattcctagccttcaaattattaaacactccttgagtgaaataattttgcattgcaaagtgttttaggatgaactttgttatagttttaactccaataaagttcatcagttt

SEQ ID NO: 35 - protein, 1172 aa, Homo sapiens
PGPDSDRCYRRQTRKGFFSFCIMAQFGGQKNPPWATQFTATAVSQPAALGVQQPSLLGASPTIYTQQTALAAAGLTTQTPANYQLTQTAALQQQAAAAAAALQQQYSQPQQALYSVQQQLQQPQQTLLTQPAVALPTSLSLSTPQPTAQITVSYPTPRSSQQQTQPQKQRVFTGVVTKLHDTFGFVDEDVFFQLSAVKGKTPQVGDRVLVEATYNPNMPFKWNAQRIQTLPNQNQSQTQPLLKTPPAVLQPIAPQTTFGVQTQPQPQSLLQAQISAASITPLLQTQPQPLLQQPQQKAGLLQPPVRIVSQPQPARRLDPPSRFSGRNDRGDQVPNRKDDRSRERERERRRSRERSPQRKRSRERSPRRERERSPRRVRRVVPRYTVQFSKFSLDCPSCDMMELRRRYQNLYIPSDFFDAQFTWVDAFPLSRPFQLGNYCNFYVMHREVESLEKNMAILDPPDADHLYSAKVMLMASPSMEDLYHKSCALAEDPQELRDGFQHPARLVKFLVGMKGKDEAMAIGGHWSPSLDGPDPEKDPSVLIKTAIRCCKALTGIDLSVCTQWYRFAEIRYHRPEETHKGRTVPAHVETVVLFFPDVWHCLPTRSEWETLSRGYKQQLVEKLQGERKEADGEQDEEEKDDGEAKEISTPTHWSKLDPKTMKVNDLRKELEGRALSSKGLKSQLIARLTKQLKVEEQKEEQKELEKSEKEEDEDDDRKSEDDKEEEERKRQEEIERQRRERRYILPDEPAIIVHPNWAAKSGKFDCSIMSLSVLLDYRLEDNKEHSFEVSLFAELFNEMLQRDFGVRIYKSLLSLPEKEDKKEKDKKSKKDERKDKKEERDDETDEPKPKRRKSGDDKDKKEDRDERKKEDKRKGDSKDDDETEEDNNQDEYDPMEAEEAEDEEDDRDEEEMTKRDDKRDINRYCKERPSKDKEKEKTQMITINRDLLMAFVYFDQSHCGYLLEKDLEEILYTLGLHLSRAQVKKLLNKVVLRESCFYRKLTDTSKDEENHEESESLQEDMLGNRLLLPTPTVKQESKDVEENVGLIVYNGAMVDVGSLLQKLEKSEKVRAEVEQKLQLLEEKTDEDEKTILNLENSNKSLSGELREVKKDLSQLQENLKISENMSLQFENQMNKTIRNLSTVMDEIHTVLKKDNVKNEDKDQKSKENGASV

SEQ ID NO: 36 - protein, 18 aa, Mus musculus
GGFEMASRLTRARLRYLL

SEQ ID NO: 37 - protein, 4 aa, Mus musculus
KGNK

SEQ ID NO: 38 - protein, 12 aa, Mus musculus
HSALGDECVNSV

SEQ ID NO: 39 - protein, 24 aa, Mus musculus
DGNVSRQCMLDFSSINIYVSSNDV

SEQ ID NO: 40 - protein, 15 aa, Mus musculus
SFMNVSLLLKIACNF

SEQ ID NO: 41 - protein, 4 aa, Mus musculus
HSNY

SEQ ID NO: 42 - protein, 4 aa, Mus musculus
ILTE

SEQ ID NO: 43 - protein, 8 aa, Mus musculus
RILHCKTF

SEQ ID NO: 44 - protein, 15 aa, Mus musculus
DELGYSFPNKVHQCH

SEQ ID NO: 45 - protein, 15 aa, Mus musculus
QVIKTKKKKNKHQPG

SEQ ID NO: 46 - protein, 16 aa, Homo sapiens
SWRMRLKLTGLKWLRC

SEQ ID NO: 47 - protein, 9 aa, Homo sapiens
NPCSDEEWC

SEQ ID NO: 48 - protein, 6 aa, Homo sapiens
YIKIMI

SEQ ID NO: 49 - protein, 8 aa, Homo sapiens
ECLKVMHV

SEQ ID NO: 50 - protein, 13 aa, Homo sapiens
YKCILVQMMYKVL

SEQ ID NO: 51 - protein, 8 aa, Homo sapiens
VSAFENCL

SEQ ID NO: 52 - protein, 10 aa, Homo sapiens
FLAFKLLNTP

SEQ ID NO: 53 - protein, 23 aa, Homo sapiens
FCIAKCFRMNFVIVLTPIKFISL